SEQUENCE LISTING (164 sequences)

SEQ ID NO: 1 - DNA, 22 bp, Homo sapiens
tgtctgcctattcaccgatttt

SEQ ID NO: 2 - DNA, 22 bp, Homo sapiens
ggcatgcagatcccacaggcgc

SEQ ID NO: 3 - DNA, 22 bp, Homo sapiens
ctgtaagatattcccatcccca

SEQ ID NO: 4 - DNA, 14 bp, Homo sapiens
tggtggggctgctc

SEQ ID NO: 5 - DNA, 2637 bp, Artificial Sequence
  Made in Lab - low activity TCRalpha megaTAL DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtgtcttctga

SEQ ID NO: 6 - RNA, 2637 nt, Artificial Sequence
  Made in Lab - low activity TCRalpha megaTAL RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugucuucuga

SEQ ID NO: 7 - protein, 878 aa, Artificial Sequence
  Made in Lab - low activity TCRalpha megaTAL protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRVF

SEQ ID NO: 8 - DNA, 2730 bp, Artificial Sequence
  Made in Lab - low activity TCRalpha megaTAL DNA construct
atgggatccgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaaccgtacaacgtctcctcccagtactttgtcaagaccacgggttgactccggatcaagtcgtcgcgatcgcgagccatgatggggggaagcaggcgctggaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgatggccatttcggcgtgaatctaaaaaaggtaaagggcaccgcaaaggtatacgtgggactgagattcgctatctcacagcacatcagagacaagaacctgatgaattcattgataacatacctaggctgtggttccatcagagagaagaacaagtctgagttcagatggctcgagttcgaagtaaccaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgttga

SEQ ID NO: 9 - RNA, 2730 nt, Artificial Sequence
  Made in Lab - low activity TCRalpha megaTAL RNA construct
augggauccgcgccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaaccguacaacgucuccucccaguacuuugucaagaccacggguugacuccggaucaagucgucgcgaucgcgagccaugauggggggaagcaggcgcuggaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucgcgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugauggccauuucggcgugaaucuaaaaaagguaaagggcaccgcaaagguauacgugggacugagauucgcuaucucacagcacaucagagacaagaaccugaugaauucauugauaacauaccuaggcugugguuccaucagagagaagaacaagucugaguucagauggcucgaguucgaaguaaccaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucguuga

SEQ ID NO: 10 - protein, 909 aa, Artificial Sequence
  Made in Lab - low activity TCRalpha megaTAL protein construct
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGDGHFGVNLKKVKGTAKVYVGLRFAISQHIRDKNLMNSLITYLGCGSIREKNKSEFRWLEFEVTKFSDINDKIIPVFQENTLIGVKLEDFEDWCKVAKLIEEKKHLTESGLDEIKKIKLNMNKGR

SEQ ID NO: 11 - DNA, 3372 bp, Artificial Sequence
  Made in Lab - low activity TCRalpha megaTAL-Trex2 DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagctccgaagcaccgagagccgaaaccttcgtgttcctcgacctggaagccactggcctgcccagtgtggagcccgagattgccgagctgtccctgttcgccgttcatcggtcctccctggaaaaccctgagcacgacgagagcggagcactggtgctgccaagggtgctggacaagctgaccctgtgcatgtgtcccgagcgtcccttcactgccaaggcatccgaaatcactggtctgagttcggaagggctggctcgttgtagaaaggctggattcgatggtgccgtggttcggactttgcaggcattcctgtccaggcaggcaggtccaatctgcttggtcgcccacaacggttttgactacgacttcccactgctctgtgccgaactgcgtcgcctgggagcacgccttcctcgggataccgtgtgcctggacaccctacccgctctgcgtggactggacagagcccactcacacggcactagggcgagaggacgccagggttacagcctcggttcactgttccatcggtatttcagggcagagccttccgcagcccattcagccgaaggcgatgtccacaccttgcttctgatcttcctgcatcgtgcagcagaactgctcgcctgggcagatgagcaggctcgtggatgggctcacattgagccgatgtacttgccacctgatgaccctagcctggaagcctga

SEQ ID NO: 12 - RNA, 3372 nt, Artificial Sequence
  Made in Lab - low activity TCRalpha megaTAL-Trex2 RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcuccgaagcaccgagagccgaaaccuucguguuccucgaccuggaagccacuggccugcccaguguggagcccgagauugccgagcugucccuguucgccguucaucgguccucccuggaaaacccugagcacgacgagagcggagcacuggugcugccaagggugcuggacaagcugacccugugcaugugucccgagcgucccuucacugccaaggcauccgaaaucacuggucugaguucggaagggcuggcucguuguagaaaggcuggauucgauggugccgugguucggacuuugcaggcauuccuguccaggcaggcagguccaaucugcuuggucgcccacaacgguuuugacuacgacuucccacugcucugugccgaacugcgucgccugggagcacgccuuccucgggauaccgugugccuggacacccuacccgcucugcguggacuggacagagcccacucacacggcacuagggcgagaggacgccaggguuacagccucgguucacuguuccaucgguauuucagggcagagccuuccgcagcccauucagccgaaggcgauguccacaccuugcuucugaucuuccugcaucgugcagcagaacugcucgccugggcagaugagcaggcucguggaugggcucacauugagccgauguacuugccaccugaugacccuagccuggaagccuga

SEQ ID NO: 13 - protein, 1123 aa, Artificial Sequence
  Made in Lab - low activity TCRalpha megaTAL-Trex2 protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 14 - DNA, 747 bp, Homo sapiens
atgggatcctgcaggccacctaagaagaaacgcaaagtctccgaagcaccgagagccgaaaccttcgtgttcctcgacctggaagccactggcctgcccagtgtggagcccgagattgccgagctgtccctgttcgccgttcatcggtcctccctggaaaaccctgagcacgacgagagcggagcactggtgctgccaagggtgctggacaagctgaccctgtgcatgtgtcccgagcgtcccttcactgccaaggcatccgaaatcactggtctgagttcggaagggctggctcgttgtagaaaggctggattcgatggtgccgtggttcggactttgcaggcattcctgtccaggcaggcaggtccaatctgcttggtcgcccacaacggttttgactacgacttcccactgctctgtgccgaactgcgtcgcctgggagcacgccttcctcgggataccgtgtgcctggacaccctacccgctctgcgtggactggacagagcccactcacacggcactagggcgagaggacgccagggttacagcctcggttcactgttccatcggtatttcagggcagagccttccgcagcccattcagccgaaggcgatgtccacaccttgcttctgatcttcctgcatcgtgcagcagaactgctcgcctgggcagatgagcaggctcgtggatgggctcacattgagccgatgtacttgccacctgatgaccctagcctggaagcctga

SEQ ID NO: 15 - RNA, 747 nt, Homo sapiens
augggauccugcaggccaccuaagaagaaacgcaaagucuccgaagcaccgagagccgaaaccuucguguuccucgaccuggaagccacuggccugcccaguguggagcccgagauugccgagcugucccuguucgccguucaucgguccucccuggaaaacccugagcacgacgagagcggagcacuggugcugccaagggugcuggacaagcugacccugugcaugugucccgagcgucccuucacugccaaggcauccgaaaucacuggucugaguucggaagggcuggcucguuguagaaaggcuggauucgauggugccgugguucggacuuugcaggcauuccuguccaggcaggcagguccaaucugcuuggucgcccacaacgguuuugacuacgacuucccacugcucugugccgaacugcgucgccugggagcacgccuuccucgggauaccgugugccuggacacccuacccgcucugcguggacuggacagagcccacucacacggcacuagggcgagaggacgccaggguuacagccucgguucacuguuccaucgguauuucagggcagagccuuccgcagcccauucagccgaaggcgauguccacaccuugcuucugaucuuccugcaucgugcagcagaacugcucgccugggcagaugagcaggcucguggaugggcucacauugagccgauguacuugccaccugaugacccuagccuggaagccuga

SEQ ID NO: 16 - protein, 248 aa, Homo sapiens
MGSCRPPKKKRKVSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 17 - DNA, 3384 bp, Artificial Sequence
  Made in Lab - low activity TCRalpha megaTAL-Trex2platypus DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagctccgaatcacagaaatgcgaaaccttcgtgttcctcgacctggaagccactggcctgcccgatgcgtctccccagattgccgaaatctccctgttcgccgttcatcggttctccctggaacaccctgcgtgggacgatagcggagtaccgctgctgccaagggtgctggacaagctgaccctgtgcatgaatcccgagcaacccttcactcccaaggcagccaagatcactggtctgaataacagatccctgactgataatagaaaggcttgtttcgatgggaccgtggttaaggctttgcgggagttcctgtccaggcaggcaagtccaatctgcttggtcgcccacaacggttttgcctacgacttcccactgctcaggaccgaactgggtcgcctggatgcaaaccttcctgagaatacctattgcctggacaccctacccgctctgaaaggactggacaaagcccacgatcacggcactaggtcgaatcatggcaagtcttaccgcctcggtgatctctacaggcagtatttcggtgacgatcctaaagcagcccattcagccgaaggcgatgtctacaccttggttatggtcttcctgcatcgggcaccagaactgctccgttgggcaggtgcggaggctcgttcatgggatgagattaggccgatgtactccccaggtcctcgtagatcaggtggcagaaggcatgatgcctga

SEQ ID NO: 18 - RNA, 3384 nt, Artificial Sequence
  Made in Lab - low activity TCRalpha megaTAL-Trex2platypus RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcuccgaaucacagaaaugcgaaaccuucguguuccucgaccuggaagccacuggccugcccgaugcgucuccccagauugccgaaaucucccuguucgccguucaucgguucucccuggaacacccugcgugggacgauagcggaguaccgcugcugccaagggugcuggacaagcugacccugugcaugaaucccgagcaacccuucacucccaaggcagccaagaucacuggucugaauaacagaucccugacugauaauagaaaggcuuguuucgaugggaccgugguuaaggcuuugcgggaguuccuguccaggcaggcaaguccaaucugcuuggucgcccacaacgguuuugccuacgacuucccacugcucaggaccgaacugggucgccuggaugcaaaccuuccugagaauaccuauugccuggacacccuacccgcucugaaaggacuggacaaagcccacgaucacggcacuaggucgaaucauggcaagucuuaccgccucggugaucucuacaggcaguauuucggugacgauccuaaagcagcccauucagccgaaggcgaugucuacaccuugguuauggucuuccugcaucgggcaccagaacugcuccguugggcaggugcggaggcucguucaugggaugagauuaggccgauguacuccccagguccucguagaucagguggcagaaggcaugaugccuga

SEQ ID NO: 19 - protein, 1127 aa, Artificial Sequence
  Made in Lab - low activity TCRalpha megaTAL-Trex2platypus protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASSESQKCETFVFLDLEATGLPDASPQIAEISLFAVHRFSLEHPAWDDSGVPLLPRVLDKLTLCMNPEQPFTPKAAKITGLNNRSLTDNRKACFDGTVVKALREFLSRQASPICLVAHNGFAYDFPLLRTELGRLDANLPENTYCLDTLPALKGLDKAHDHGTRSNHGKSYRLGDLYRQYFGDDPKAAHSAEGDVYTLVMVFLHRAPELLRWAGAEARSWDEIRPMYSPGPRRSGGRRHDA

SEQ ID NO: 20 - DNA, 3387 bp, Artificial Sequence
  Made in Lab - low activity TCRalpha megaTAL-Trex2opossum DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagcgccgaattaccgacatgcgaaaccttcgtgttcctcgacctggaagccactggcctgcccaatgctcatcccgagattgccgaaatctccctgttcgccattcatcggttctccctggaacaccctgagagagacgagagcggaacacttcaactgccaagggtgctggacaagctgaccctgtgcatgtgtcccgagcagaacttcactcccaaggcatccgaaatcactggtctgagtaatcagaacctggctgataatcacaaggctggattcaatggtgccgtgattcgggctttgcgggagttcctgaagaggcagaaaagtccaatctgcttggtcgcccacaacggttttgactacgacttcccactgctcaggaccgaacttcaacgcctgggagcagaccttcctggaggaaccgtgtgcctggacaccctacccgctctgcgtggactggacaaggtccaccatcacagcactagggcgtctcatggaaagaagtcttacagcctcggtaatctctacaggcggtatttcgatgacgagcctcaagcagcccattcagccgaaggcgatgtctacgccttggttatggtcttcctgcatcgggcacaagaactgctccaatgcgtacagacggaggctcagaattgggatgagattcagccgatgtataccccacgtaagcgtagactgtccggcggtagcctggatgcctga

SEQ ID NO: 21 - RNA, 3387 nt, Artificial Sequence
  Made in Lab - low activity TCRalpha megaTAL-Trex2opossum RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcgccgaauuaccgacaugcgaaaccuucguguuccucgaccuggaagccacuggccugcccaaugcucaucccgagauugccgaaaucucccuguucgccauucaucgguucucccuggaacacccugagagagacgagagcggaacacuucaacugccaagggugcuggacaagcugacccugugcaugugucccgagcagaacuucacucccaaggcauccgaaaucacuggucugaguaaucagaaccuggcugauaaucacaaggcuggauucaauggugccgugauucgggcuuugcgggaguuccugaagaggcagaaaaguccaaucugcuuggucgcccacaacgguuuugacuacgacuucccacugcucaggaccgaacuucaacgccugggagcagaccuuccuggaggaaccgugugccuggacacccuacccgcucugcguggacuggacaagguccaccaucacagcacuagggcgucucauggaaagaagucuuacagccucgguaaucucuacaggcgguauuucgaugacgagccucaagcagcccauucagccgaaggcgaugucuacgccuugguuauggucuuccugcaucgggcacaagaacugcuccaaugcguacagacggaggcucagaauugggaugagauucagccgauguauaccccacguaagcguagacuguccggcgguagccuggaugccuga

SEQ ID NO: 22 - protein, 1128 aa, Artificial Sequence
  Made in Lab - low activity TCRalpha megaTAL-Trex2opossum protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASAELPTCETFVFLDLEATGLPNAHPEIAEISLFAIHRFSLEHPERDESGTLQLPRVLDKLTLCMCPEQNFTPKASEITGLSNQNLADNHKAGFNGAVIRALREFLKRQKSPICLVAHNGFDYDFPLLRTELQRLGADLPGGTVCLDTLPALRGLDKVHHHSTRASHGKKSYSLGNLYRRYFDDEPQAAHSAEGDVYALVMVFLHRAQELLQCVQTEAQNWDEIQPMYTPRKRRLSGGSLDA

SEQ ID NO: 23 - DNA, 3372 bp, Artificial Sequence
  Made in Lab - low activity TCRalpha megaTAL-Trex2sheep DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagctccaaaggaccgagagccaagaccttcgtgttcctcgacctggaagccactggcctgcccagtgttgatcccgagattgccgagctgtccctgttcgccgttcatcggtcctccctggaagaccctgagtgcgacgagagcggatcaccgctgccgccaagggtgatggacaagctgaccctgtgcatgtctcccaaataccccttcactgccaaggcatccgaaatcactggtctgagtagggaagggctgggtcgttgtggaaaggctggattcgatgggaccgtggttcggactttgcaggcattcctgtccaggcaggaaggtccagtctgcttggtcgcccacaacggttttaactacgacttcccactgctctgtaccgaactgcgtcgcctgggagcacaccttcctcaagatacaatttgcctggataccctagtcgctcttcaagatctggacagagcccacggacacggcactagggcgcaaggaggcaagtcttacagcctcggttcactgttctgttgctatttccaggcagagccttccgcagcccattcagccgaaggcgatgtccacaccttgcttatggtcttcctgcatcgtgcagcagaactgctcgcctgggcagatgagcaggctctttcatgggctcacgttgagccgatgtacgtgccacctgatgacccaaggctggaagactga

SEQ ID NO: 24 - RNA, 3372 nt, Artificial Sequence
  Made in Lab - low activity TCRalpha megaTAL-Trex2sheep RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcuccaaaggaccgagagccaagaccuucguguuccucgaccuggaagccacuggccugcccaguguugaucccgagauugccgagcugucccuguucgccguucaucgguccucccuggaagacccugagugcgacgagagcggaucaccgcugccgccaagggugauggacaagcugacccugugcaugucucccaaauaccccuucacugccaaggcauccgaaaucacuggucugaguagggaagggcugggucguuguggaaaggcuggauucgaugggaccgugguucggacuuugcaggcauuccuguccaggcaggaagguccagucugcuuggucgcccacaacgguuuuaacuacgacuucccacugcucuguaccgaacugcgucgccugggagcacaccuuccucaagauacaauuugccuggauacccuagucgcucuucaagaucuggacagagcccacggacacggcacuagggcgcaaggaggcaagucuuacagccucgguucacuguucuguugcuauuuccaggcagagccuuccgcagcccauucagccgaaggcgauguccacaccuugcuuauggucuuccugcaucgugcagcagaacugcucgccugggcagaugagcaggcucuuucaugggcucacguugagccgauguacgugccaccugaugacccaaggcuggaagacuga

SEQ ID NO: 25 - protein, 1123 aa, Artificial Sequence
  Made in Lab - low activity TCRalpha megaTAL-Trex2sheep protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASSKGPRAKTFVFLDLEATGLPSVDPEIAELSLFAVHRSSLEDPECDESGSPLPPRVMDKLTLCMSPKYPFTAKASEITGLSREGLGRCGKAGFDGTVVRTLQAFLSRQEGPVCLVAHNGFNYDFPLLCTELRRLGAHLPQDTICLDTLVALQDLDRAHGHGTRAQGGKSYSLGSLFCCYFQAEPSAAHSAEGDVHTLLMVFLHRAAELLAWADEQALSWAHVEPMYVPPDDPRLED

SEQ ID NO: 26 - DNA, 3372 bp, Artificial Sequence
  Made in Lab - low activity TCRalpha megaTAL-Trex2armadillo DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagctcccaagcaccgcaagccgaaaccttcgtgttcctcgacctggaagccactggcctgcccaatgttgatcccgagattgccgaaatctccctgttcgccgttcatcggtcctccctggaacaccctgagagagacgaggccggagtacctgtgctgccaagggtgctggacaagctgaccctgtgcatgtctcccactcgtcccttcactgccaaggcatccgaaatcactggtctgagttcggaagggctggctcgttgtggaaaggctggattcgatgatactgcggctcagactttgcgggcattcctgggcaggcaggcaggtccactctgcttggtcgcccacaacggttttgactacgacttcccactgctcaggaccgaacttcaacgcctgggagcacgccttccagcgcataccgtgtgcctggacaccctacccgctctgcgtggactggacagagcccactcacacgctactagggcgcaaggacgcaagggttacagcctcggttcactgttcaggcggtatttccaggcagagccttccgcagcccattcagccgaaggcgatgtccgcaccttgcttctggtcttcctgcatcgtgcagcagaactgctcgcctgggcagatgggcgggctcgttcatgggctcacattgcgccgatgtacgcgccaccggaggcccctagcccggaagcctga

SEQ ID NO: 27 - RNA, 3372 nt, Artificial Sequence
  Made in Lab - low activity TCRalpha megaTAL-Trex2armadillo RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcucccaagcaccgcaagccgaaaccuucguguuccucgaccuggaagccacuggccugcccaauguugaucccgagauugccgaaaucucccuguucgccguucaucgguccucccuggaacacccugagagagacgaggccggaguaccugugcugccaagggugcuggacaagcugacccugugcaugucucccacucgucccuucacugccaaggcauccgaaaucacuggucugaguucggaagggcuggcucguuguggaaaggcuggauucgaugauacugcggcucagacuuugcgggcauuccugggcaggcaggcagguccacucugcuuggucgcccacaacgguuuugacuacgacuucccacugcucaggaccgaacuucaacgccugggagcacgccuuccagcgcauaccgugugccuggacacccuacccgcucugcguggacuggacagagcccacucacacgcuacuagggcgcaaggacgcaaggguuacagccucgguucacuguucaggcgguauuuccaggcagagccuuccgcagcccauucagccgaaggcgauguccgcaccuugcuucuggucuuccugcaucgugcagcagaacugcucgccugggcagaugggcgggcucguucaugggcucacauugcgccgauguacgcgccaccggaggccccuagcccggaagccuga

SEQ ID NO: 28 - protein, 1123 aa, Artificial Sequence
  Made in Lab - low activity TCRalpha megaTAL-Trex2armadillo protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASSQAPQAETFVFLDLEATGLPNVDPEIAEISLFAVHRSSLEHPERDEAGVPVLPRVLDKLTLCMSPTRPFTAKASEITGLSSEGLARCGKAGFDDTAAQTLRAFLGRQAGPLCLVAHNGFDYDFPLLRTELQRLGARLPAHTVCLDTLPALRGLDRAHSHATRAQGRKGYSLGSLFRRYFQAEPSAAHSAEGDVRTLLLVFLHRAAELLAWADGRARSWAHIAPMYAPPEAPSPEA

SEQ ID NO: 29 - DNA, 3372 bp, Artificial Sequence
  Made in Lab - low activity TCRalpha megaTAL-Trex2leopard DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagctccgaagtaccgagagccgaaaccttcgtgttcctcgacctggaagccactggcctgcccagtattgatcccgaggttgccgaaatctccctgttcgccgttcatcggtcctccctggaaaaccctgacagagacgagagcggagtacctgtgctgccaagggtgctggacaagctgaccctgtgcatgtctcccgagcgtcccttcactgccaaggcatccgaaatcactggtctgagttcggattccctggctcgttgtagaaaggctggattcgattcttccgtggttcggactttgcaggcattcctgtccaggcaggaaggtccagtctgcttggtcgcccacaacggttttgactacgacttcccactgctctgtaccgaactgcgtcgcctgggagcacaccttccaccggatacaatttgcctggacaccctacccgctctgcgtggactggacagagcccactcacacggcactagggcgcaaggattcaagggttacagcctcggttcactgttcaggcggtatttccatgcagagccttccgcagcccattcagccgaaggcgatgtccacaccttgcttatggtcttcctgcatcgtgcagcagaactgctctcctgggcagatgagcaggctcgttcatgggctcgcattaagccgatgtacgagccaccggatggccctagcgtggaagcctga

SEQ ID NO: 30 - RNA, 3372 nt, Artificial Sequence
  Made in Lab - low activity TCRalpha megaTAL-Trex2leopard RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcuccgaaguaccgagagccgaaaccuucguguuccucgaccuggaagccacuggccugcccaguauugaucccgagguugccgaaaucucccuguucgccguucaucgguccucccuggaaaacccugacagagacgagagcggaguaccugugcugccaagggugcuggacaagcugacccugugcaugucucccgagcgucccuucacugccaaggcauccgaaaucacuggucugaguucggauucccuggcucguuguagaaaggcuggauucgauucuuccgugguucggacuuugcaggcauuccuguccaggcaggaagguccagucugcuuggucgcccacaacgguuuugacuacgacuucccacugcucuguaccgaacugcgucgccugggagcacaccuuccaccggauacaauuugccuggacacccuacccgcucugcguggacuggacagagcccacucacacggcacuagggcgcaaggauucaaggguuacagccucgguucacuguucaggcgguauuuccaugcagagccuuccgcagcccauucagccgaaggcgauguccacaccuugcuuauggucuuccugcaucgugcagcagaacugcucuccugggcagaugagcaggcucguucaugggcucgcauuaagccgauguacgagccaccggauggcccuagcguggaagccuga

SEQ ID NO: 31 - protein, 1123 aa, Artificial Sequence
  Made in Lab - low activity TCRalpha megaTAL-Trex2leopard protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASSEVPRAETFVFLDLEATGLPSIDPEVAEISLFAVHRSSLENPDRDESGVPVLPRVLDKLTLCMSPERPFTAKASEITGLSSDSLARCRKAGFDSSVVRTLQAFLSRQEGPVCLVAHNGFDYDFPLLCTELRRLGAHLPPDTICLDTLPALRGLDRAHSHGTRAQGFKGYSLGSLFRRYFHAEPSAAHSAEGDVHTLLMVFLHRAAELLSWADEQARSWARIKPMYEPPDGPSVEA

SEQ ID NO: 32 - DNA, 3372 bp, Artificial Sequence
  Made in Lab - low activity TCRalpha megaTAL-Trex2mouse DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagctccgaaccgccgagagccgaaaccttcgtgttcctcgacctggaggccactggcctgccgaacatggacccggaaattgccgaaatctccctgttcgccgtgcaccggtcctccctggaaaaccctgagcgggacgatagcggttccctggtgctgcccagggtgctggacaagctgaccctgtgcatgtgccccgagcgccccttcactgccaaggcatccgaaatcactggtctgagctcggaatccctgatgcactgcggcaaggccggattcaacggcgccgtggtccggactttgcagggattcctgtccagacaggagggccccatctgcttggtcgcccacaacggttttgactacgacttccccctgctctgcaccgaactgcagcgcctgggagcccacctccctcaggacaccgtgtgcctggacaccctgcccgcactgcgcggactggacagagcccactcacacggcaccagggcgcagggacgcaagtcctacagcctcgcatcactgttccatcggtatttccaggccgagccttccgccgcccattccgccgaaggggatgtgcacaccttgcttctgatcttcctgcatcgcgctcccgaactgctggcctgggcggacgagcaggcccggtcctgggcccacattgagccgatgtacgtgccgcccgatggacctagcctggaagcctga

SEQ ID NO: 33 - RNA, 3372 nt, Artificial Sequence
  Made in Lab - low activity TCRalpha megaTAL-Trex2mouse RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcuccgaaccgccgagagccgaaaccuucguguuccucgaccuggaggccacuggccugccgaacauggacccggaaauugccgaaaucucccuguucgccgugcaccgguccucccuggaaaacccugagcgggacgauagcgguucccuggugcugcccagggugcuggacaagcugacccugugcaugugccccgagcgccccuucacugccaaggcauccgaaaucacuggucugagcucggaaucccugaugcacugcggcaaggccggauucaacggcgccgugguccggacuuugcagggauuccuguccagacaggagggccccaucugcuuggucgcccacaacgguuuugacuacgacuucccccugcucugcaccgaacugcagcgccugggagcccaccucccucaggacaccgugugccuggacacccugcccgcacugcgcggacuggacagagcccacucacacggcaccagggcgcagggacgcaaguccuacagccucgcaucacuguuccaucgguauuuccaggccgagccuuccgccgcccauuccgccgaaggggaugugcacaccuugcuucugaucuuccugcaucgcgcucccgaacugcuggccugggcggacgagcaggcccgguccugggcccacauugagccgauguacgugccgcccgauggaccuagccuggaagccuga

SEQ ID NO: 34 - protein, 1123 aa, Artificial Sequence
  Made in Lab - low activity TCRalpha megaTAL-Trex2mouse protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 35 - DNA, 3504 bp, Artificial Sequence
  Made in Lab - low activity TCRalpha megaTAL-hsRad1 DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcgccgcttctgacccaacagatccaagacgaggacgatcagtacagccttgttgccagccttgacaacgtgaggaatctctccactatcttgaaagctattcatttccgagaacatgccacctgtttcgcaactaagaacggtatcaaagtaacagtggagaatgcaaagtgtgtgcaagcaaatgccttcattcaggctggcatcttccaagagttcaaagttcaggaagagtctgttactttccgaatcaacctaactgtcctcttagactgtttatctatcttcggatcaagtccaatgccagggactttaactgcactccgaatgtgttaccaaggttacggataccctttgatgctgttcctcgaagaaggaggagtggtgacagtctgcaagatcaatacacaggaaccagaggagaccctggacttcgatttctgctccaccaatgttatcaacaagatcattctccagtcagaagggctccgtgaagcattctctgaattggatatgacgagtgaagtcctacagatcaccatgtctcctgacaagccttacttcaggttatctactttcggaaatgcaggaagttcccaccttgactatcccaaagattctgatttgatggaggcatttcattgtaatcagactcaagtgaacagatacaagatatccctactgaaaccctctacaaaggcattagtcctatcttgtaaggtatctattcggacagataacagaggcttcctttcattacagtatatgattagaaatgaagatggacaaatctgctttgtggagtattactgctgccctgatgaggaagttcctgaatctgagtcttga

SEQ ID NO: 36 - RNA, 3504 nt, Artificial Sequence
  Made in Lab - low activity TCRalpha megaTAL-hsRad1 RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucgccgcuucugacccaacagauccaagacgaggacgaucaguacagccuuguugccagccuugacaacgugaggaaucucuccacuaucuugaaagcuauucauuuccgagaacaugccaccuguuucgcaacuaagaacgguaucaaaguaacaguggagaaugcaaagugugugcaagcaaaugccuucauucaggcuggcaucuuccaagaguucaaaguucaggaagagucuguuacuuuccgaaucaaccuaacuguccucuuagacuguuuaucuaucuucggaucaaguccaaugccagggacuuuaacugcacuccgaauguguuaccaagguuacggauacccuuugaugcuguuccucgaagaaggaggaguggugacagucugcaagaucaauacacaggaaccagaggagacccuggacuucgauuucugcuccaccaauguuaucaacaagaucauucuccagucagaagggcuccgugaagcauucucugaauuggauaugacgagugaaguccuacagaucaccaugucuccugacaagccuuacuucagguuaucuacuuucggaaaugcaggaaguucccaccuugacuaucccaaagauucugauuugauggaggcauuucauuguaaucagacucaagugaacagauacaagauaucccuacugaaacccucuacaaaggcauuaguccuaucuuguaagguaucuauucggacagauaacagaggcuuccuuucauuacaguauaugauuagaaaugaagauggacaaaucugcuuuguggaguauuacugcugcccugaugaggaaguuccugaaucugagucuuga

SEQ ID NO: 37 - protein, 1167 aa, Artificial Sequence
  Made in Lab - low activity TCRalpha megaTAL-hsRad1 protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSPLLTQQIQDEDDQYSLVASLDNVRNLSTILKAIHFREHATCFATKNGIKVTVENAKCVQANAFIQAGIFQEFKVQEESVTFRINLTVLLDCLSIFGSSPMPGTLTALRMCYQGYGYPLMLFLEEGGVVTVCKINTQEPEETLDFDFCSTNVINKIILQSEGLREAFSELDMTSEVLQITMSPDKPYFRLSTFGNAGSSHLDYPKDSDLMEAFHCNQTQVNRYKISLLKPSTKALVLSCKVSIRTDNRGFLSLQYMIRNEDGQICFVEYYCCPDEEVPESES

SEQ ID NO: 38 - DNA, 3870 bp, Artificial Sequence
  Made in Lab - low activity TCRalpha megaTAL-hsRad9a DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagcggcaaaggagggttcttcagcaggtggtggcggagcgggaggacgatagggcaagtgtgtgagcagaagcagccagagggctgggtctgtggctgccctctgaccacgtccctctccctgctcttcgtggcccaactctccctccgaacggtgaactcctcccgctctgcctatgcctgctttctctttgctccgctcttcttccagcaataccaggcagccactcctggtcaagacctgctgcgatgtaagattctgatgaagtctttcctgtctgtcttccgctcactggcgatgctggagaagacggtggagaagtgctgcatctccctgaatggtcggagcagtcgcctggtagtccaactgcattgcaagttcggcgtgcggaagactcacaacctgtccttccaggactgtgagtcccttcaggccgtctttgacccagcctcgtgccctcacatgctccgagcaccagcacgggttctgggagaggctgttctgcccttctctcctgcactggctgaagtgacgctgggcattggccgtggacgcagggtcatcctgcgtagttaccacgaggaggaggcagacagcactgccaaagcgatggtgactgagatgtgccttggagaggaggatttccagcaactacaggcccaggaaggagttgccatcactttctgcctcaaggagttccgagggctcctgagtttcgcagagtcagcaaacttgaatcttagcattcatttcgatgctccaggcaggccagccatcttcaccatcaaggactctttgctggacggccactttgtcttagccacactctcagacaccgactcgcactcccaagacctgggctcaccagagcgtcaccagccagtgcctcaactccaggctcacagcacacctcaccctgacgactttgccaatgacgacattgactcttacatgatagcgatggaaaccactataggcaatgagggctcgcgtgtgctgccctccatttccctttcacctggccctcagcctcccaagagcccaggacctcactccgaggaggaagatgaggctgagcccagcacagtgcctgggactcctccacccaagaagttccgctcactgttcttcggctccatcctggctcctgttcgctcaccacagggtcctagccctgtgctggcggaagacagtgagggtgaaggctga

SEQ ID NO: 39 - RNA, 3870 nt, Artificial Sequence
  Made in Lab - low activity TCRalpha megaTAL-hsRad9a RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcggcaaaggaggguucuucagcaggugguggcggagcgggaggacgauagggcaagugugugagcagaagcagccagagggcugggucuguggcugcccucugaccacgucccucucccugcucuucguggcccaacucucccuccgaacggugaacuccucccgcucugccuaugccugcuuucucuuugcuccgcucuucuuccagcaauaccaggcagccacuccuggucaagaccugcugcgauguaagauucugaugaagucuuuccugucugucuuccgcucacuggcgaugcuggagaagacgguggagaagugcugcaucucccugaauggucggagcagucgccugguaguccaacugcauugcaaguucggcgugcggaagacucacaaccuguccuuccaggacugugagucccuucaggccgucuuugacccagccucgugcccucacaugcuccgagcaccagcacggguucugggagaggcuguucugcccuucucuccugcacuggcugaagugacgcugggcauuggccguggacgcagggucauccugcguaguuaccacgaggaggaggcagacagcacugccaaagcgauggugacugagaugugccuuggagaggaggauuuccagcaacuacaggcccaggaaggaguugccaucacuuucugccucaaggaguuccgagggcuccugaguuucgcagagucagcaaacuugaaucuuagcauucauuucgaugcuccaggcaggccagccaucuucaccaucaaggacucuuugcuggacggccacuuugucuuagccacacucucagacaccgacucgcacucccaagaccugggcucaccagagcgucaccagccagugccucaacuccaggcucacagcacaccucacccugacgacuuugccaaugacgacauugacucuuacaugauagcgauggaaaccacuauaggcaaugagggcucgcgugugcugcccuccauuucccuuucaccuggcccucagccucccaagagcccaggaccucacuccgaggaggaagaugaggcugagcccagcacagugccugggacuccuccacccaagaaguuccgcucacuguucuucggcuccauccuggcuccuguucgcucaccacaggguccuagcccugugcuggcggaagacagugagggugaaggcuga

SEQ ID NO: 40 - protein, 1289 aa, Artificial Sequence
  Made in Lab - low activity TCRalpha megaTAL-hsRad9a protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASGKGGFFSRWWRSGRTIGQVCEQKQPEGWVCGCPLTTSLSLLFVAQLSLRTVNSSRSAYACFLFAPLFFQQYQAATPGQDLLRCKILMKSFLSVFRSLAMLEKTVEKCCISLNGRSSRLVVQLHCKFGVRKTHNLSFQDCESLQAVFDPASCPHMLRAPARVLGEAVLPFSPALAEVTLGIGRGRRVILRSYHEEEADSTAKAMVTEMCLGEEDFQQLQAQEGVAITFCLKEFRGLLSFAESANLNLSIHFDAPGRPAIFTIKDSLLDGHFVLATLSDTDSHSQDLGSPERHQPVPQLQAHSTPHPDDFANDDIDSYMIAMETTIGNEGSRVLPSISLSPGPQPPKSPGPHSEEEDEAEPSTVPGTPPPKKFRSLFFGSILAPVRSPQGPSPVLAEDSEGEG

SEQ ID NO: 41 - DNA, 4086 bp, Artificial Sequence
  Made in Lab - low activity TCRalpha megaTAL-ExoI DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagcaacgatggcaagcagcaaagtacattccttttccacgactacgaaacctttggaacccaccccgctctggatcggccagcacagttcgctgccataagaaccgacaacgagttcaatgtgatcggtgaacccgaggtgttttactgcaaacctgcggacgattatctgcctcaaccgggagccgttctgattacgggaattaccccccaagaagcgcgtgccaagggcgaaaatgaagctgcatttgccgctagaatccatagcctgtttactgttccgaagacgtgtatcttgggatacaacaatgtacgctttgatgacgaagtaactcgtaacgtgttctatcggaacttctatgacccttacgcctggagttggcagcacgacaattctcgatgggatcttctggatgtgatgagagcctgctacgctctcaggccagagggcattaactggcctgagaacgacgatgggcttccgtcatttcgccttgagcatcttaccaaagcgaatggcatcgaacactccaatgcacacgacgctatggctgatgtctacgcgactattgcaatggcgaaattggtcaagacaagacagccgaggttgtttgactacctgtttacgcatcgaaacaaacataagctcatggcactgatagatgtccctcaaatgaagcccctcgttcacgtcagcggtatgttcggagcgtggagaggaaacacgtcctgggttgctcctctggcatggcatccagagaacagaaacgcggtaatcatggtcgatctcgcaggagacattagcccacttctcgaattggattccgacaccttgcgagaacgcctttacacagcgaaaactgatttgggtgataacgctgccgtacctgtcaaattggtacatatcaacaaatgtccggtgctcgcgcaagccaacaccctgagacctgaagacgcggatagactggggataaaccggcagcactgtttggataatctcaagatactcagggagaatccgcaggtgcgcgaaaaagtcgtagccatctttgcggaggcagaaccatttacgccgtctgacaatgtagatgctcagttatacaatggcttctttagtgacgctgaccgagctgcgatgaagattgtgctagagactgaaccgagaaacctctctgcattggacatcacttttgtcgataagcgaatagagaagttgctgttcaactatcgtgcccgaaactttccagggacactcgattacgcagagcaacaaaggtggcttgagcatcgcaggcaggtgtttaccccagagttcttgcagggttacgctgaggagattcaaatgttggcacagcagtatgcagatgataaggagaaagtcgcgttgctgaaggctctgtggcaatacgccgaagagatagtgtga

SEQ ID NO: 42 - RNA, 4086 nt, Artificial Sequence
  Made in Lab - low activity TCRalpha megaTAL-ExoI RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcaacgauggcaagcagcaaaguacauuccuuuuccacgacuacgaaaccuuuggaacccaccccgcucuggaucggccagcacaguucgcugccauaagaaccgacaacgaguucaaugugaucggugaacccgagguguuuuacugcaaaccugcggacgauuaucugccucaaccgggagccguucugauuacgggaauuaccccccaagaagcgcgugccaagggcgaaaaugaagcugcauuugccgcuagaauccauagccuguuuacuguuccgaagacguguaucuugggauacaacaauguacgcuuugaugacgaaguaacucguaacguguucuaucggaacuucuaugacccuuacgccuggaguuggcagcacgacaauucucgaugggaucuucuggaugugaugagagccugcuacgcucucaggccagagggcauuaacuggccugagaacgacgaugggcuuccgucauuucgccuugagcaucuuaccaaagcgaauggcaucgaacacuccaaugcacacgacgcuauggcugaugucuacgcgacuauugcaauggcgaaauuggucaagacaagacagccgagguuguuugacuaccuguuuacgcaucgaaacaaacauaagcucauggcacugauagaugucccucaaaugaagccccucguucacgucagcgguauguucggagcguggagaggaaacacguccuggguugcuccucuggcauggcauccagagaacagaaacgcgguaaucauggucgaucucgcaggagacauuagcccacuucucgaauuggauuccgacaccuugcgagaacgccuuuacacagcgaaaacugauuugggugauaacgcugccguaccugucaaauugguacauaucaacaaauguccggugcucgcgcaagccaacacccugagaccugaagacgcggauagacuggggauaaaccggcagcacuguuuggauaaucucaagauacucagggagaauccgcaggugcgcgaaaaagucguagccaucuuugcggaggcagaaccauuuacgccgucugacaauguagaugcucaguuauacaauggcuucuuuagugacgcugaccgagcugcgaugaagauugugcuagagacugaaccgagaaaccucucugcauuggacaucacuuuugucgauaagcgaauagagaaguugcuguucaacuaucgugcccgaaacuuuccagggacacucgauuacgcagagcaacaaagguggcuugagcaucgcaggcagguguuuaccccagaguucuugcaggguuacgcugaggagauucaaauguuggcacagcaguaugcagaugauaaggagaaagucgcguugcugaaggcucuguggcaauacgccgaagagauaguguga

SEQ ID NO: 43 - protein, 1361 aa, Artificial Sequence
  Made in Lab - low activity TCRalpha megaTAL-ExoI protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASNDGKQQSTFLFHDYETFGTHPALDRPAQFAAIRTDNEFNVIGEPEVFYCKPADDYLPQPGAVLITGITPQEARAKGENEAAFAARIHSLFTVPKTCILGYNNVRFDDEVTRNVFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLSALDITFVDKRIEKLLFNYRARNFPGTLDYAEQQRWLEHRRQVFTPEFLQGYAEEIQMLAQQYADDKEKVALLKALWQYAEEIV

SEQ ID NO: 44 - DNA, 3324 bp, Artificial Sequence
  Made in Lab - low activity TCRalpha megaTAL-ExoX DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagcctgcggatcatagacacggaaacctgtggactccagggtggtatcgtggagatagccagtgtcgatgtaattgatgggaaaatcgtaaacccgatgagccacctcgtccgaccggataggccgataagtccgcaggctatggctatacatagaatcacggaggctatggtagctgacaagccttggatcgaggacgttatcccacactactatgggagcgaatggtatgtagcgcacaacgcatcatttgataggcgcgtgcttcccgaaatgcctggggagtggatttgcacaatgaaactcgctcgccgactgtggcctggtatcaagtatagtaacatggcactttacaagacccgcaaactcaacgtacagacacctccgggtctccaccaccatagggcactctatgattgttacataaccgctgctctccttatagacataatgaatacctccgggtggaccgccgaacaaatggcagacataacgggaagacctagtttgatgacaacctttacatttggaaagtatcggggcaaggcggtgagcgatgtggccgaaagagatcccggttatcttagatggctcttcaataacttggattctatgtcacctgaacttcgccttaccctgaagcattaccttgagaatacctga

SEQ ID NO: 45 - RNA, 3324 nt, Artificial Sequence
  Made in Lab - low activity TCRalpha megaTAL-ExoX RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagccugcggaucauagacacggaaaccuguggacuccagggugguaucguggagauagccagugucgauguaauugaugggaaaaucguaaacccgaugagccaccucguccgaccggauaggccgauaaguccgcaggcuauggcuauacauagaaucacggaggcuaugguagcugacaagccuuggaucgaggacguuaucccacacuacuaugggagcgaaugguauguagcgcacaacgcaucauuugauaggcgcgugcuucccgaaaugccuggggaguggauuugcacaaugaaacucgcucgccgacuguggccugguaucaaguauaguaacauggcacuuuacaagacccgcaaacucaacguacagacaccuccgggucuccaccaccauagggcacucuaugauuguuacauaaccgcugcucuccuuauagacauaaugaauaccuccggguggaccgccgaacaaauggcagacauaacgggaagaccuaguuugaugacaaccuuuacauuuggaaaguaucggggcaaggcggugagcgauguggccgaaagagaucccgguuaucuuagauggcucuucaauaacuuggauucuaugucaccugaacuucgccuuacccugaagcauuaccuugagaauaccuga

SEQ ID NO: 46 - protein, 1107 aa, Artificial Sequence
  Made in Lab - low activity TCRalpha megaTAL-ExoX protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASLRIIDTETCGLQGGIVEIASVDVIDGKIVNPMSHLVRPDRPISPQAMAIHRITEAMVADKPWIEDVIPHYYGSEWYVAHNASFDRRVLPEMPGEWICTMKLARRLWPGIKYSNMALYKTRKLNVQTPPGLHHHRALYDCYITAALLIDIMNTSGWTAEQMADITGRPSLMTTFTFGKYRGKAVSDVAERDPGYLRWLFNNLDSMSPELRLTLKHYLENT

SEQ ID NO: 47 - DNA, 3537 bp, Artificial Sequence
  Made in Lab - low activity TCRalpha megaTAL-T5FEN DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagcagtaagagctgggggaagttcatagaggaggaggaagctgaaatggcaagcagacgcaatttgatgattgtggacggcactaatcttggtttcaggttcaaacataacaatagtaagaagccatttgccagcagctatgtttctacgatacaatcactggctaaaagttacagtgcgaggactacaatagtccttggcgacaaaggcaaatcagtgttcaggctcgaacatctccctgaatacaagggcaaccgcgatgagaagtatgcacagcgaacggaagaagagaaagccctggacgagcaattctttgaatatctcaaagatgcctttgaactgtgcaaaactacatttccaacctttaccatccggggagttgaggcagatgacatggcagcctacattgtaaaactgatagggcacctctacgaccatgtttggctcatatccactgacggagactgggacactttgttgaccgataaagtgtcaaggttctcattcactactcgtcgggaatatcatttgcgcgatatgtatgaacaccataacgtcgatgacgtggaacaattcatctctcttaaagcaatcatgggcgatctgggggacaacattaggggggttgaggggatcggagcgaaaaggggatacaatatcatacgggaatttggcaacgtccttgacatcatcgaccagctccccttgcccggaaagcagaagtacattcaaaacctcaacgcttcagaagaactgctctttaggaacttgatactggtagacctcccaacttactgcgtggatgcaatcgcggcggtaggacaggacgtgcttgacaaattcacgaaggacatactggagattgccgaacagtga

SEQ ID NO: 48 - RNA, 3537 nt, Artificial Sequence
  Made in Lab - low activity TCRalpha megaTAL-T5FEN RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcaguaagagcugggggaaguucauagaggaggaggaagcugaaauggcaagcagacgcaauuugaugauuguggacggcacuaaucuugguuucagguucaaacauaacaauaguaagaagccauuugccagcagcuauguuucuacgauacaaucacuggcuaaaaguuacagugcgaggacuacaauaguccuuggcgacaaaggcaaaucaguguucaggcucgaacaucucccugaauacaagggcaaccgcgaugagaaguaugcacagcgaacggaagaagagaaagcccuggacgagcaauucuuugaauaucucaaagaugccuuugaacugugcaaaacuacauuuccaaccuuuaccauccggggaguugaggcagaugacauggcagccuacauuguaaaacugauagggcaccucuacgaccauguuuggcucauauccacugacggagacugggacacuuuguugaccgauaaagugucaagguucucauucacuacucgucgggaauaucauuugcgcgauauguaugaacaccauaacgucgaugacguggaacaauucaucucucuuaaagcaaucaugggcgaucugggggacaacauuagggggguugaggggaucggagcgaaaaggggauacaauaucauacgggaauuuggcaacguccuugacaucaucgaccagcuccccuugcccggaaagcagaaguacauucaaaaccucaacgcuucagaagaacugcucuuuaggaacuugauacugguagaccucccaacuuacugcguggaugcaaucgcggcgguaggacaggacgugcuugacaaauucacgaaggacauacuggagauugccgaacaguga

SEQ ID NO: 49 - protein, 1178 aa, Artificial Sequence
  Made in Lab - low activity TCRalpha megaTAL-T5FEN protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 50 - DNA, 3342 bp, Artificial Sequence
  Made in Lab - low activity TCRalpha megaTAL-lambdaExo DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagcactcccgacatcatactgcaacgaaccggaattgacgttcgagcggtcgagcaaggggatgatgcctggcataagttgagacttggtgtgataactgcatcagaggtacataatgtcattgcgaagccccggagcgggaaaaagtggcccgacatgaagatgtcatactttcatacactgctggccgaggtctgcacgggtgttgctcccgaggttaatgccaaagcacttgcatggggtaaacagtacgaaaacgacgctcggaccctctttgagttcacatctggtgtgaatgtgacagagagcccaatcatttaccgagacgagagcatgagaacggcttgcagtcctgatggcctgtgcagcgacggtaacggactggagctaaagtgcccgtttactagccgagactttatgaagtttaggctcggtgggtttgaggccatcaagagtgcttacatggcgcaagtacagtattccatgtgggtcactcgcaaaaacgcttggtactttgctaactatgatccccgaatgaagagggagggcctgcattatgtggtcatagaacgggacgaaaagtatatggcgagcttcgacgaaattgtaccggagttcatagagaagatggatgaagcactcgccgagattgggttcgtctttggggaacaatggaggtga

SEQ ID NO: 51 - RNA, 3342 nt, Artificial Sequence
  Made in Lab - low activity TCRalpha megaTAL-lambdaExo RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcacucccgacaucauacugcaacgaaccggaauugacguucgagcggucgagcaaggggaugaugccuggcauaaguugagacuuggugugauaacugcaucagagguacauaaugucauugcgaagccccggagcgggaaaaaguggcccgacaugaagaugucauacuuucauacacugcuggccgaggucugcacggguguugcucccgagguuaaugccaaagcacuugcaugggguaaacaguacgaaaacgacgcucggacccucuuugaguucacaucuggugugaaugugacagagagcccaaucauuuaccgagacgagagcaugagaacggcuugcaguccugauggccugugcagcgacgguaacggacuggagcuaaagugcccguuuacuagccgagacuuuaugaaguuuaggcucgguggguuugaggccaucaagagugcuuacauggcgcaaguacaguauuccaugugggucacucgcaaaaacgcuugguacuuugcuaacuaugauccccgaaugaagagggagggccugcauuauguggucauagaacgggacgaaaaguauauggcgagcuucgacgaaauuguaccggaguucauagagaagauggaugaagcacucgccgagauuggguucgucuuuggggaacaauggagguga

SEQ ID NO: 52 - protein, 1113 aa, Artificial Sequence
  Made in Lab - low activity TCRalpha megaTAL-lambdaExo protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 53 - DNA, 4398 bp, Artificial Sequence
  Made in Lab - low activity TCRalpha megaTAL-RecJ DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgtggtggaggcggtagcggaggcggagggtcggctagcaagcaacaaatccatttgagacgaagggaggttgacgagactgctgatctcccggctgcggagttgcctccgctattgcgtcgcctgtacgcctctcggggtgtacgctccgctcaagaactggaacgatcagtaaagggtatgctgccctggcaacagctttccggggtggagaaggccgtggagatactatacaatgcgttccgagaagggacgagaatcatagtagttggggactttgatgcggatggcgcgacttccactgccctgtctgtgctggcgatgcggagtcttgggtgttccaacatagactacctcgttccaaatcggtttgaagatggatacggcctttcacccgaagttgtggaccaagcccacgcgaggggagcccaacttatagtgacagtggataacggaattagctcacatgcaggagtagagcacgcaaggtcactgggtatccccgtgattgtcaccgaccatcacctcccaggagacactttgcctgctgctgaggccatcattaaccctaatcttagggactgcaactttcctagtaagagtctggctggggttggagtcgcattctaccttatgctggccctccgcacttttctccgagatcaaggctggtttgatgaacgaaacattgctatcccaaatcttgcagaactccttgaccttgtggctttgggaacagtcgccgatgtcgtgccgctggacgctaacaatcggatattgacttggcaggggatgtcacgcatccgtgcgggcaagtgcaggccaggtatcaaggcattgttggaagtcgccaaccgggacgcacaaaaacttgccgcaagcgacctcggttttgcacttggtcctcgccttaacgcagcgggtagattggatgatatgtccgtgggcgtggcgctcttgttgtgcgacaacataggtgaagcgagagttcttgcaaatgagttggacgcgctcaatcagactaggaaagagatagaacaaggaatgcagatcgaggcactgaccctttgtgaaaagctggagcgatcacgggataccttgcctggaggtctcgcaatgtaccatcctgagtggcatcagggcgtcgtaggtatcttggcgtctaggatcaaagagaggttccatcgcccagtgattgcatttgcgccagcaggtgatggcactctgaaaggatctggccgctccatccaaggtctccacatgagagatgcacttgagaggttggacacactttacccaggcatgatgttgaaattcggtgggcacgctatggctgctggtttgagcttggaagaagataagttcaaactgttccaacagaggttcggcgaactggtaactgagtggcttgatccttcattgctccaaggtgaggtggtatcagatgggccactcagcccagccgaaatgactatggaagtagcccagctcctcagagatgcaggtccttggggtcaaatgttccccgagcccttgttcgacgggcatttcaggttgctccagcagaggttggtaggcgagcgccacctcaaagtgatggtcgaacctgtaggtggaggtccgcttctggacggaattgctttcaacgtagacacggcgctgtggcccgacaatggcgttagggaggtacaacttgcctacaaactcgacatcaacgagtttagaggtaacagaagcctccagatcatcattgacaatatctggcccatttga

SEQ ID NO: 54 - RNA, 4398 nt, Artificial Sequence
  Made in Lab - low activity TCRalpha megaTAL-RecJ RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugacuccggaccaagugguggcuaucgccagccacgauggcggcaagcaagcgcucgaaacggugcagcggcuguugccggugcugugccaggaccauggccugaccccggaccaagugguggcuaucgccagcaacauuggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacgguggcggcaagcaagcgcucgaaacggugcagcggcuguugccggugcugugccaggaccauggccugaccccggaccaagugguggcuaucgccagcaacaauggcggcaagcaagcgcucgaaacggugcagcggcuguugccggugcugugccaggaccauggccugaccccggaccaagugguggcuaucgccagcaacauuggcggcaagcaagcgcucgaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgugguggaggcgguagcggaggcggagggucggcuagcaagcaacaaauccauuugagacgaagggagguugacgagacugcugaucucccggcugcggaguugccuccgcuauugcgucgccuguacgccucucgggguguacgcuccgcucaagaacuggaacgaucaguaaaggguaugcugcccuggcaacagcuuuccgggguggagaaggccguggagauacuauacaaugcguuccgagaagggacgagaaucauaguaguuggggacuuugaugcggauggcgcgacuuccacugcccugucugugcuggcgaugcggagucuuggguguuccaacauagacuaccucguuccaaaucgguuugaagauggauacggccuuucacccgaaguuguggaccaagcccacgcgaggggagcccaacuuauagugacaguggauaacggaauuagcucacaugcaggaguagagcacgcaaggucacuggguauccccgugauugucaccgaccaucaccucccaggagacacuuugccugcugcugaggccaucauuaacccuaaucuuagggacugcaacuuuccuaguaagagucuggcugggguuggagucgcauucuaccuuaugcuggcccuccgcacuuuucuccgagaucaaggcugguuugaugaacgaaacauugcuaucccaaaucuugcagaacuccuugaccuuguggcuuugggaacagucgccgaugucgugccgcuggacgcuaacaaucggauauugacuuggcaggggaugucacgcauccgugcgggcaagugcaggccagguaucaaggcauuguuggaagucgccaaccgggacgcacaaaaacuugccgcaagcgaccucgguuuugcacuugguccucgccuuaacgcagcggguagauuggaugauauguccgugggcguggcgcucuuguugugcgacaacauaggugaagcgagaguucuugcaaaugaguuggacgcgcucaaucagacuaggaaagagauagaacaaggaaugcagaucgaggcacugacccuuugugaaaagcuggagcgaucacgggauaccuugccuggaggucucgcaauguaccauccugaguggcaucagggcgucguagguaucuuggcgucuaggaucaaagagagguuccaucgcccagugauugcauuugcgccagcaggugauggcacucugaaaggaucuggccgcuccauccaaggucuccacaugagagaugcacuugagagguuggacacacuuuacccaggcaugauguugaaauucggugggcacgcuauggcugcugguuugagcuuggaagaagauaaguucaaacuguuccaacagagguucggcgaacugguaacugaguggcuugauccuucauugcuccaaggugaggugguaucagaugggccacucagcccagccgaaaugacuauggaaguagcccagcuccucagagaugcagguccuuggggucaaauguuccccgagcccuuguucgacgggcauuucagguugcuccagcagagguugguaggcgagcgccaccucaaagugauggucgaaccuguagguggagguccgcuucuggacggaauugcuuucaacguagacacggcgcuguggcccgacaauggcguuagggagguacaacuugccuacaaacucgacaucaacgaguuuagagguaacagaagccuccagaucaucauugacaauaucuggcccauuuga

SEQ ID NO: 55 - protein, 1465 aa, Artificial Sequence
  Made in Lab - low activity TCRalpha megaTAL-RecJ protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRGGGGSGGGGSASKQQIHLRRREVDETADLPAAELPPLLRRLYASRGVRSAQELERSVKGMLPWQQLSGVEKAVEILYNAFREGTRIIVVGDFDADGATSTALSVLAMRSLGCSNIDYLVPNRFEDGYGLSPEVVDQAHARGAQLIVTVDNGISSHAGVEHARSLGIPVIVTDHHLPGDTLPAAEAIINPNLRDCNFPSKSLAGVGVAFYLMLALRTFLRDQGWFDERNIAIPNLAELLDLVALGTVADVVPLDANNRILTWQGMSRIRAGKCRPGIKALLEVANRDAQKLAASDLGFALGPRLNAAGRLDDMSVGVALLLCDNIGEARVLANELDALNQTRKEIEQGMQIEALTLCEKLERSRDTLPGGLAMYHPEWHQGVVGILASRIKERFHRPVIAFAPAGDGTLKGSGRSIQGLHMRDALERLDTLYPGMMLKFGGHAMAAGLSLEEDKFKLFQQRFGELVTEWLDPSLLQGEVVSDGPLSPAEMTMEVAQLLRDAGPWGQMFPEPLFDGHFRLLQQRLVGERHLKVMVEPVGGGPLLDGIAFNVDTALWPDNGVREVQLAYKLDINEFRGNRSLQIIIDNIWPI

SEQ ID NO: 56 - DNA, 2733 bp, Artificial Sequence
  Made in Lab - high activity Cb1-b megaTAL DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacttaacacccgatcaagtggtagcgatagcgtcaaacggcgggggtaaacaggctttggagacggtacagcggttattgccggtactctgccaggaccacggattgacaccggaccaagtggtggcgattgcgtccaacaacggaggcaagcaggcactagagactgtccaacggcttcttcccgttctttgtcaggatcatgggctaacccctgatcaggtagtcgctatagcttcaaatggagggggcaagcaagcactggagactgttcaacgactcctgccagtgctctgccaagaccacggacttacaccagatcaagtggttgctattgcctccaatggtggcgggaaacaagcgttggaaactgtgcagagactgttacctgtcttgtgtcaagaccacggcctcacgccagatcaggtggtagccatagcgtcgaacattggtggtaagcaagcccttgaaacggtccagcgtcttctgccggtgttgtgccaggaccacggactaacgccggatcaggtcgtagccattgcttcaaatgggggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaacattggaggtaagcaagcactggagactgtccagagattgttacccgtactatgtcaagatcatggtttgacgcctgatcaggttgttgcgatagccagcaataacggagggaaacaggctcttgaaaccgtacagcgacttctcccagtcttgtgccaagatcacgggcttactcctgatcaagtcgtagctatcgccagcaataacggtggaaaacaggccctggaaaccgtacaacgtctcctcccagtactttgtcaagaccacgggttgactccggatcaagtcgtcgcgatcgcgagcaatggtggggggaagcaggcgctggaaactgttcagagactgctgcctgtactttgtcaggaccatggtctgacacctgaccaagttgtggcgatagccagtaatatcgggggaaaacaggcactagagagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcgctgatgccgaaggatgcttccgactagacatccgcaacgcaaacgatttaagagccggatacagaactagactggccttcgaaatcgtactgcacaacaaggacaaatcgattctggagaatatccagtcgacttggaaggtcggcacaatctacaacgcgggcgacaacgcagtcagactgcaagtcacacgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgctcccttatcaataagaacattccgaatctcaaatggctggctggattcacatctggtgacggctcgttcgtggtggaactaaagaagagaagaagccccgtcaaggtaggagtgcggctgcgattcagcatcacccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgttga

SEQ ID NO: 57 - RNA, 2733 nt, Artificial Sequence
  Made in Lab - high activity Cb1-b megaTAL RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaacggcggggguaaacaggcuuuggagacgguacagcgguuauugccgguacucugccaggaccacggauugacaccggaccaagugguggcgauugcguccaacaacggaggcaagcaggcacuagagacuguccaacggcuucuucccguucuuugucaggaucaugggcuaaccccugaucagguagucgcuauagcuucaaauggagggggcaagcaagcacuggagacuguucaacgacuccugccagugcucugccaagaccacggacuuacaccagaucaagugguugcuauugccuccaaugguggcgggaaacaagcguuggaaacugugcagagacuguuaccugucuugugucaagaccacggccucacgccagaucaggugguagccauagcgucgaacauuggugguaagcaagcccuugaaacgguccagcgucuucugccgguguugugccaggaccacggacuaacgccggaucaggucguagccauugcuucaaaugggggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacauuggagguaagcaagcacuggagacuguccagagauuguuacccguacuaugucaagaucaugguuugacgccugaucagguuguugcgauagccagcaauaacggagggaaacaggcucuugaaaccguacagcgacuucucccagucuugugccaagaucacgggcuuacuccugaucaagucguagcuaucgccagcaauaacgguggaaaacaggcccuggaaaccguacaacgucuccucccaguacuuugucaagaccacggguugacuccggaucaagucgucgcgaucgcgagcaaugguggggggaagcaggcgcuggaaacuguucagagacugcugccuguacuuugucaggaccauggucugacaccugaccaaguuguggcgauagccaguaauaucgggggaaaacaggcacuagagagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucgcgcagagaguccaucaacccauggauucugacugguuucgcugaugccgaaggaugcuuccgacuagacauccgcaacgcaaacgauuuaagagccggauacagaacuagacuggccuucgaaaucguacugcacaacaaggacaaaucgauucuggagaauauccagucgacuuggaaggucggcacaaucuacaacgcgggcgacaacgcagucagacugcaagucacacguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcucccuuaucaauaagaacauuccgaaucucaaauggcuggcuggauucacaucuggugacggcucguucgugguggaacuaaagaagagaagaagccccgucaagguaggagugcggcugcgauucagcaucacccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucguuga

SEQ ID NO: 58 - protein, 910 aa, Artificial Sequence
  Made in Lab - high activity Cb1-b megaTAL protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPENISKERSLINKNIPNLKWLAGFTSGDGSFVVELKKRRSPVKVGVRLRFSITQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGR

SEQ ID NO: 59 - DNA, 3474 bp, Artificial Sequence
  Made in Lab - high activity Cb1-b megaTAL-Trex2 DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacttaacacccgatcaagtggtagcgatagcgtcaaacggcgggggtaaacaggctttggagacggtacagcggttattgccggtactctgccaggaccacggattgacaccggaccaagtggtggcgattgcgtccaacaacggaggcaagcaggcactagagactgtccaacggcttcttcccgttctttgtcaggatcatgggctaacccctgatcaggtagtcgctatagcttcaaatggagggggcaagcaagcactggagactgttcaacgactcctgccagtgctctgccaagaccacggacttacaccagatcaagtggttgctattgcctccaatggtggcgggaaacaagcgttggaaactgtgcagagactgttacctgtcttgtgtcaagaccacggcctcacgccagatcaggtggtagccatagcgtcgaacattggtggtaagcaagcccttgaaacggtccagcgtcttctgccggtgttgtgccaggaccacggactaacgccggatcaggtcgtagccattgcttcaaatgggggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaacattggaggtaagcaagcactggagactgtccagagattgttacccgtactatgtcaagatcatggtttgacgcctgatcaggttgttgcgatagccagcaataacggagggaaacaggctcttgaaaccgtacagcgacttctcccagtcttgtgccaagatcacgggcttactcctgatcaagtcgtagctatcgccagcaataacggtggaaaacaggccctggaaaccgtacaacgtctcctcccagtactttgtcaagaccacgggttgactccggatcaagtcgtcgcgatcgcgagcaatggtggggggaagcaggcgctggaaactgttcagagactgctgcctgtactttgtcaggaccatggtctgacacctgaccaagttgtggcgatagccagtaatatcgggggaaaacaggcactagagagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcgctgatgccgaaggatgcttccgactagacatccgcaacgcaaacgatttaagagccggatacagaactagactggccttcgaaatcgtactgcacaacaaggacaaatcgattctggagaatatccagtcgacttggaaggtcggcacaatctacaacgcgggcgacaacgcagtcagactgcaagtcacacgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgctcccttatcaataagaacattccgaatctcaaatggctggctggattcacatctggtgacggctcgttcgtggtggaactaaagaagagaagaagccccgtcaaggtaggagtgcggctgcgattcagcatcacccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtggtggaggcggtagcggaggcggagggtcggctagctccgaagcaccgagagccgaaaccttcgtgttcctcgacctggaagccactggcctgcccagtgtggagcccgagattgccgagctgtccctgttcgccgttcatcggtcctccctggaaaaccctgagcacgacgagagcggagcactggtgctgccaagggtgctggacaagctgaccctgtgcatgtgtcccgagcgtcccttcactgccaaggcatccgaaatcactggtctgagttcggaagggctggctcgttgtagaaaggctggattcgatggtgccgtggttcggactttgcaggcattcctgtccaggcaggcaggtccaatctgcttggtcgcccacaacggttttgactacgacttcccactgctctgtgccgaactgcgtcgcctgggagcacgccttcctcgggataccgtgtgcctggacaccctacccgctctgcgtggactggacagagcccactcacacggcactagggcgagaggacgccagggttacagcctcggttcactgttccatcggtatttcagggcagagccttccgcagcccattcagccgaaggcgatgtccacaccttgcttctgatcttcctgcatcgtgcagcagaactgctcgcctgggcagatgagcaggctcgtggatgggctcacattgagccgatgtacttgccacctgatgaccctagcctggaagcctga

SEQ ID NO: 60 - RNA, 3474 nt, Artificial Sequence
  Made in Lab - high activity Cb1-b megaTAL-Trex2 RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaacggcggggguaaacaggcuuuggagacgguacagcgguuauugccgguacucugccaggaccacggauugacaccggaccaagugguggcgauugcguccaacaacggaggcaagcaggcacuagagacuguccaacggcuucuucccguucuuugucaggaucaugggcuaaccccugaucagguagucgcuauagcuucaaauggagggggcaagcaagcacuggagacuguucaacgacuccugccagugcucugccaagaccacggacuuacaccagaucaagugguugcuauugccuccaaugguggcgggaaacaagcguuggaaacugugcagagacuguuaccugucuugugucaagaccacggccucacgccagaucaggugguagccauagcgucgaacauuggugguaagcaagcccuugaaacgguccagcgucuucugccgguguugugccaggaccacggacuaacgccggaucaggucguagccauugcuucaaaugggggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacauuggagguaagcaagcacuggagacuguccagagauuguuacccguacuaugucaagaucaugguuugacgccugaucagguuguugcgauagccagcaauaacggagggaaacaggcucuugaaaccguacagcgacuucucccagucuugugccaagaucacgggcuuacuccugaucaagucguagcuaucgccagcaauaacgguggaaaacaggcccuggaaaccguacaacgucuccucccaguacuuugucaagaccacggguugacuccggaucaagucgucgcgaucgcgagcaaugguggggggaagcaggcgcuggaaacuguucagagacugcugccuguacuuugucaggaccauggucugacaccugaccaaguuguggcgauagccaguaauaucgggggaaaacaggcacuagagagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucgcgcagagaguccaucaacccauggauucugacugguuucgcugaugccgaaggaugcuuccgacuagacauccgcaacgcaaacgauuuaagagccggauacagaacuagacuggccuucgaaaucguacugcacaacaaggacaaaucgauucuggagaauauccagucgacuuggaaggucggcacaaucuacaacgcgggcgacaacgcagucagacugcaagucacacguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcucccuuaucaauaagaacauuccgaaucucaaauggcuggcuggauucacaucuggugacggcucguucgugguggaacuaaagaagagaagaagccccgucaagguaggagugcggcugcgauucagcaucacccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgugguggaggcgguagcggaggcggagggucggcuagcuccgaagcaccgagagccgaaaccuucguguuccucgaccuggaagccacuggccugcccaguguggagcccgagauugccgagcugucccuguucgccguucaucgguccucccuggaaaacccugagcacgacgagagcggagcacuggugcugccaagggugcuggacaagcugacccugugcaugugucccgagcgucccuucacugccaaggcauccgaaaucacuggucugaguucggaagggcuggcucguuguagaaaggcuggauucgauggugccgugguucggacuuugcaggcauuccuguccaggcaggcagguccaaucugcuuggucgcccacaacgguuuugacuacgacuucccacugcucugugccgaacugcgucgccugggagcacgccuuccucgggauaccgugugccuggacacccuacccgcucugcguggacuggacagagcccacucacacggcacuagggcgagaggacgccaggguuacagccucgguucacuguuccaucgguauuucagggcagagccuuccgcagcccauucagccgaaggcgauguccacaccuugcuucugaucuuccugcaucgugcagcagaacugcucgccugggcagaugagcaggcucguggaugggcucacauugagccgauguacuugccaccugaugacccuagccuggaagccuga

SEQ ID NO: 61 - protein, 1157 aa, Artificial Sequence
  Made in Lab - high activity Cb1-b megaTAL-Trex2 protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPENISKERSLINKNIPNLKWLAGFTSGDGSFVVELKKRRSPVKVGVRLRFSITQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRGGGGSGGGGSASSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 62 - DNA, 3426 bp, Artificial Sequence
  Made in Lab - high activity Cb1-b megaTAL-ExoX DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacttaacacccgatcaagtggtagcgatagcgtcaaacggcgggggtaaacaggctttggagacggtacagcggttattgccggtactctgccaggaccacggattgacaccggaccaagtggtggcgattgcgtccaacaacggaggcaagcaggcactagagactgtccaacggcttcttcccgttctttgtcaggatcatgggctaacccctgatcaggtagtcgctatagcttcaaatggagggggcaagcaagcactggagactgttcaacgactcctgccagtgctctgccaagaccacggacttacaccagatcaagtggttgctattgcctccaatggtggcgggaaacaagcgttggaaactgtgcagagactgttacctgtcttgtgtcaagaccacggcctcacgccagatcaggtggtagccatagcgtcgaacattggtggtaagcaagcccttgaaacggtccagcgtcttctgccggtgttgtgccaggaccacggactaacgccggatcaggtcgtagccattgcttcaaatgggggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaacattggaggtaagcaagcactggagactgtccagagattgttacccgtactatgtcaagatcatggtttgacgcctgatcaggttgttgcgatagccagcaataacggagggaaacaggctcttgaaaccgtacagcgacttctcccagtcttgtgccaagatcacgggcttactcctgatcaagtcgtagctatcgccagcaataacggtggaaaacaggccctggaaaccgtacaacgtctcctcccagtactttgtcaagaccacgggttgactccggatcaagtcgtcgcgatcgcgagcaatggtggggggaagcaggcgctggaaactgttcagagactgctgcctgtactttgtcaggaccatggtctgacacctgaccaagttgtggcgatagccagtaatatcgggggaaaacaggcactagagagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcgctgatgccgaaggatgcttccgactagacatccgcaacgcaaacgatttaagagccggatacagaactagactggccttcgaaatcgtactgcacaacaaggacaaatcgattctggagaatatccagtcgacttggaaggtcggcacaatctacaacgcgggcgacaacgcagtcagactgcaagtcacacgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgctcccttatcaataagaacattccgaatctcaaatggctggctggattcacatctggtgacggctcgttcgtggtggaactaaagaagagaagaagccccgtcaaggtaggagtgcggctgcgattcagcatcacccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtggtggaggcggtagcggaggcggagggtcggctagcctgcggatcatagacacggaaacctgtggactccagggtggtatcgtggagatagccagtgtcgatgtaattgatgggaaaatcgtaaacccgatgagccacctcgtccgaccggataggccgataagtccgcaggctatggctatacatagaatcacggaggctatggtagctgacaagccttggatcgaggacgttatcccacactactatgggagcgaatggtatgtagcgcacaacgcatcatttgataggcgcgtgcttcccgaaatgcctggggagtggatttgcacaatgaaactcgctcgccgactgtggcctggtatcaagtatagtaacatggcactttacaagacccgcaaactcaacgtacagacacctccgggtctccaccaccatagggcactctatgattgttacataaccgctgctctccttatagacataatgaatacctccgggtggaccgccgaacaaatggcagacataacgggaagacctagtttgatgacaacctttacatttggaaagtatcggggcaaggcggtgagcgatgtggccgaaagagatcccggttatcttagatggctcttcaataacttggattctatgtcacctgaacttcgccttaccctgaagcattaccttgagaatacctga

SEQ ID NO: 63 - RNA, 3426 nt, Artificial Sequence
  Made in Lab - high activity Cb1-b megaTAL-ExoX RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaacggcggggguaaacaggcuuuggagacgguacagcgguuauugccgguacucugccaggaccacggauugacaccggaccaagugguggcgauugcguccaacaacggaggcaagcaggcacuagagacuguccaacggcuucuucccguucuuugucaggaucaugggcuaaccccugaucagguagucgcuauagcuucaaauggagggggcaagcaagcacuggagacuguucaacgacuccugccagugcucugccaagaccacggacuuacaccagaucaagugguugcuauugccuccaaugguggcgggaaacaagcguuggaaacugugcagagacuguuaccugucuugugucaagaccacggccucacgccagaucaggugguagccauagcgucgaacauuggugguaagcaagcccuugaaacgguccagcgucuucugccgguguugugccaggaccacggacuaacgccggaucaggucguagccauugcuucaaaugggggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacauuggagguaagcaagcacuggagacuguccagagauuguuacccguacuaugucaagaucaugguuugacgccugaucagguuguugcgauagccagcaauaacggagggaaacaggcucuugaaaccguacagcgacuucucccagucuugugccaagaucacgggcuuacuccugaucaagucguagcuaucgccagcaauaacgguggaaaacaggcccuggaaaccguacaacgucuccucccaguacuuugucaagaccacggguugacuccggaucaagucgucgcgaucgcgagcaaugguggggggaagcaggcgcuggaaacuguucagagacugcugccuguacuuugucaggaccauggucugacaccugaccaaguuguggcgauagccaguaauaucgggggaaaacaggcacuagagagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucgcgcagagaguccaucaacccauggauucugacugguuucgcugaugccgaaggaugcuuccgacuagacauccgcaacgcaaacgauuuaagagccggauacagaacuagacuggccuucgaaaucguacugcacaacaaggacaaaucgauucuggagaauauccagucgacuuggaaggucggcacaaucuacaacgcgggcgacaacgcagucagacugcaagucacacguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcucccuuaucaauaagaacauuccgaaucucaaauggcuggcuggauucacaucuggugacggcucguucgugguggaacuaaagaagagaagaagccccgucaagguaggagugcggcugcgauucagcaucacccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgugguggaggcgguagcggaggcggagggucggcuagccugcggaucauagacacggaaaccuguggacuccagggugguaucguggagauagccagugucgauguaauugaugggaaaaucguaaacccgaugagccaccucguccgaccggauaggccgauaaguccgcaggcuauggcuauacauagaaucacggaggcuaugguagcugacaagccuuggaucgaggacguuaucccacacuacuaugggagcgaaugguauguagcgcacaacgcaucauuugauaggcgcgugcuucccgaaaugccuggggaguggauuugcacaaugaaacucgcucgccgacuguggccugguaucaaguauaguaacauggcacuuuacaagacccgcaaacucaacguacagacaccuccgggucuccaccaccauagggcacucuaugauuguuacauaaccgcugcucuccuuauagacauaaugaauaccuccggguggaccgccgaacaaauggcagacauaacgggaagaccuaguuugaugacaaccuuuacauuuggaaaguaucggggcaaggcggugagcgauguggccgaaagagaucccgguuaucuuagauggcucuucaauaacuuggauucuaugucaccugaacuucgccuuacccugaagcauuaccuugagaauaccuga

SEQ ID NO: 64 - protein, 1141 aa, Artificial Sequence
  Made in Lab - high activity Cb1-b megaTAL-ExoX protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPENISKERSLINKNIPNLKWLAGFTSGDGSFVVELKKRRSPVKVGVRLRFSITQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRGGGGSGGGGSASLRIIDTETCGLQGGIVEIASVDVIDGKIVNPMSHLVRPDRPISPQAMAIHRITEAMVADKPWIEDVIPHYYGSEWYVAHNASFDRRVLPEMPGEWICTMKLARRLWPGIKYSNMALYKTRKLNVQTPPGLHHHRALYDCYITAALLIDIMNTSGWTAEQMADITGRPSLMTTFTFGKYRGKAVSDVAERDPGYLRWLFNNLDSMSPELRLTLKHYLENT

SEQ ID NO: 65 - DNA, 2748 bp, Artificial Sequence
  Made in Lab - low activity Cb1-b megaTAL DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacttaacacccgatcaagtggtagcgatagcgtcaaacggcgggggtaaacaggctttggagacggtacagcggttattgccggtactctgccaggaccacggattgacaccggaccaagtggtggcgattgcgtccaacaacggaggcaagcaggcactagagactgtccaacggcttcttcccgttctttgtcaggatcatgggctaacccctgatcaggtagtcgctatagcttcaaatggagggggcaagcaagcactggagactgttcaacgactcctgccagtgctctgccaagaccacggacttacaccagatcaagtggttgctattgcctccaatggtggcgggaaacaagcgttggaaactgtgcagagactgttacctgtcttgtgtcaagaccacggcctcacgccagatcaggtggtagccatagcgtcgaacattggtggtaagcaagcccttgaaacggtccagcgtcttctgccggtgttgtgccaggaccacggactaacgccggatcaggtcgtagccattgcttcaaatgggggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaacattggaggtaagcaagcactggagactgtccagagattgttacccgtactatgtcaagatcatggtttgacgcctgatcaggttgttgcgatagccagcaataacggagggaaacaggctcttgaaaccgtacagcgacttctcccagtcttgtgccaagatcacgggcttactcctgatcaagtcgtagctatcgccagcaataacggtggaaaacaggccctggaaaccgtacaacgtctcctcccagtactttgtcaagaccacgggttgactccggatcaagtcgtcgcgatcgcgagcaatggtggggggaagcaggcgctggaaactgttcagagactgctgcctgtactttgtcaggaccatggtctgacacctgaccaagttgtggcgatagccagtaatatcgggggaaaacaggcactagagagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcgctgatgccgaaggatgcttccgactagacatccgcaacgcaaactccatgcgtgtaaaatacagaactagactgtccttcgaaatcgtactgcacaacaaggacaaatcgattctggagaatatccagtcgacttggaaggtcggcaaaatcacaaactacggcgacagaggagtccgcctgagagtcggccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgcccccttatcaataagaacatccccaatttcaaatggctggctggattcacatctggtgacggctcgttcatggtggaactaatgaagaataagaataacgttattgtacgtgtgcgtctgagattctcaatctcccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtgtcttcagcggccgctga

SEQ ID NO: 66 - RNA, 2748 nt, Artificial Sequence
  Made in Lab - low activity Cb1-b megaTAL RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaacggcggggguaaacaggcuuuggagacgguacagcgguuauugccgguacucugccaggaccacggauugacaccggaccaagugguggcgauugcguccaacaacggaggcaagcaggcacuagagacuguccaacggcuucuucccguucuuugucaggaucaugggcuaaccccugaucagguagucgcuauagcuucaaauggagggggcaagcaagcacuggagacuguucaacgacuccugccagugcucugccaagaccacggacuuacaccagaucaagugguugcuauugccuccaaugguggcgggaaacaagcguuggaaacugugcagagacuguuaccugucuugugucaagaccacggccucacgccagaucaggugguagccauagcgucgaacauuggugguaagcaagcccuugaaacgguccagcgucuucugccgguguugugccaggaccacggacuaacgccggaucaggucguagccauugcuucaaaugggggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacauuggagguaagcaagcacuggagacuguccagagauuguuacccguacuaugucaagaucaugguuugacgccugaucagguuguugcgauagccagcaauaacggagggaaacaggcucuugaaaccguacagcgacuucucccagucuugugccaagaucacgggcuuacuccugaucaagucguagcuaucgccagcaauaacgguggaaaacaggcccuggaaaccguacaacgucuccucccaguacuuugucaagaccacggguugacuccggaucaagucgucgcgaucgcgagcaaugguggggggaagcaggcgcuggaaacuguucagagacugcugccuguacuuugucaggaccauggucugacaccugaccaaguuguggcgauagccaguaauaucgggggaaaacaggcacuagagagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucgcgcagagaguccaucaacccauggauucugacugguuucgcugaugccgaaggaugcuuccgacuagacauccgcaacgcaaacuccaugcguguaaaauacagaacuagacuguccuucgaaaucguacugcacaacaaggacaaaucgauucuggagaauauccagucgacuuggaaggucggcaaaaucacaaacuacggcgacagaggaguccgccugagagucggccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauccccaauuucaaauggcuggcuggauucacaucuggugacggcucguucaugguggaacuaaugaagaauaagaauaacguuauuguacgugugcgucugagauucucaaucucccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgugucuucagcggccgcuga

SEQ ID NO: 67 - protein, 915 aa, Artificial Sequence
  Made in Lab - low activity Cb1-b megaTAL protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGCFRLDIRNANSMRVKYRTRLSFEIVLHNKDKSILENIQSTWKVGKITNYGDRGVRLRVGRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFMVELMKNKNNVIVRVRLRFSISQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRVFSGR

SEQ ID NO: 68 - DNA, 3474 bp, Artificial Sequence
  Made in Lab - low activity Cb1-b megaTAL-Trex2 DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacttaacacccgatcaagtggtagcgatagcgtcaaacggcgggggtaaacaggctttggagacggtacagcggttattgccggtactctgccaggaccacggattgacaccggaccaagtggtggcgattgcgtccaacaacggaggcaagcaggcactagagactgtccaacggcttcttcccgttctttgtcaggatcatgggctaacccctgatcaggtagtcgctatagcttcaaatggagggggcaagcaagcactggagactgttcaacgactcctgccagtgctctgccaagaccacggacttacaccagatcaagtggttgctattgcctccaatggtggcgggaaacaagcgttggaaactgtgcagagactgttacctgtcttgtgtcaagaccacggcctcacgccagatcaggtggtagccatagcgtcgaacattggtggtaagcaagcccttgaaacggtccagcgtcttctgccggtgttgtgccaggaccacggactaacgccggatcaggtcgtagccattgcttcaaatgggggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaacattggaggtaagcaagcactggagactgtccagagattgttacccgtactatgtcaagatcatggtttgacgcctgatcaggttgttgcgatagccagcaataacggagggaaacaggctcttgaaaccgtacagcgacttctcccagtcttgtgccaagatcacgggcttactcctgatcaagtcgtagctatcgccagcaataacggtggaaaacaggccctggaaaccgtacaacgtctcctcccagtactttgtcaagaccacgggttgactccggatcaagtcgtcgcgatcgcgagcaatggtggggggaagcaggcgctggaaactgttcagagactgctgcctgtactttgtcaggaccatggtctgacacctgaccaagttgtggcgatagccagtaatatcgggggaaaacaggcactagagagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcgctgatgccgaaggatgcttccgactagacatccgcaacgcaaactccatgcgtgtaaaatacagaactagactgtccttcgaaatcgtactgcacaacaaggacaaatcgattctggagaatatccagtcgacttggaaggtcggcaaaatcacaaactacggcgacagaggagtccgcctgagagtcggccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgcccccttatcaataagaacatccccaatttcaaatggctggctggattcacatctggtgacggctcgttcatggtggaactaatgaagaataagaataacgttattgtacgtgtgcgtctgagattctcaatctcccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtggtggaggcggtagcggaggcggagggtcggctagctccgaagcaccgagagccgaaaccttcgtgttcctcgacctggaagccactggcctgcccagtgtggagcccgagattgccgagctgtccctgttcgccgttcatcggtcctccctggaaaaccctgagcacgacgagagcggagcactggtgctgccaagggtgctggacaagctgaccctgtgcatgtgtcccgagcgtcccttcactgccaaggcatccgaaatcactggtctgagttcggaagggctggctcgttgtagaaaggctggattcgatggtgccgtggttcggactttgcaggcattcctgtccaggcaggcaggtccaatctgcttggtcgcccacaacggttttgactacgacttcccactgctctgtgccgaactgcgtcgcctgggagcacgccttcctcgggataccgtgtgcctggacaccctacccgctctgcgtggactggacagagcccactcacacggcactagggcgagaggacgccagggttacagcctcggttcactgttccatcggtatttcagggcagagccttccgcagcccattcagccgaaggcgatgtccacaccttgcttctgatcttcctgcatcgtgcagcagaactgctcgcctgggcagatgagcaggctcgtggatgggctcacattgagccgatgtacttgccacctgatgaccctagcctggaagcctga

SEQ ID NO: 69 - RNA, 3474 nt, Artificial Sequence
  Made in Lab - low activity Cb1-b megaTAL-Trex2 RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaacggcggggguaaacaggcuuuggagacgguacagcgguuauugccgguacucugccaggaccacggauugacaccggaccaagugguggcgauugcguccaacaacggaggcaagcaggcacuagagacuguccaacggcuucuucccguucuuugucaggaucaugggcuaaccccugaucagguagucgcuauagcuucaaauggagggggcaagcaagcacuggagacuguucaacgacuccugccagugcucugccaagaccacggacuuacaccagaucaagugguugcuauugccuccaaugguggcgggaaacaagcguuggaaacugugcagagacuguuaccugucuugugucaagaccacggccucacgccagaucaggugguagccauagcgucgaacauuggugguaagcaagcccuugaaacgguccagcgucuucugccgguguugugccaggaccacggacuaacgccggaucaggucguagccauugcuucaaaugggggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacauuggagguaagcaagcacuggagacuguccagagauuguuacccguacuaugucaagaucaugguuugacgccugaucagguuguugcgauagccagcaauaacggagggaaacaggcucuugaaaccguacagcgacuucucccagucuugugccaagaucacgggcuuacuccugaucaagucguagcuaucgccagcaauaacgguggaaaacaggcccuggaaaccguacaacgucuccucccaguacuuugucaagaccacggguugacuccggaucaagucgucgcgaucgcgagcaaugguggggggaagcaggcgcuggaaacuguucagagacugcugccuguacuuugucaggaccauggucugacaccugaccaaguuguggcgauagccaguaauaucgggggaaaacaggcacuagagagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucgcgcagagaguccaucaacccauggauucugacugguuucgcugaugccgaaggaugcuuccgacuagacauccgcaacgcaaacuccaugcguguaaaauacagaacuagacuguccuucgaaaucguacugcacaacaaggacaaaucgauucuggagaauauccagucgacuuggaaggucggcaaaaucacaaacuacggcgacagaggaguccgccugagagucggccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauccccaauuucaaauggcuggcuggauucacaucuggugacggcucguucaugguggaacuaaugaagaauaagaauaacguuauuguacgugugcgucugagauucucaaucucccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgugguggaggcgguagcggaggcggagggucggcuagcuccgaagcaccgagagccgaaaccuucguguuccucgaccuggaagccacuggccugcccaguguggagcccgagauugccgagcugucccuguucgccguucaucgguccucccuggaaaacccugagcacgacgagagcggagcacuggugcugccaagggugcuggacaagcugacccugugcaugugucccgagcgucccuucacugccaaggcauccgaaaucacuggucugaguucggaagggcuggcucguuguagaaaggcuggauucgauggugccgugguucggacuuugcaggcauuccuguccaggcaggcagguccaaucugcuuggucgcccacaacgguuuugacuacgacuucccacugcucugugccgaacugcgucgccugggagcacgccuuccucgggauaccgugugccuggacacccuacccgcucugcguggacuggacagagcccacucacacggcacuagggcgagaggacgccaggguuacagccucgguucacuguuccaucgguauuucagggcagagccuuccgcagcccauucagccgaaggcgauguccacaccuugcuucugaucuuccugcaucgugcagcagaacugcucgccugggcagaugagcaggcucguggaugggcucacauugagccgauguacuugccaccugaugacccuagccuggaagccuga

SEQ ID NO: 70 - protein, 1157 aa, Artificial Sequence
  Made in Lab - low activity Cb1-b megaTAL-Trex2 protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGCFRLDIRNANSMRVKYRTRLSFEIVLHNKDKSILENIQSTWKVGKITNYGDRGVRLRVGRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFMVELMKNKNNVIVRVRLRFSISQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRGGGGSGGGGSASSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 71 - DNA, 3426 bp, Artificial Sequence
  Made in Lab - low activity Cb1-b megaTAL-ExoX DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacttaacacccgatcaagtggtagcgatagcgtcaaacggcgggggtaaacaggctttggagacggtacagcggttattgccggtactctgccaggaccacggattgacaccggaccaagtggtggcgattgcgtccaacaacggaggcaagcaggcactagagactgtccaacggcttcttcccgttctttgtcaggatcatgggctaacccctgatcaggtagtcgctatagcttcaaatggagggggcaagcaagcactggagactgttcaacgactcctgccagtgctctgccaagaccacggacttacaccagatcaagtggttgctattgcctccaatggtggcgggaaacaagcgttggaaactgtgcagagactgttacctgtcttgtgtcaagaccacggcctcacgccagatcaggtggtagccatagcgtcgaacattggtggtaagcaagcccttgaaacggtccagcgtcttctgccggtgttgtgccaggaccacggactaacgccggatcaggtcgtagccattgcttcaaatgggggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaacattggaggtaagcaagcactggagactgtccagagattgttacccgtactatgtcaagatcatggtttgacgcctgatcaggttgttgcgatagccagcaataacggagggaaacaggctcttgaaaccgtacagcgacttctcccagtcttgtgccaagatcacgggcttactcctgatcaagtcgtagctatcgccagcaataacggtggaaaacaggccctggaaaccgtacaacgtctcctcccagtactttgtcaagaccacgggttgactccggatcaagtcgtcgcgatcgcgagcaatggtggggggaagcaggcgctggaaactgttcagagactgctgcctgtactttgtcaggaccatggtctgacacctgaccaagttgtggcgatagccagtaatatcgggggaaaacaggcactagagagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcgctgatgccgaaggatgcttccgactagacatccgcaacgcaaactccatgcgtgtaaaatacagaactagactgtccttcgaaatcgtactgcacaacaaggacaaatcgattctggagaatatccagtcgacttggaaggtcggcaaaatcacaaactacggcgacagaggagtccgcctgagagtcggccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgcccccttatcaataagaacatccccaatttcaaatggctggctggattcacatctggtgacggctcgttcatggtggaactaatgaagaataagaataacgttattgtacgtgtgcgtctgagattctcaatctcccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtggtggaggcggtagcggaggcggagggtcggctagcctgcggatcatagacacggaaacctgtggactccagggtggtatcgtggagatagccagtgtcgatgtaattgatgggaaaatcgtaaacccgatgagccacctcgtccgaccggataggccgataagtccgcaggctatggctatacatagaatcacggaggctatggtagctgacaagccttggatcgaggacgttatcccacactactatgggagcgaatggtatgtagcgcacaacgcatcatttgataggcgcgtgcttcccgaaatgcctggggagtggatttgcacaatgaaactcgctcgccgactgtggcctggtatcaagtatagtaacatggcactttacaagacccgcaaactcaacgtacagacacctccgggtctccaccaccatagggcactctatgattgttacataaccgctgctctccttatagacataatgaatacctccgggtggaccgccgaacaaatggcagacataacgggaagacctagtttgatgacaacctttacatttggaaagtatcggggcaaggcggtgagcgatgtggccgaaagagatcccggttatcttagatggctcttcaataacttggattctatgtcacctgaacttcgccttaccctgaagcattaccttgagaatacctga

SEQ ID NO: 72 - RNA, 3426 nt, Artificial Sequence
  Made in Lab - low activity Cb1-b megaTAL-ExoX RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaacuuaacacccgaucaagugguagcgauagcgucaaacggcggggguaaacaggcuuuggagacgguacagcgguuauugccgguacucugccaggaccacggauugacaccggaccaagugguggcgauugcguccaacaacggaggcaagcaggcacuagagacuguccaacggcuucuucccguucuuugucaggaucaugggcuaaccccugaucagguagucgcuauagcuucaaauggagggggcaagcaagcacuggagacuguucaacgacuccugccagugcucugccaagaccacggacuuacaccagaucaagugguugcuauugccuccaaugguggcgggaaacaagcguuggaaacugugcagagacuguuaccugucuugugucaagaccacggccucacgccagaucaggugguagccauagcgucgaacauuggugguaagcaagcccuugaaacgguccagcgucuucugccgguguugugccaggaccacggacuaacgccggaucaggucguagccauugcuucaaaugggggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaacauuggagguaagcaagcacuggagacuguccagagauuguuacccguacuaugucaagaucaugguuugacgccugaucagguuguugcgauagccagcaauaacggagggaaacaggcucuugaaaccguacagcgacuucucccagucuugugccaagaucacgggcuuacuccugaucaagucguagcuaucgccagcaauaacgguggaaaacaggcccuggaaaccguacaacgucuccucccaguacuuugucaagaccacggguugacuccggaucaagucgucgcgaucgcgagcaaugguggggggaagcaggcgcuggaaacuguucagagacugcugccuguacuuugucaggaccauggucugacaccugaccaaguuguggcgauagccaguaauaucgggggaaaacaggcacuagagagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucgcgcagagaguccaucaacccauggauucugacugguuucgcugaugccgaaggaugcuuccgacuagacauccgcaacgcaaacuccaugcguguaaaauacagaacuagacuguccuucgaaaucguacugcacaacaaggacaaaucgauucuggagaauauccagucgacuuggaaggucggcaaaaucacaaacuacggcgacagaggaguccgccugagagucggccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauccccaauuucaaauggcuggcuggauucacaucuggugacggcucguucaugguggaacuaaugaagaauaagaauaacguuauuguacgugugcgucugagauucucaaucucccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgugguggaggcgguagcggaggcggagggucggcuagccugcggaucauagacacggaaaccuguggacuccagggugguaucguggagauagccagugucgauguaauugaugggaaaaucguaaacccgaugagccaccucguccgaccggauaggccgauaaguccgcaggcuauggcuauacauagaaucacggaggcuaugguagcugacaagccuuggaucgaggacguuaucccacacuacuaugggagcgaaugguauguagcgcacaacgcaucauuugauaggcgcgugcuucccgaaaugccuggggaguggauuugcacaaugaaacucgcucgccgacuguggccugguaucaaguauaguaacauggcacuuuacaagacccgcaaacucaacguacagacaccuccgggucuccaccaccauagggcacucuaugauuguuacauaaccgcugcucuccuuauagacauaaugaauaccuccggguggaccgccgaacaaauggcagacauaacgggaagaccuaguuugaugacaaccuuuacauuuggaaaguaucggggcaaggcggugagcgauguggccgaaagagaucccgguuaucuuagauggcucuucaauaacuuggauucuaugucaccugaacuucgccuuacccugaagcauuaccuugagaauaccuga

SEQ ID NO: 73 - protein, 1141 aa, Artificial Sequence
  Made in Lab - low activity Cb1-b megaTAL-ExoX protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALESIVAQLSRPDPALAALTNDHLVALACLGGRPAMDAVKKGLPHAPELIRRVNRRIGERTSHRVAISRVGGSSRRESINPWILTGFADAEGCFRLDIRNANSMRVKYRTRLSFEIVLHNKDKSILENIQSTWKVGKITNYGDRGVRLRVGRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFMVELMKNKNNVIVRVRLRFSISQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRGGGGSGGGGSASLRIIDTETCGLQGGIVEIASVDVIDGKIVNPMSHLVRPDRPISPQAMAIHRITEAMVADKPWIEDVIPHYYGSEWYVAHNASFDRRVLPEMPGEWICTMKLARRLWPGIKYSNMALYKTRKLNVQTPPGLHHHRALYDCYITAALLIDIMNTSGWTAEQMADITGRPSLMTTFTFGKYRGKAVSDVAERDPGYLRWLFNNLDSMSPELRLTLKHYLENT

SEQ ID NO: 74 - DNA, 2835 bp, Artificial Sequence
  Made in Lab - PD-1 megaTAL DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctaacccctgatcaggtagtcgctatagcttcaaacaacgggggcaagcaagcactggagaccgttcaacgactcctgccagtgctctgccaagaccacggacttacgccagatcaggtggttgctattgcctccaacaatggcgggaaacaagcgttggaaactgtgcagagactgttacctgtcttgtgtcaagaccacggcctcacgccagatcaggtggtagccatagcgtcgaatggaggtggtaagcaagcccttgaaacggtccagcgtcttctgccggtgttgtgccaggaccacggactaacgccggatcaggtcgtagccattgcttcaaataacggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaataatggaggtaagcaagcactggagactgtccagagattgttacccgtactatgtcaagatcatggtttgacgcctgatcaggttgttgcgatagccagcaacaacggagggaaacaggctcttgaaaccgtacagcgacttctcccagtcttgtgccaagatcacgggcttactcctgatcaagtcgtagctatcgccagccacgacggtgggaaacaggccctggaaaccgtacaacgtctcctcccagtactttgtcaagaccacgggttgactccggatcaagtcgtcgcgatcgcgagcaatggaggggggaagcaggcgctggaaactgttcagagactgctgcctgtactttgtcaggaccatggtctgacacctgaccaagttgtggcgatagccagtaacaatgggggaaaacaggcactagagacggttcaaaggttgttgcccgttctgtgccaggaccacggcttgacaccggatcaggtggtagctatcgcttcacacgatggcggaaaacaggctttagaaacagtccaaagacttctcccagtcctttgtcaggaccacggattgactccagatcaagtcgttgctattgcaagtaatggtggtggtaagcaagctttagaaaccgtacagaggcttttgccagtgctgtgccaggaccatggactgacccctgatcaagtggtagcaattgcatctcatgatggaggaaaacaagctctggaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggactctgactggtttcgctgatgccgaaggatcattcgggctaagcatcctcaacagaaacagaggtactgctagataccacactcgactgtcattcacaatcatgctgcacaacaaggacaaatcgattctggagaatatccagtcgacttggaaggtcggcagcatcctcaacaatggcgaccactacgtctcgctggtggtctaccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagaggtgattagcagggagcgcccccttatcaataagaacattccgaatgggaaatggctggctggattcacatctggtgatggctccttcttcgtgcgcctaagaaagtctaatgttaatgctagagtacgtgtgcaactggtattcgagatctcacagcacatcagagacaagaacctgatgaattcattgataacatacctaggctgtggtcacatctacgagggaaacaaatctgagcgcagttggctccaattcagagtagaaaaattcagcgatatcaacgacaagatcattccggtattccaggaaaatactctgattggcatgaaactcgaggactttgaagattggtgcaaggttgccaaattgatcgaagagaagaaacacctgaccgaatccggtttggatgagattaagaaaatcaagctgaacatgaacaaaagacgttga

SEQ ID NO: 75 - RNA, 2835 nt, Artificial Sequence
  Made in Lab - PD-1  megaTAL RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccuaaccccugaucagguagucgcuauagcuucaaacaacgggggcaagcaagcacuggagaccguucaacgacuccugccagugcucugccaagaccacggacuuacgccagaucaggugguugcuauugccuccaacaauggcgggaaacaagcguuggaaacugugcagagacuguuaccugucuugugucaagaccacggccucacgccagaucaggugguagccauagcgucgaauggaggugguaagcaagcccuugaaacgguccagcgucuucugccgguguugugccaggaccacggacuaacgccggaucaggucguagccauugcuucaaauaacggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaauaauggagguaagcaagcacuggagacuguccagagauuguuacccguacuaugucaagaucaugguuugacgccugaucagguuguugcgauagccagcaacaacggagggaaacaggcucuugaaaccguacagcgacuucucccagucuugugccaagaucacgggcuuacuccugaucaagucguagcuaucgccagccacgacggugggaaacaggcccuggaaaccguacaacgucuccucccaguacuuugucaagaccacggguugacuccggaucaagucgucgcgaucgcgagcaauggaggggggaagcaggcgcuggaaacuguucagagacugcugccuguacuuugucaggaccauggucugacaccugaccaaguuguggcgauagccaguaacaaugggggaaaacaggcacuagagacgguucaaagguuguugcccguucugugccaggaccacggcuugacaccggaucaggugguagcuaucgcuucacacgauggcggaaaacaggcuuuagaaacaguccaaagacuucucccaguccuuugucaggaccacggauugacuccagaucaagucguugcuauugcaaguaaugguggugguaagcaagcuuuagaaaccguacagaggcuuuugccagugcugugccaggaccauggacugaccccugaucaagugguagcaauugcaucucaugauggaggaaaacaagcucuggaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggacucugacugguuucgcugaugccgaaggaucauucgggcuaagcauccucaacagaaacagagguacugcuagauaccacacucgacugucauucacaaucaugcugcacaacaaggacaaaucgauucuggagaauauccagucgacuuggaaggucggcagcauccucaacaauggcgaccacuacgucucgcugguggucuaccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagaggugauuagcagggagcgcccccuuaucaauaagaacauuccgaaugggaaauggcuggcuggauucacaucuggugauggcuccuucuucgugcgccuaagaaagucuaauguuaaugcuagaguacgugugcaacugguauucgagaucucacagcacaucagagacaagaaccugaugaauucauugauaacauaccuaggcuguggucacaucuacgagggaaacaaaucugagcgcaguuggcuccaauucagaguagaaaaauucagcgauaucaacgacaagaucauuccgguauuccaggaaaauacucugauuggcaugaaacucgaggacuuugaagauuggugcaagguugccaaauugaucgaagagaagaaacaccugaccgaauccgguuuggaugagauuaagaaaaucaagcugaacaugaacaaaagacguuga

SEQ ID NO: 76 - protein, 944 aa, Artificial Sequence
  Made in Lab - PD-1  megaTAL protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAISRVGGSSRRESINPWTLTGFADAEGSFGLSILNRNRGTARYHTRLSFTIMLHNKDKSILENIQSTWKVGSILNNGDHYVSLVVYRFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRR

SEQ ID NO: 77 - DNA, 3576 bp, Artificial Sequence
  Made in Lab - PD-1 megaTAL-Trex2 DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctaacccctgatcaggtagtcgctatagcttcaaacaacgggggcaagcaagcactggagaccgttcaacgactcctgccagtgctctgccaagaccacggacttacgccagatcaggtggttgctattgcctccaacaatggcgggaaacaagcgttggaaactgtgcagagactgttacctgtcttgtgtcaagaccacggcctcacgccagatcaggtggtagccatagcgtcgaatggaggtggtaagcaagcccttgaaacggtccagcgtcttctgccggtgttgtgccaggaccacggactaacgccggatcaggtcgtagccattgcttcaaataacggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaataatggaggtaagcaagcactggagactgtccagagattgttacccgtactatgtcaagatcatggtttgacgcctgatcaggttgttgcgatagccagcaacaacggagggaaacaggctcttgaaaccgtacagcgacttctcccagtcttgtgccaagatcacgggcttactcctgatcaagtcgtagctatcgccagccacgacggtgggaaacaggccctggaaaccgtacaacgtctcctcccagtactttgtcaagaccacgggttgactccggatcaagtcgtcgcgatcgcgagcaatggaggggggaagcaggcgctggaaactgttcagagactgctgcctgtactttgtcaggaccatggtctgacacctgaccaagttgtggcgatagccagtaacaatgggggaaaacaggcactagagacggttcaaaggttgttgcccgttctgtgccaggaccacggcttgacaccggatcaggtggtagctatcgcttcacacgatggcggaaaacaggctttagaaacagtccaaagacttctcccagtcctttgtcaggaccacggattgactccagatcaagtcgttgctattgcaagtaatggtggtggtaagcaagctttagaaaccgtacagaggcttttgccagtgctgtgccaggaccatggactgacccctgatcaagtggtagcaattgcatctcatgatggaggaaaacaagctctggaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggactctgactggtttcgctgatgccgaaggatcattcgggctaagcatcctcaacagaaacagaggtactgctagataccacactcgactgtcattcacaatcatgctgcacaacaaggacaaatcgattctggagaatatccagtcgacttggaaggtcggcagcatcctcaacaatggcgaccactacgtctcgctggtggtctaccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagaggtgattagcagggagcgcccccttatcaataagaacattccgaatgggaaatggctggctggattcacatctggtgatggctccttcttcgtgcgcctaagaaagtctaatgttaatgctagagtacgtgtgcaactggtattcgagatctcacagcacatcagagacaagaacctgatgaattcattgataacatacctaggctgtggtcacatctacgagggaaacaaatctgagcgcagttggctccaattcagagtagaaaaattcagcgatatcaacgacaagatcattccggtattccaggaaaatactctgattggcatgaaactcgaggactttgaagattggtgcaaggttgccaaattgatcgaagagaagaaacacctgaccgaatccggtttggatgagattaagaaaatcaagctgaacatgaacaaaagacgtggtggaggcggtagcggaggcggagggtcggctagctccgaagcaccgagagccgaaaccttcgtgttcctcgacctggaagccactggcctgcccagtgtggagcccgagattgccgagctgtccctgttcgccgttcatcggtcctccctggaaaaccctgagcacgacgagagcggagcactggtgctgccaagggtgctggacaagctgaccctgtgcatgtgtcccgagcgtcccttcactgccaaggcatccgaaatcactggtctgagttcggaagggctggctcgttgtagaaaggctggattcgatggtgccgtggttcggactttgcaggcattcctgtccaggcaggcaggtccaatctgcttggtcgcccacaacggttttgactacgacttcccactgctctgtgccgaactgcgtcgcctgggagcacgccttcctcgggataccgtgtgcctggacaccctacccgctctgcgtggactggacagagcccactcacacggcactagggcgagaggacgccagggttacagcctcggttcactgttccatcggtatttcagggcagagccttccgcagcccattcagccgaaggcgatgtccacaccttgcttctgatcttcctgcatcgtgcagcagaactgctcgcctgggcagatgagcaggctcgtggatgggctcacattgagccgatgtacttgccacctgatgaccctagcctggaagcctga

SEQ ID NO: 78 - RNA, 3576 nt, Artificial Sequence
  Made in Lab - PD-1 megaTAL-Trex2 RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccuaaccccugaucagguagucgcuauagcuucaaacaacgggggcaagcaagcacuggagaccguucaacgacuccugccagugcucugccaagaccacggacuuacgccagaucaggugguugcuauugccuccaacaauggcgggaaacaagcguuggaaacugugcagagacuguuaccugucuugugucaagaccacggccucacgccagaucaggugguagccauagcgucgaauggaggugguaagcaagcccuugaaacgguccagcgucuucugccgguguugugccaggaccacggacuaacgccggaucaggucguagccauugcuucaaauaacggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaauaauggagguaagcaagcacuggagacuguccagagauuguuacccguacuaugucaagaucaugguuugacgccugaucagguuguugcgauagccagcaacaacggagggaaacaggcucuugaaaccguacagcgacuucucccagucuugugccaagaucacgggcuuacuccugaucaagucguagcuaucgccagccacgacggugggaaacaggcccuggaaaccguacaacgucuccucccaguacuuugucaagaccacggguugacuccggaucaagucgucgcgaucgcgagcaauggaggggggaagcaggcgcuggaaacuguucagagacugcugccuguacuuugucaggaccauggucugacaccugaccaaguuguggcgauagccaguaacaaugggggaaaacaggcacuagagacgguucaaagguuguugcccguucugugccaggaccacggcuugacaccggaucaggugguagcuaucgcuucacacgauggcggaaaacaggcuuuagaaacaguccaaagacuucucccaguccuuugucaggaccacggauugacuccagaucaagucguugcuauugcaaguaaugguggugguaagcaagcuuuagaaaccguacagaggcuuuugccagugcugugccaggaccauggacugaccccugaucaagugguagcaauugcaucucaugauggaggaaaacaagcucuggaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggacucugacugguuucgcugaugccgaaggaucauucgggcuaagcauccucaacagaaacagagguacugcuagauaccacacucgacugucauucacaaucaugcugcacaacaaggacaaaucgauucuggagaauauccagucgacuuggaaggucggcagcauccucaacaauggcgaccacuacgucucgcugguggucuaccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagaggugauuagcagggagcgcccccuuaucaauaagaacauuccgaaugggaaauggcuggcuggauucacaucuggugauggcuccuucuucgugcgccuaagaaagucuaauguuaaugcuagaguacgugugcaacugguauucgagaucucacagcacaucagagacaagaaccugaugaauucauugauaacauaccuaggcuguggucacaucuacgagggaaacaaaucugagcgcaguuggcuccaauucagaguagaaaaauucagcgauaucaacgacaagaucauuccgguauuccaggaaaauacucugauuggcaugaaacucgaggacuuugaagauuggugcaagguugccaaauugaucgaagagaagaaacaccugaccgaauccgguuuggaugagauuaagaaaaucaagcugaacaugaacaaaagacgugguggaggcgguagcggaggcggagggucggcuagcuccgaagcaccgagagccgaaaccuucguguuccucgaccuggaagccacuggccugcccaguguggagcccgagauugccgagcugucccuguucgccguucaucgguccucccuggaaaacccugagcacgacgagagcggagcacuggugcugccaagggugcuggacaagcugacccugugcaugugucccgagcgucccuucacugccaaggcauccgaaaucacuggucugaguucggaagggcuggcucguuguagaaaggcuggauucgauggugccgugguucggacuuugcaggcauuccuguccaggcaggcagguccaaucugcuuggucgcccacaacgguuuugacuacgacuucccacugcucugugccgaacugcgucgccugggagcacgccuuccucgggauaccgugugccuggacacccuacccgcucugcguggacuggacagagcccacucacacggcacuagggcgagaggacgccaggguuacagccucgguucacuguuccaucgguauuucagggcagagccuuccgcagcccauucagccgaaggcgauguccacaccuugcuucugaucuuccugcaucgugcagcagaacugcucgccugggcagaugagcaggcucguggaugggcucacauugagccgauguacuugccaccugaugacccuagccuggaagccuga

SEQ ID NO: 79 - protein, 1191 aa, Artificial Sequence
  Made in Lab - PD-1 megaTAL-Trex2 protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAISRVGGSSRRESINPWTLTGFADAEGSFGLSILNRNRGTARYHTRLSFTIMLHNKDKSILENIQSTWKVGSILNNGDHYVSLVVYRFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRRGGGGSGGGGSASSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 80 - DNA, 3528 bp, Artificial Sequence
  Made in Lab - PD-1 megaTAL-ExoX DNA construct
atgggatcctgcaggccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctaacccctgatcaggtagtcgctatagcttcaaacaacgggggcaagcaagcactggagaccgttcaacgactcctgccagtgctctgccaagaccacggacttacgccagatcaggtggttgctattgcctccaacaatggcgggaaacaagcgttggaaactgtgcagagactgttacctgtcttgtgtcaagaccacggcctcacgccagatcaggtggtagccatagcgtcgaatggaggtggtaagcaagcccttgaaacggtccagcgtcttctgccggtgttgtgccaggaccacggactaacgccggatcaggtcgtagccattgcttcaaataacggcggcaaacaggcgctagagacagtccagcgcctcttgcctgtgttatgccaggatcacggcttaaccccagaccaagttgtggctattgcatctaacaatggtggcaaacaagccttggagacagtgcaacgattactgcctgtcttatgtcaggatcatggcctgacgcccgatcaggtagtggcaatcgcatctaataatggaggtaagcaagcactggagactgtccagagattgttacccgtactatgtcaagatcatggtttgacgcctgatcaggttgttgcgatagccagcaacaacggagggaaacaggctcttgaaaccgtacagcgacttctcccagtcttgtgccaagatcacgggcttactcctgatcaagtcgtagctatcgccagccacgacggtgggaaacaggccctggaaaccgtacaacgtctcctcccagtactttgtcaagaccacgggttgactccggatcaagtcgtcgcgatcgcgagcaatggaggggggaagcaggcgctggaaactgttcagagactgctgcctgtactttgtcaggaccatggtctgacacctgaccaagttgtggcgatagccagtaacaatgggggaaaacaggcactagagacggttcaaaggttgttgcccgttctgtgccaggaccacggcttgacaccggatcaggtggtagctatcgcttcacacgatggcggaaaacaggctttagaaacagtccaaagacttctcccagtcctttgtcaggaccacggattgactccagatcaagtcgttgctattgcaagtaatggtggtggtaagcaagctttagaaaccgtacagaggcttttgccagtgctgtgccaggaccatggactgacccctgatcaagtggtagcaattgcatctcatgatggaggaaaacaagctctggaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctctcgcagagagtccatcaacccatggactctgactggtttcgctgatgccgaaggatcattcgggctaagcatcctcaacagaaacagaggtactgctagataccacactcgactgtcattcacaatcatgctgcacaacaaggacaaatcgattctggagaatatccagtcgacttggaaggtcggcagcatcctcaacaatggcgaccactacgtctcgctggtggtctaccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagaggtgattagcagggagcgcccccttatcaataagaacattccgaatgggaaatggctggctggattcacatctggtgatggctccttcttcgtgcgcctaagaaagtctaatgttaatgctagagtacgtgtgcaactggtattcgagatctcacagcacatcagagacaagaacctgatgaattcattgataacatacctaggctgtggtcacatctacgagggaaacaaatctgagcgcagttggctccaattcagagtagaaaaattcagcgatatcaacgacaagatcattccggtattccaggaaaatactctgattggcatgaaactcgaggactttgaagattggtgcaaggttgccaaattgatcgaagagaagaaacacctgaccgaatccggtttggatgagattaagaaaatcaagctgaacatgaacaaaagacgtggtggaggcggtagcggaggcggagggtcggctagcctgcggatcatagacacggaaacctgtggactccagggtggtatcgtggagatagccagtgtcgatgtaattgatgggaaaatcgtaaacccgatgagccacctcgtccgaccggataggccgataagtccgcaggctatggctatacatagaatcacggaggctatggtagctgacaagccttggatcgaggacgttatcccacactactatgggagcgaatggtatgtagcgcacaacgcatcatttgataggcgcgtgcttcccgaaatgcctggggagtggatttgcacaatgaaactcgctcgccgactgtggcctggtatcaagtatagtaacatggcactttacaagacccgcaaactcaacgtacagacacctccgggtctccaccaccatagggcactctatgattgttacataaccgctgctctccttatagacataatgaatacctccgggtggaccgccgaacaaatggcagacataacgggaagacctagtttgatgacaacctttacatttggaaagtatcggggcaaggcggtgagcgatgtggccgaaagagatcccggttatcttagatggctcttcaataacttggattctatgtcacctgaacttcgccttaccctgaagcattaccttgagaatacctga

SEQ ID NO: 81 - RNA, 3528 nt, Artificial Sequence
  Made in Lab - PD-1 megaTAL-ExoX RNA construct
augggauccugcaggccaccuaagaagaaacgcaaagucguggaucuacgcacgcucggcuacagucagcagcagcaagagaagaucaaaccgaaggugcguucgacaguggcgcagcaccacgaggcacuggugggccauggguuuacacacgcgcacaucguugcgcucagccaacacccggcagcguuagggaccgucgcugucacguaucagcacauaaucacggcguugccagaggcgacacacgaagacaucguuggcgucggcaaacagugguccggcgcacgcgcccuggaggccuugcucacggaugcgggggaguugagagguccgccguuacaguuggacacaggccaacuugugaagauugcaaaacguggcggcgugaccgcaauggaggcagugcaugcaucgcgcaaugcacugacgggugccccccugaaccuaaccccugaucagguagucgcuauagcuucaaacaacgggggcaagcaagcacuggagaccguucaacgacuccugccagugcucugccaagaccacggacuuacgccagaucaggugguugcuauugccuccaacaauggcgggaaacaagcguuggaaacugugcagagacuguuaccugucuugugucaagaccacggccucacgccagaucaggugguagccauagcgucgaauggaggugguaagcaagcccuugaaacgguccagcgucuucugccgguguugugccaggaccacggacuaacgccggaucaggucguagccauugcuucaaauaacggcggcaaacaggcgcuagagacaguccagcgccucuugccuguguuaugccaggaucacggcuuaaccccagaccaaguuguggcuauugcaucuaacaaugguggcaaacaagccuuggagacagugcaacgauuacugccugucuuaugucaggaucauggccugacgcccgaucagguaguggcaaucgcaucuaauaauggagguaagcaagcacuggagacuguccagagauuguuacccguacuaugucaagaucaugguuugacgccugaucagguuguugcgauagccagcaacaacggagggaaacaggcucuugaaaccguacagcgacuucucccagucuugugccaagaucacgggcuuacuccugaucaagucguagcuaucgccagccacgacggugggaaacaggcccuggaaaccguacaacgucuccucccaguacuuugucaagaccacggguugacuccggaucaagucgucgcgaucgcgagcaauggaggggggaagcaggcgcuggaaacuguucagagacugcugccuguacuuugucaggaccauggucugacaccugaccaaguuguggcgauagccaguaacaaugggggaaaacaggcacuagagacgguucaaagguuguugcccguucugugccaggaccacggcuugacaccggaucaggugguagcuaucgcuucacacgauggcggaaaacaggcuuuagaaacaguccaaagacuucucccaguccuuugucaggaccacggauugacuccagaucaagucguugcuauugcaaguaaugguggugguaagcaagcuuuagaaaccguacagaggcuuuugccagugcugugccaggaccauggacugaccccugaucaagugguagcaauugcaucucaugauggaggaaaacaagcucuggaaagcauuguggcccagcugagccggccugauccggcguuggccgcguugaccaacgaccaccucgucgccuuggccugccucggcggacguccugccauggaugcagugaaaaagggauugccgcacgcgccggaauugaucagaagagucaaucgccguauuggcgaacgcacgucccaucgcguugcgauaucuagagugggaggaagcucucgcagagaguccaucaacccauggacucugacugguuucgcugaugccgaaggaucauucgggcuaagcauccucaacagaaacagagguacugcuagauaccacacucgacugucauucacaaucaugcugcacaacaaggacaaaucgauucuggagaauauccagucgacuuggaaggucggcagcauccucaacaauggcgaccacuacgucucgcugguggucuaccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagaggugauuagcagggagcgcccccuuaucaauaagaacauuccgaaugggaaauggcuggcuggauucacaucuggugauggcuccuucuucgugcgccuaagaaagucuaauguuaaugcuagaguacgugugcaacugguauucgagaucucacagcacaucagagacaagaaccugaugaauucauugauaacauaccuaggcuguggucacaucuacgagggaaacaaaucugagcgcaguuggcuccaauucagaguagaaaaauucagcgauaucaacgacaagaucauuccgguauuccaggaaaauacucugauuggcaugaaacucgaggacuuugaagauuggugcaagguugccaaauugaucgaagagaagaaacaccugaccgaauccgguuuggaugagauuaagaaaaucaagcugaacaugaacaaaagacgugguggaggcgguagcggaggcggagggucggcuagccugcggaucauagacacggaaaccuguggacuccagggugguaucguggagauagccagugucgauguaauugaugggaaaaucguaaacccgaugagccaccucguccgaccggauaggccgauaaguccgcaggcuauggcuauacauagaaucacggaggcuaugguagcugacaagccuuggaucgaggacguuaucccacacuacuaugggagcgaaugguauguagcgcacaacgcaucauuugauaggcgcgugcuucccgaaaugccuggggaguggauuugcacaaugaaacucgcucgccgacuguggccugguaucaaguauaguaacauggcacuuuacaagacccgcaaacucaacguacagacaccuccgggucuccaccaccauagggcacucuaugauuguuacauaaccgcugcucuccuuauagacauaaugaauaccuccggguggaccgccgaacaaauggcagacauaacgggaagaccuaguuugaugacaaccuuuacauuuggaaaguaucggggcaaggcggugagcgauguggccgaaagagaucccgguuaucuuagauggcucuucaauaacuuggauucuaugucaccugaacuucgccuuacccugaagcauuaccuugagaauaccuga

SEQ ID NO: 82 - protein, 1175 aa, Artificial Sequence
  Made in Lab - PD-1 megaTAL-ExoX protein construct
MGSCRPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALESIVAQLSRPDPALAALTNDHLVALACLGGRPAMDAVKKGLPHAPELIRRVNRRIGERTSHRVAISRVGGSSRRESINPWTLTGFADAEGSFGLSILNRNRGTARYHTRLSFTIMLHNKDKSILENIQSTWKVGSILNNGDHYVSLVVYRFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRRGGGGSGGGGSASLRIIDTETCGLQGGIVEIASVDVIDGKIVNPMSHLVRPDRPISPQAMAIHRITEAMVADKPWIEDVIPHYYGSEWYVAHNASFDRRVLPEMPGEWICTMKLARRLWPGIKYSNMALYKTRKLNVQTPPGLHHHRALYDCYITAALLIDIMNTSGWTAEQMADITGRPSLMTTFTFGKYRGKAVSDVAERDPGYLRWLFNNLDSMSPELRLTLKHYLENT

SEQ ID NO: 83 - RNA, 870 nt, Homo sapiens
augguccagaucccacaggcgcccuggccagucgucugggcggugcuacaacugggcuggcggccaggaugguucuuagacuccccagacaggcccuggaacccccccaccuucuccccagcccugcucguggugaccgaaggggacaacgccaccuucaccugcagcuucuccaacacaucggagagcuucgugcuaaacugguaccgcaugagccccagcaaccagacggacaagcuggccgccuuccccgaggaccgcagccagcccggccaggacugccgcuuccgugucacacaacugcccaacgggcgugacuuccacaugagcguggucagggcccggcgcaaugacagcggcaccuaccucuguggggccaucucccuggcccccaaggcgcagaucaaagagagccugcgggcagagcucagggugacagagagaagggcagaagugcccacagcccaccccagccccucacccaggccagccggccaguuccaaacccuggugguuggugucgugggcggccugcugggcagccuggugcugcuagucuggguccuggccgucaucugcucccgggccgcacgagggacaauaggagccaggcgcaccggccagccccugaaggaggaccccucagccgugccuguguucucuguggacuauggggagcuggauuuccaguggcgagagaagaccccggagccccccgugcccugugucccugagcagacggaguaugccaccauugucuuuccuagcggaaugggcaccucaucccccgcccgcaggggcucagcugacggcccucggagugcccagccacugaggccugaggauggacacugcucuuggccccucuga

SEQ ID NO: 84 - RNA, 869 nt, Artificial sequence
  mock-edited PD-1 open reading frame with deletion
augguccagacccacaggcgcccuggccagucgucugggcggugcuacaacugggcuggcggccaggaugguucuuagacuccccagacaggcccuggaacccccccaccuucuccccagcccugcucguggugaccgaaggggacaacgccaccuucaccugcagcuucuccaacacaucggagagcuucgugcuaaacugguaccgcaugagccccagcaaccagacggacaagcuggccgccuuccccgaggaccgcagccagcccggccaggacugccgcuuccgugucacacaacugcccaacgggcgugacuuccacaugagcguggucagggcccggcgcaaugacagcggcaccuaccucuguggggccaucucccuggcccccaaggcgcagaucaaagagagccugcgggcagagcucagggugacagagagaagggcagaagugcccacagcccaccccagccccucacccaggccagccggccaguuccaaacccuggugguuggugucgugggcggccugcugggcagccuggugcugcuagucuggguccuggccgucaucugcucccgggccgcacgagggacaauaggagccaggcgcaccggccagccccugaaggaggaccccucagccgugccuguguucucuguggacuauggggagcuggauuuccaguggcgagagaagaccccggagccccccgugcccugugucccugagcagacggaguaugccaccauugucuuuccuagcggaaugggcaccucaucccccgcccgcaggggcucagcugacggcccucggagugcccagccacugaggccugaggauggacacugcucuuggccccucuga

SEQ ID NO: 85 - RNA, 868 nt, Artificial Sequence
  mock-edited PD-1 open reading frame with deletion
augguccagaccacaggcgcccuggccagucgucugggcggugcuacaacugggcuggcggccaggaugguucuuagacuccccagacaggcccuggaacccccccaccuucuccccagcccugcucguggugaccgaaggggacaacgccaccuucaccugcagcuucuccaacacaucggagagcuucgugcuaaacugguaccgcaugagccccagcaaccagacggacaagcuggccgccuuccccgaggaccgcagccagcccggccaggacugccgcuuccgugucacacaacugcccaacgggcgugacuuccacaugagcguggucagggcccggcgcaaugacagcggcaccuaccucuguggggccaucucccuggcccccaaggcgcagaucaaagagagccugcgggcagagcucagggugacagagagaagggcagaagugcccacagcccaccccagccccucacccaggccagccggccaguuccaaacccuggugguuggugucgugggcggccugcugggcagccuggugcugcuagucuggguccuggccgucaucugcucccgggccgcacgagggacaauaggagccaggcgcaccggccagccccugaaggaggaccccucagccgugccuguguucucuguggacuauggggagcuggauuuccaguggcgagagaagaccccggagccccccgugcccugugucccugagcagacggaguaugccaccauugucuuuccuagcggaaugggcaccucaucccccgcccgcaggggcucagcugacggcccucggagugcccagccacugaggccugaggauggacacugcucuuggccccucuga

SEQ ID NO: 86 - RNA, 867 nt, Artificial Sequence
  mock-edited PD-1 open reading frame with deletion
augguccagccacaggcgcccuggccagucgucugggcggugcuacaacugggcuggcggccaggaugguucuuagacuccccagacaggcccuggaacccccccaccuucuccccagcccugcucguggugaccgaaggggacaacgccaccuucaccugcagcuucuccaacacaucggagagcuucgugcuaaacugguaccgcaugagccccagcaaccagacggacaagcuggccgccuuccccgaggaccgcagccagcccggccaggacugccgcuuccgugucacacaacugcccaacgggcgugacuuccacaugagcguggucagggcccggcgcaaugacagcggcaccuaccucuguggggccaucucccuggcccccaaggcgcagaucaaagagagccugcgggcagagcucagggugacagagagaagggcagaagugcccacagcccaccccagccccucacccaggccagccggccaguuccaaacccuggugguuggugucgugggcggccugcugggcagccuggugcugcuagucuggguccuggccgucaucugcucccgggccgcacgagggacaauaggagccaggcgcaccggccagccccugaaggaggaccccucagccgugccuguguucucuguggacuauggggagcuggauuuccaguggcgagagaagaccccggagccccccgugcccugugucccugagcagacggaguaugccaccauugucuuuccuagcggaaugggcaccucaucccccgcccgcaggggcucagcugacggcccucggagugcccagccacugaggccugaggauggacacugcucuuggccccucuga

SEQ ID NO: 87 - DNA, 891 bp, Artificial Sequence
  Engineered variant of I-OnuI LHE
tctcgcagagagtccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgaaggctacttcggcgtgaatctaaaaaaggtaaagggcaccgcaaaggtatacgtgggactgagattctcaatctcacagcacatcagagacaagaacctgatgaattcattgataacatacctaggctgtggttccatctgggagaagaacaagtctgagttcagatggctcgagttcgtcgtaaccaaattcagcgatatcaacgacaagatcattccggtattccaggaaaatactctgattggcgtcaaactcgaggactttgaagattggtgcaaggttgccaaattgatcaaagagaagaaacacctgaccgaatccggtttggatgagattaagaaaatcaagctgaacatgaacaaaggtcgt

SEQ ID NO: 88 - RNA, 891 nt, Artificial Sequence
  Engineered variant of I-OnuI LHE
ucucgcagagaguccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugaaggcuacuucggcgugaaucuaaaaaagguaaagggcaccgcaaagguauacgugggacugagauucucaaucucacagcacaucagagacaagaaccugaugaauucauugauaacauaccuaggcugugguuccaucugggagaagaacaagucugaguucagauggcucgaguucgucguaaccaaauucagcgauaucaacgacaagaucauuccgguauuccaggaaaauacucugauuggcgucaaacucgaggacuuugaagauuggugcaagguugccaaauugaucaaagagaagaaacaccugaccgaauccgguuuggaugagauuaagaaaaucaagcugaacaugaacaaaggucgu

SEQ ID NO: 89 - protein, 303 aa, Artificial Sequence
  Engineered variant of I-OnuI LHE
  MOD_RES (1)..(1): Xaa is any amino acid or absent
  MOD_RES (2)..(2): Xaa is any amino acid or absent
  MOD_RES (3)..(3): Xaa is any amino acid or absent
  MOD_RES (4)..(4): Xaa is any amino acid or absent
  MOD_RES (302)..(302): Xaa is any amino acid or absent
  MOD_RES (303)..(303): Xaa is any amino acid or absent
XXXXSRRESINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGEGYFGVNLKKVKGTAKVYVGLRFSISQHIRDKNLMNSLITYLGCGSIWEKNKSEFRWLEFVVTKFSDINDKIIPVFQENTLIGVKLEDFEDWCKVAKLIKEKKHLTESGLDEIKKIKLNMNKGRXX

SEQ ID NO: 90 - DNA, 882 bp, Artificial Sequence
  Engineered variant of I-OnuI LHE
tccatcaacccatggattctgactggtttcgctgatgccgaaggatcattcatactagacatccgcaaccgaaacaacgaaagcaacagataccgaacttcgctgagattccagatcaccctgcacaacaaggacaaatcgattctggagaatatccagtccacttggaaggtcggcaagatcacaaacagcagtgacagagccgtcatgctgagggtcacccgtttcgaagatttgaaagtgattatcgaccacttcgagaaatatccgctgattacccagaaattgggcgattacaagttgtttaaacaggcattcagcgtcatggagaataaagaacatcttaaggagaatgggattaaggagctcgtacgaatcaaagctaagatgaattggggtctcaatgacgaattgaaaaaagcatttccagagaacattagcaaagagcgcccccttatcaataagaacattccgaatttcaaatggctggctggattcacagctggtgatggccatttcggcgtgaatctaaaaaaggtaaagggcaccgcaaaggtatacgtgggactgagattcgctatctcacagcacatcagagacaagaacctgatgaattcattgataacatacctaggctgtggttccatcagagagaagaacaagtctgagttcagatggctcgagttcgaagtaaccaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgttga

SEQ ID NO: 91 - RNA, 882 nt, Artificial Sequence
  Engineered variant of I-OnuI LHE
uccaucaacccauggauucugacugguuucgcugaugccgaaggaucauucauacuagacauccgcaaccgaaacaacgaaagcaacagauaccgaacuucgcugagauuccagaucacccugcacaacaaggacaaaucgauucuggagaauauccaguccacuuggaaggucggcaagaucacaaacagcagugacagagccgucaugcugagggucacccguuucgaagauuugaaagugauuaucgaccacuucgagaaauauccgcugauuacccagaaauugggcgauuacaaguuguuuaaacaggcauucagcgucauggagaauaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauuccgaauuucaaauggcuggcuggauucacagcuggugauggccauuucggcgugaaucuaaaaaagguaaagggcaccgcaaagguauacgugggacugagauucgcuaucucacagcacaucagagacaagaaccugaugaauucauugauaacauaccuaggcugugguuccaucagagagaagaacaagucugaguucagauggcucgaguucgaaguaaccaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucguuga

SEQ ID NO: 92 - protein, 303 aa, Artificial Sequence
  Engineered variant of I-OnuI LHE
  MOD_RES (1)..(1): Xaa is any amino acid or absent
  MOD_RES (2)..(2): Xaa is any amino acid or absent
  MOD_RES (3)..(3): Xaa is any amino acid or absent
  MOD_RES (4)..(4): Xaa is any amino acid or absent
  MOD_RES (5)..(5): Xaa is any amino acid or absent
  MOD_RES (6)..(6): Xaa is any amino acid or absent
  MOD_RES (7)..(7): Xaa is any amino acid or absent
  MOD_RES (8)..(8): Xaa is any amino acid or absent
  MOD_RES (302)..(302): Xaa is any amino acid or absent
  MOD_RES (303)..(303): Xaa is any amino acid or absent
XXXXXXXXSINPWILTGFADAEGSFILDIRNRNNESNRYRTSLRFQITLHNKDKSILENIQSTWKVGKITNSSDRAVMLRVTRFEDLKVIIDHFEKYPLITQKLGDYKLFKQAFSVMENKEHLKENGIKELVRIKAKMNWGLNDELKKAFPENISKERPLINKNIPNFKWLAGFTAGDGHFGVNLKKVKGTAKVYVGLRFAISQHIRDKNLMNSLITYLGCGSIREKNKSEFRWLEFEVTKFSDINDKIIPVFQENTLIGVKLEDFEDWCKVAKLIEEKKHLTESGLDEIKKIKLNMNKGRXX

SEQ ID NO: 93 - DNA, 891 bp, Artificial Sequence
  Engineered variant of I-OnuI LHE
tcgcgcagagagtccatcaacccatggattctgactggtttcgctgatgccgaaggatgcttccgactagacatccgcaacgcaaactccatgcgtgtaaaatacagaactagactgtccttcgaaatcgtactgcacaacaaggacaaatcgattctggagaatatccagtcgacttggaaggtcggcaaaatcacaaactacggcgacagaggagtccgcctgagagtcggccgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgcccccttatcaataagaacatccccaatttcaaatggctggctggattcacatctggtgacggctcgttcatggtggaactaatgaagaataagaataacgttattgtacgtgtgcgtctgagattctcaatctcccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgt

SEQ ID NO: 94 - RNA, 891 nt, Artificial Sequence
  Engineered variant of I-OnuI LHE
ucgcgcagagaguccaucaacccauggauucugacugguuucgcugaugccgaaggaugcuuccgacuagacauccgcaacgcaaacuccaugcguguaaaauacagaacuagacuguccuucgaaaucguacugcacaacaaggacaaaucgauucuggagaauauccagucgacuuggaaggucggcaaaaucacaaacuacggcgacagaggaguccgccugagagucggccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcccccuuaucaauaagaacauccccaauuucaaauggcuggcuggauucacaucuggugacggcucguucaugguggaacuaaugaagaauaagaauaacguuauuguacgugugcgucugagauucucaaucucccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgu

SEQ ID NO: 95 - protein, 303 aa, Artificial Sequence
  Engineered variant of I-OnuI LHE
  MOD_RES (1)..(1): Xaa is any amino acid or absent
  MOD_RES (2)..(2): Xaa is any amino acid or absent
  MOD_RES (3)..(3): Xaa is any amino acid or absent
  MOD_RES (4)..(4): Xaa is any amino acid or absent
  MOD_RES (302)..(302): Xaa is any amino acid or absent
  MOD_RES (303)..(303): Xaa is any amino acid or absent
XXXXSRRESINPWILTGFADAEGCFRLDIRNANSMRVKYRTRLSFEIVLHNKDKSILENIQSTWKVGKITNYGDRGVRLRVGRFEDLKVIIDHFEKYPLITQKLGDYKLFKQAFSVMENKEHLKENGIKELVRIKAKMNWGLNDELKKAFPENISKERPLINKNIPNFKWLAGFTSGDGSFMVELMKNKNNVIVRVRLRFSISQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRXX

SEQ ID NO: 96 - DNA, 891 bp, Artificial Sequence
  Engineered variant of I-OnuI LHE
tcgcgcagagagtccatcaacccatggattctgactggtttcgctgatgccgaaggatgcttccgactagacatccgcaacgcaaacgatttaagagccggatacagaactagactggccttcgaaatcgtactgcacaacaaggacaaatcgattctggagaatatccagtcgacttggaaggtcggcacaatctacaacgcgggcgacaacgcagtcagactgcaagtcacacgtttcgaagatttgaaagtgattatcgaccacttcgagaaatatccgctgataacacagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacattagcaaagagcgctcccttatcaataagaacattccgaatctcaaatggctggctggattcacatctggtgacggctcgttcgtggtggaactaaagaagagaagaagccccgtcaaggtaggagtgcggctgcgattcagcatcacccagcacatcagagacaagaacctgatgaattcattgataacatacctaggctgtggtcgtatcgttgagaataacaaatctgagcacagttggctcgaattcattgtaacaaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgt

SEQ ID NO: 97 - RNA, 891 nt, Artificial Sequence
  Engineered variant of I-OnuI LHE
ucgcgcagagaguccaucaacccauggauucugacugguuucgcugaugccgaaggaugcuuccgacuagacauccgcaacgcaaacgauuuaagagccggauacagaacuagacuggccuucgaaaucguacugcacaacaaggacaaaucgauucuggagaauauccagucgacuuggaaggucggcacaaucuacaacgcgggcgacaacgcagucagacugcaagucacacguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagagaacauuagcaaagagcgcucccuuaucaauaagaacauuccgaaucucaaauggcuggcuggauucacaucuggugacggcucguucgugguggaacuaaagaagagaagaagccccgucaagguaggagugcggcugcgauucagcaucacccagcacaucagagacaagaaccugaugaauucauugauaacauaccuaggcuguggucguaucguugagaauaacaaaucugagcacaguuggcucgaauucauuguaacaaaauucagcgauaucaacgacaagaucauuccgguauuccaggaaaauacucugauuggcgucaaacucgaggacuuugaagauuggugcaagguugccaaauugaucgaagagaagaaacaccugaccgaauccgguuuggaugagauuaagaaaaucaagcugaacaugaacaaaggucgu

SEQ ID NO: 98 - protein, 303 aa, Artificial Sequence
  Engineered variant of I-OnuI LHE
  MOD_RES (1)..(1): Xaa is any amino acid or absent
  MOD_RES (2)..(2): Xaa is any amino acid or absent
  MOD_RES (3)..(3): Xaa is any amino acid or absent
  MOD_RES (4)..(4): Xaa is any amino acid or absent
  MOD_RES (302)..(302): Xaa is any amino acid or absent
  MOD_RES (303)..(303): Xaa is any amino acid or absent
XXXXSRRESINPWILTGFADAEGCFRLDIRNANDLRAGYRTRLAFEIVLHNKDKSILENIQSTWKVGTIYNAGDNAVRLQVTRFEDLKVIIDHFEKYPLITQKLGDYKLFKQAFSVMENKEHLKENGIKELVRIKAKMNWGLNDELKKAFPENISKERSLINKNIPNLKWLAGFTSGDGSFVVELKKRRSPVKVGVRLRFSITQHIRDKNLMNSLITYLGCGRIVENNKSEHSWLEFIVTKFSDINDKIIPVFQENTLIGVKLEDFEDWCKVAKLIEEKKHLTESGLDEIKKIKLNMNKGRXX

SEQ ID NO: 99 - DNA, 443 bp, Artificial Sequence
  Engineered variant of I-OnuI LHE
ttagcagggagcgcccccttatcaataagaacattccgaatgggaaatggctggctggattcacatctggtgatggctccttcttcgtgcgcctaagaaagtctaatgttaatgctagagtacgtgtgcaactggtattcgagatctcacagcacatcagagacaagaacctgatgaattcattgataacatacctaggctgtggtcacatctacgagggaaacaaatctgagcgcagttggctccaattcagagtagaaaaattcagcgatatcaacgacaagatcattccggtattccaggaaaatactctgattggcatgaaactcgaggactttgaagattggtgcaaggttgccaaattgatcgaagagaagaaacacctgaccgaatccggtttggatgagattaagaaaatcaagctgaacatgaacaaaagacgt

SEQ ID NO: 100 - RNA, 891 nt, Artificial Sequence
  Engineered variant of I-OnuI LHE
ucucgcagagaguccaucaacccauggacucugacugguuucgcugaugccgaaggaucauucgggcuaagcauccucaacagaaacagagguacugcuagauaccacacucgacugucauucacaaucaugcugcacaacaaggacaaaucgauucuggagaauauccagucgacuuggaaggucggcagcauccucaacaauggcgaccacuacgucucgcugguggucuaccguuucgaagauuugaaagugauuaucgaccacuucgagaaauauccgcugauaacacagaaauugggcgauuacaaguuguuuaaacaggcauucagcgucauggagaacaaagaacaucuuaaggagaaugggauuaaggagcucguacgaaucaaagcuaagaugaauuggggucucaaugacgaauugaaaaaagcauuuccagaggugauuagcagggagcgcccccuuaucaauaagaacauuccgaaugggaaauggcuggcuggauucacaucuggugauggcuccuucuucgugcgccuaagaaagucuaauguuaaugcuagaguacgugugcaacugguauucgagaucucacagcacaucagagacaagaaccugaugaauucauugauaacauaccuaggcuguggucacaucuacgagggaaacaaaucugagcgcaguuggcuccaauucagaguagaaaaauucagcgauaucaacgacaagaucauuccgguauuccaggaaaauacucugauuggcaugaaacucgaggacuuugaagauuggugcaagguugccaaauugaucgaagagaagaaacaccugaccgaauccgguuuggaugagauuaagaaaaucaagcugaacaugaacaaaagacgu

SEQ ID NO: 101 - protein, 303 aa, Artificial Sequence
  Engineered variant of I-OnuI LHE
  MOD_RES (1)..(1): Xaa is any amino acid or absent
  MOD_RES (2)..(2): Xaa is any amino acid or absent
  MOD_RES (3)..(3): Xaa is any amino acid or absent
  MOD_RES (4)..(4): Xaa is any amino acid or absent
  MOD_RES (302)..(302): Xaa is any amino acid or absent
  MOD_RES (303)..(303): Xaa is any amino acid or absent
XXXXSRRESINPWTLTGFADAEGSFGLSILNRNRGTARYHTRLSFTIMLHNKDKSILENIQSTWKVGSILNNGDHYVSLVVYRFEDLKVIIDHFEKYPLITQKLGDYKLFKQAFSVMENKEHLKENGIKELVRIKAKMNWGLNDELKKAFPEVISRERPLINKNIPNGKWLAGFTSGDGSFFVRLRKSNVNARVRVQLVFEISQHIRDKNLMNSLITYLGCGHIYEGNKSERSWLQFRVEKFSDINDKIIPVFQENTLIGMKLEDFEDWCKVAKLIEEKKHLTESGLDEIKKIKLNMNKRRXX

SEQ ID NO: 102 - protein, 303 aa, Ophiostoma novo-ulmi
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 103 - protein, 303 aa, Ophiostoma novo-ulmi
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 104 - protein, 303 aa, Ophiostoma novo-ulmi
  MOD_RES (1)..(1): Any amino acid or absent
  MOD_RES (2)..(2): Any amino acid or absent
  MOD_RES (3)..(3): Any amino acid or absent
XXXMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 105 - protein, 303 aa, Ophiostoma novo-ulmi
  MOD_RES (1)..(1): Any amino acid or absent
  MOD_RES (2)..(2): Any amino acid or absent
  MOD_RES (3)..(3): Any amino acid or absent
  MOD_RES (4)..(4): Any amino acid or absent
  MOD_RES (302)..(302): Any amino acid or absent
  MOD_RES (303)..(303): Any amino acid or absent
XXXXSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 106 - protein, 303 aa, Ophiostoma novo-ulmi
  MOD_RES (1)..(1): Any amino acid or absent
  MOD_RES (2)..(2): Any amino acid or absent
  MOD_RES (3)..(3): Any amino acid or absent
  MOD_RES (4)..(4): Any amino acid or absent
  MOD_RES (5)..(5): Any amino acid or absent
  MOD_RES (6)..(6): Any amino acid or absent
  MOD_RES (7)..(7): Any amino acid or absent
  MOD_RES (8)..(8): Any amino acid or absent
  MOD_RES (302)..(302): Any amino acid or absent
  MOD_RES (303)..(303): Any amino acid or absent
XXXXXXXXSINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 107 - DNA, 660 bp, Escherichia coli
atgctgcggatcatagacacggaaacctgtggactccagggtggtatcgtggagatagccagtgtcgatgtaattgatgggaaaatcgtaaacccgatgagccacctcgtccgaccggataggccgataagtccgcaggctatggctatacatagaatcacggaggctatggtagctgacaagccttggatcgaggacgttatcccacactactatgggagcgaatggtatgtagcgcacaacgcatcatttgataggcgcgtgcttcccgaaatgcctggggagtggatttgcacaatgaaactcgctcgccgactgtggcctggtatcaagtatagtaacatggcactttacaagacccgcaaactcaacgtacagacacctccgggtctccaccaccatagggcactctatgattgttacataaccgctgctctccttatagacataatgaatacctccgggtggaccgccgaacaaatggcagacataacgggaagacctagtttgatgacaacctttacatttggaaagtatcggggcaaggcggtgagcgatgtggccgaaagagatcccggttatcttagatggctcttcaataacttggattctatgtcacctgaacttcgccttaccctgaagcattaccttgagaatacc

SEQ ID NO: 108 - RNA, 660 nt, Escherichia coli
augcugcggaucauagacacggaaaccuguggacuccagggugguaucguggagauagccagugucgauguaauugaugggaaaaucguaaacccgaugagccaccucguccgaccggauaggccgauaaguccgcaggcuauggcuauacauagaaucacggaggcuaugguagcugacaagccuuggaucgaggacguuaucccacacuacuaugggagcgaaugguauguagcgcacaacgcaucauuugauaggcgcgugcuucccgaaaugccuggggaguggauuugcacaaugaaacucgcucgccgacuguggccugguaucaaguauaguaacauggcacuuuacaagacccgcaaacucaacguacagacaccuccgggucuccaccaccauagggcacucuaugauuguuacauaaccgcugcucuccuuauagacauaaugaauaccuccggguggaccgccgaacaaauggcagacauaacgggaagaccuaguuugaugacaaccuuuacauuuggaaaguaucggggcaaggcggugagcgauguggccgaaagagaucccgguuaucuuagauggcucuucaauaacuuggauucuaugucaccugaacuucgccuuacccugaagcauuaccuugagaauacc

SEQ ID NO: 109 - protein, 220 aa, Escherichia coli
MLRIIDTETCGLQGGIVEIASVDVIDGKIVNPMSHLVRPDRPISPQAMAIHRITEAMVADKPWIEDVIPHYYGSEWYVAHNASFDRRVLPEMPGEWICTMKLARRLWPGIKYSNMALYKTRKLNVQTPPGLHHHRALYDCYITAALLIDIMNTSGWTAEQMADITGRPSLMTTFTFGKYRGKAVSDVAERDPGYLRWLFNNLDSMSPELRLTLKHYLENT

SEQ ID NO: 110 - DNA, 1425 bp, Escherichia coli
atgaacgatggcaagcagcaaagtacattccttttccacgactacgaaacctttggaacccaccccgctctggatcggccagcacagttcgctgccataagaaccgacaacgagttcaatgtgatcggtgaacccgaggtgttttactgcaaacctgcggacgattatctgcctcaaccgggagccgttctgattacgggaattaccccccaagaagcgcgtgccaagggcgaaaatgaagctgcatttgccgctagaatccatagcctgtttactgttccgaagacgtgtatcttgggatacaacaatgtacgctttgatgacgaagtaactcgtaacgtgttctatcggaacttctatgacccttacgcctggagttggcagcacgacaattctcgatgggatcttctggatgtgatgagagcctgctacgctctcaggccagagggcattaactggcctgagaacgacgatgggcttccgtcatttcgccttgagcatcttaccaaagcgaatggcatcgaacactccaatgcacacgacgctatggctgatgtctacgcgactattgcaatggcgaaattggtcaagacaagacagccgaggttgtttgactacctgtttacgcatcgaaacaaacataagctcatggcactgatagatgtccctcaaatgaagcccctcgttcacgtcagcggtatgttcggagcgtggagaggaaacacgtcctgggttgctcctctggcatggcatccagagaacagaaacgcggtaatcatggtcgatctcgcaggagacattagcccacttctcgaattggattccgacaccttgcgagaacgcctttacacagcgaaaactgatttgggtgataacgctgccgtacctgtcaaattggtacatatcaacaaatgtccggtgctcgcgcaagccaacaccctgagacctgaagacgcggatagactggggataaaccggcagcactgtttggataatctcaagatactcagggagaatccgcaggtgcgcgaaaaagtcgtagccatctttgcggaggcagaaccatttacgccgtctgacaatgtagatgctcagttatacaatggcttctttagtgacgctgaccgagctgcgatgaagattgtgctagagactgaaccgagaaacctctctgcattggacatcacttttgtcgataagcgaatagagaagttgctgttcaactatcgtgcccgaaactttccagggacactcgattacgcagagcaacaaaggtggcttgagcatcgcaggcaggtgtttaccccagagttcttgcagggttacgctgaggagattcaaatgttggcacagcagtatgcagatgataaggagaaagtcgcgttgctgaaggctctgtggcaatacgccgaagagatagtgtga

SEQ ID NO: 111 - RNA, 1425 nt, Escherichia coli
augaacgauggcaagcagcaaaguacauuccuuuuccacgacuacgaaaccuuuggaacccaccccgcucuggaucggccagcacaguucgcugccauaagaaccgacaacgaguucaaugugaucggugaacccgagguguuuuacugcaaaccugcggacgauuaucugccucaaccgggagccguucugauuacgggaauuaccccccaagaagcgcgugccaagggcgaaaaugaagcugcauuugccgcuagaauccauagccuguuuacuguuccgaagacguguaucuugggauacaacaauguacgcuuugaugacgaaguaacucguaacguguucuaucggaacuucuaugacccuuacgccuggaguuggcagcacgacaauucucgaugggaucuucuggaugugaugagagccugcuacgcucucaggccagagggcauuaacuggccugagaacgacgaugggcuuccgucauuucgccuugagcaucuuaccaaagcgaauggcaucgaacacuccaaugcacacgacgcuauggcugaugucuacgcgacuauugcaauggcgaaauuggucaagacaagacagccgagguuguuugacuaccuguuuacgcaucgaaacaaacauaagcucauggcacugauagaugucccucaaaugaagccccucguucacgucagcgguauguucggagcguggagaggaaacacguccuggguugcuccucuggcauggcauccagagaacagaaacgcgguaaucauggucgaucucgcaggagacauuagcccacuucucgaauuggauuccgacaccuugcgagaacgccuuuacacagcgaaaacugauuugggugauaacgcugccguaccugucaaauugguacauaucaacaaauguccggugcucgcgcaagccaacacccugagaccugaagacgcggauagacuggggauaaaccggcagcacuguuuggauaaucucaagauacucagggagaauccgcaggugcgcgaaaaagucguagccaucuuugcggaggcagaaccauuuacgccgucugacaauguagaugcucaguuauacaauggcuucuuuagugacgcugaccgagcugcgaugaagauugugcuagagacugaaccgagaaaccucucugcauuggacaucacuuuugucgauaagcgaauagagaaguugcuguucaacuaucgugcccgaaacuuuccagggacacucgauuacgcagagcaacaaagguggcuugagcaucgcaggcagguguuuaccccagaguucuugcaggguuacgcugaggagauucaaauguuggcacagcaguaugcagaugauaaggagaaagucgcguugcugaaggcucuguggcaauacgccgaagagauaguguga

SEQ ID NO: 112 - protein, 474 aa, Escherichia coli
MNDGKQQSTFLFHDYETFGTHPALDRPAQFAAIRTDNEFNVIGEPEVFYCKPADDYLPQPGAVLITGITPQEARAKGENEAAFAARIHSLFTVPKTCILGYNNVRFDDEVTRNVFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLSALDITFVDKRIEKLLFNYRARNFPGTLDYAEQQRWLEHRRQVFTPEFLQGYAEEIQMLAQQYADDKEKVALLKALWQYAEEIV

SEQ ID NO: 113 - protein, 3 aa, Artificial Sequence
  Exemplary linker sequence
GGG

SEQ ID NO: 114 - protein, 5 aa, Artificial Sequence
  Exemplary linker sequence
DGGGS

SEQ ID NO: 115 - protein, 5 aa, Artificial Sequence
  Exemplary linker sequence
TGEKP

SEQ ID NO: 116 - protein, 4 aa, Artificial Sequence
  Exemplary linker sequence
GGRR

SEQ ID NO: 117 - protein, 5 aa, Artificial Sequence
  Exemplary linker sequence
GGGGS

SEQ ID NO: 118 - protein, 14 aa, Artificial Sequence
  Exemplary linker sequence
EGKSSGSGSESKVD

SEQ ID NO: 119 - protein, 18 aa, Artificial Sequence
  Exemplary linker sequence
KESGSVSSEQLAQFRSLD

SEQ ID NO: 120 - protein, 8 aa, Artificial Sequence
  Exemplary linker sequence
GGRRGGGS

SEQ ID NO: 121 - protein, 9 aa, Artificial Sequence
  Exemplary linker sequence
LRQRDGERP

SEQ ID NO: 122 - protein, 12 aa, Artificial Sequence
  Exemplary linker sequence
LRQKDGGGSERP

SEQ ID NO: 123 - protein, 16 aa, Artificial Sequence
  Exemplary linker sequence
LRQKDGGGSGGGSERP

SEQ ID NO: 124 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 125 - protein, 7 aa, Artificial Sequence
  Cleavage sequence by TEV protease
ENLYFQG

SEQ ID NO: 126 - protein, 7 aa, Artificial Sequence
  Cleavage sequence by TEV protease
ENLYFQS

SEQ ID NO: 127 - protein, 22 aa, Artificial Sequence
  Self-cleaving polypeptide comprising 2A site
GSGATNFSLLKQAGDVEENPGP

SEQ ID NO: 128 - protein, 19 aa, Artificial Sequence
  Self-cleaving polypeptide comprising 2A site
ATNFSLLKQAGDVEENPGP

SEQ ID NO: 129 - protein, 14 aa, Artificial Sequence
  Self-cleaving polypeptide comprising 2A site
LLKQAGDVEENPGP

SEQ ID NO: 130 - protein, 21 aa, Artificial Sequence
  Self-cleaving polypeptide comprising 2A site
GSGEGRGSLLTCGDVEENPGP

SEQ ID NO: 131 - protein, 18 aa, Artificial Sequence
  Self-cleaving polypeptide comprising 2A site
EGRGSLLTCGDVEENPGP

SEQ ID NO: 132 - protein, 13 aa, Artificial Sequence
  Self-cleaving polypeptide comprising 2A site
LLTCGDVEENPGP

SEQ ID NO: 133 - protein, 23 aa, Artificial Sequence
  Self-cleaving polypeptide comprising 2A site
GSGQCTNYALLKLAGDVESNPGP

SEQ ID NO: 134 - protein, 20 aa, Artificial Sequence
  Self-cleaving polypeptide comprising 2A site
QCTNYALLKLAGDVESNPGP

SEQ ID NO: 135 - protein, 14 aa, Artificial Sequence
  Self-cleaving polypeptide comprising 2A site
LLKLAGDVESNPGP

SEQ ID NO: 136 - protein, 25 aa, Artificial Sequence
  Self-cleaving polypeptide comprising 2A site
GSGVKQTLNFDLLKLAGDVESNPGP

SEQ ID NO: 137 - protein, 22 aa, Artificial Sequence
  Self-cleaving polypeptide comprising 2A site
VKQTLNFDLLKLAGDVESNPGP

SEQ ID NO: 138 - protein, 14 aa, Artificial Sequence
  Self-cleaving polypeptide comprising 2A site
LLKLAGDVESNPGP

SEQ ID NO: 139 - protein, 19 aa, Artificial Sequence
  Self-cleaving polypeptide comprising 2A site
LLNFDLLKLAGDVESNPGP

SEQ ID NO: 140 - protein, 19 aa, Artificial Sequence
  Self-cleaving polypeptide comprising 2A site
TLNFDLLKLAGDVESNPGP

SEQ ID NO: 141 - protein, 14 aa, Artificial Sequence
  Self-cleaving polypeptide comprising 2A site
LLKLAGDVESNPGP

SEQ ID NO: 142 - protein, 17 aa, Artificial Sequence
  Self-cleaving polypeptide comprising 2A site
NFDLLKLAGDVESNPGP

SEQ ID NO: 143 - protein, 20 aa, Artificial Sequence
  Self-cleaving polypeptide comprising 2A site
QLLNFDLLKLAGDVESNPGP

SEQ ID NO: 144 - protein, 24 aa, Artificial Sequence
  Self-cleaving polypeptide comprising 2A site
APVKQTLNFDLLKLAGDVESNPGP

SEQ ID NO: 145 - protein, 40 aa, Artificial Sequence
  Self-cleaving polypeptide comprising 2A site
VTELLYRMKRAETYCPRPLLAIHPTEARHKQKIVAPVKQT

SEQ ID NO: 146 - protein, 18 aa, Artificial Sequence
  Self-cleaving polypeptide comprising 2A site
LNFDLLKLAGDVESNPGP

SEQ ID NO: 147 - protein, 40 aa, Artificial Sequence
  Self-cleaving polypeptide comprising 2A site
LLAIHPTEARHKQKIVAPVKQTLNFDLLKLAGDVESNPGP

SEQ ID NO: 148 - protein, 33 aa, Artificial Sequence
  Self-cleaving polypeptide comprising 2A site
EARHKQKIVAPVKQTLNFDLLKLAGDVESNPGP

SEQ ID NO: 149 - DNA, 10 bp, Unknown
  Kozak sequence
gccrccatgg

SEQ ID NO: 150 - protein, 10 aa, Artificial Sequence
  Exemplary linker sequence
GGGGSGGGGS

SEQ ID NO: 151 - protein, 15 aa, Artificial Sequence
  Exemplary linker sequence
GGGGSGGGGSGGGGS

SEQ ID NO: 152 - protein, 20 aa, Artificial Sequence
  Exemplary linker sequence
GGGGSGGGGSGGGGSGGGGS

SEQ ID NO: 153 - protein, 25 aa, Artificial Sequence
  Exemplary linker sequence
GGGGSGGGGSGGGGSGGGGSGGGGS

SEQ ID NO: 154 - DNA, 41 bp, Artificial Sequence
  Made in Lab - PD-1 megaTAL polynucelotide section
ctggtggggctgctccaggcatgcagatcccacaggcgccc

SEQ ID NO: 155 - DNA, 38 bp, Artificial Sequence
  Made in Lab - PD-1 megaTAL polynucelotide section with deletion
ctggtggggctgctccaggcatgcagccacaggcgccc

SEQ ID NO: 156 - DNA, 37 bp, Artificial Sequence
  Made in Lab - PD-1 megaTAL polynucelotide section with deletion
ctggtggggctgctccaggcatgcagcacaggcgccc

SEQ ID NO: 157 - DNA, 36 bp, Artificial Sequence
  Made in Lab - PD-1 megaTAL polynucelotide section with deletion
ctggtggggctgctccaggcatgcacacaggcgccc

SEQ ID NO: 158 - DNA, 26 bp, Artificial Sequence
  Made in Lab - PD-1 megaTAL polynucelotide section with deletion
ctggtggggctgctccacaggcgccc

SEQ ID NO: 159 - protein, 7 aa, Artificial Sequence
  Made in Lab - PD-1 megaTAL polypeptide section
MQIPQAP

SEQ ID NO: 160 - protein, 6 aa, Artificial Sequence
  Made in Lab - PD-1 megaTAL polypeptide section with deletion
MQPQAP

SEQ ID NO: 161 - protein, 6 aa, Artificial Sequence
  Made in Lab - PD-1 megaTAL polypeptide section with deletion
MQHRRP

SEQ ID NO: 162 - protein, 6 aa, Artificial Sequence
  Made in Lab - PD-1 megaTAL polypeptide section with deletion
MHTGAL

SEQ ID NO: 163 - DNA, 29 bp, Homo sapiens
gtggggtatgtgtgtgtccggggctgccg

SEQ ID NO: 164 - DNA, 21 bp, Homo sapiens
aacaaaagcggctctgagtct

(19) United States
(12) Patent Application Publication
(10) Pub. No.: US 2024/0336904 A1
(43) Pub. Date: Oct. 10, 2024

(54) COMPOSITIONS AND METHODS FOR SITE-DIRECTED MUTAGENESIS

(71) Applicant: 2seventy bio, Inc., Cambridge, MA (US)

(72) Inventors: JORDAN JARJOUR, SEATTLE, WA (US); ANNE-RACHEL KROSTAG, LAKE FOREST PARK, WA (US)

(73) Assignee: 2seventy bio, Inc., Cambridge, MA (US)

(21) Appl. No.: 18/258,021

(22) PCT Filed: Dec. 16, 2021
(86) PCT No.: PCT/US2021/063771

§ 371 Date: Jun. 16, 2023

Related U.S. Application Data
(60) Provisional application No. 63/128,391, filed on Dec. 21, 2020.

(51) Int. Cl. C12N 9/22; A61K 35/17; C12N 5/0783; C12N 15/10

(52) U.S. Cl. CPC C12N 9/22; A61K 35/17; C12N 5/0636; C12N 15/102; C07K 2319/81; C12N 2510/00; C12Y 301/11001; C12Y 301/11002

(57) ABSTRACT

The present disclosure provides improved genome editing compositions and methods for editing a double-strand DNA target site. The disclosure further provides genome edited cells produced by the compositions and methods described.

### CROSS REFERENCE TO RELATED APPLICATIONS

This application claims the benefit under 35 U.S.C. § 119 (e) of U.S. Provisional Application No. 63/128,391, filed Dec. 21, 2020, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLUE-132PC_ST25.txt. The text file is 541 KB, created on Dec. 15, 2021, and is being submitted electronically via EFS-Web.

### BACKGROUND

### Technical Field

The present disclosure relates to improved genome editing compositions. More particularly, the disclosure relates to fusion polypeptides comprising a DNA-binding domain and a homing endonuclease variant linked to an exonuclease, compositions, and methods of using the same for site-directed mutagenesis of dsDNA within a cell.

### Description of the Related Art

The relatively recent surge in genome editing technologies has opened up the possibility of directly targeting and modifying genomic sequences in almost all eukaryotic cells and mammals. Such technologies include, but are not limited to, transcription activator-like effector nucleases (TALENs), zinc-finger nucleases (ZFNs), clustered regularly interspaced short palindromic repeat (CRISPR)-Cas-associated nucleases, and homing endonucleases (HEs). Common to all of these editing techniques is that they create a breakpoint in the target nucleotide sequence, while the natural cellular repair mechanisms are left to re-ligate the nucleotide sequence either by non-homologous end-joining (NHEJ) or homology-directed repair (HDR). However, the repair is not always perfect. Thus, the end product is a nucleotide sequence comprising any one of a variety of genetic lesions.

The most frequently observed genetic lesions resulting from the application of gene editing nucleases are insertions and deletions (customarily referred to as ‘indels’). Indels arise when double-stranded DNA breaks (DSB) are processed and re-sealed by NHEJ DNA repair machinery. NHEJ indels typically predominate gene editing events in the absence of a delivered excess of homologous DNA sequence that can divert DSBs toward various homologous recombination outcomes.

The specific properties of each genetic lesion can lead to different phenotypic outcomes. For example, any given genetic lesion may result in a wide array of phenotypic outcomes spanning from a total knockout of a gene, to a gain or loss of function, to no phenotype affect at all. Accordingly, there is a growing need to further understand the mechanisms that lead to different mutations upon genetic editing, and to develop compositions and methods to effectuate therapeutically meaningful genetic edits.

### BRIEF SUMMARY

The present disclosure generally relates, in part, to fusion polypeptides comprising a DNA-binding domain, a homing endonuclease variant that cleaves a target site in the human gene, a linker domain, and an exonuclease, and methods of using the same.

In one aspect, a fusion polypeptide is provided comprising, a DNA-binding domain and a homing endonuclease (HE) variant that binds and cleaves a selected double strand DNA (dsDNA) target site in a cell; a linker domain; and an exonuclease or biologically active fragment thereof.

In particular embodiments, the exonuclease is Trex2, ExoI, or ExoX, or biologically active fragment thereof. In some embodiments, the exonuclease is ExoX, or biologically active fragment thereof. In some embodiments, the exonuclease is ExoI, or biologically active fragment thereof. In some embodiments, the exonuclease is Trex2, or biologically active fragment thereof.

In various embodiments, the homing endonuclease is an engineered homing endonuclease.

In various embodiments, the selected dsDNA target site is a non-native homing endonuclease target site.

In various embodiments, the DNA-binding domain binds a dsDNA target site upstream of the endonuclease dsDNA target site. In some embodiments, the DNA-binding domain comprises a TALE DNA-binding domain. In some embodiments, the TALE DNA domain comprising about 9.5 TALE repeat units to about 15.5 TALE repeat units. In some embodiments, the TALE DNA domain comprising 11.5 TALE repeat units or 12.5 TALE repeat units. In some embodiments, the DNA-binding domain comprises a zinc finger DNA-binding domain. In some embodiments, the zinc finger DNA-binding domain comprises 2, 3, 4, 5, 6, 7, or 8 zinc finger motifs.

In various embodiments, the linker domain is a peptide linker. In some embodiments, the peptide linker is a self-cleaving peptide linker. In some embodiments, the peptide linker comprises about 4 to about 30 amino acids. In some embodiments, the peptide linker comprises about 10 to about 16 amino acids. In some embodiments, the peptide linker comprises about 12 amino acids. In some embodiments, the peptide linker is a (GGGGS) 1-4 linker (SEQ ID NOs: 117, 150-152). In some embodiments, the peptide linker comprises a (GGGGS) 2 linker (SEQ ID NO: 150).

In various embodiments, the HE variant is an LAGLIDADG homing endonuclease (LHE) variant. In some embodiments, the HE variant lacks the 1, 2, 3, 4, 5, 6, 7, or 8 N-terminal amino acids compared to a corresponding wild type HE. In some embodiments, the HE variant lacks the 4 N-terminal amino acids compared to a corresponding wild type HE. In some embodiments, the HE variant lacks the 8 N-terminal amino acids compared to a corresponding wild type HE. In some embodiments, the HE variant lacks the 1, 2, 3, 4, or 5 C-terminal amino acids compared to a corresponding wild type HE. In some embodiments, the HE variant lacks the C-terminal amino acid compared to a corresponding wild type HE. In some embodiments, the HE variant lacks the 2 C-terminal amino acids compared to a corresponding wild type HE.

In particular embodiments, the HE variant is a variant of an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-SceI, I-ScuMI, I-SmaMI, I-SscMI, and I-Vdi141I. In some embodiments, the HE variant is a variant of an LHE selected from the group consisting of: I-CpaMI, I-HjeMI, I-OnuI, I-PanMI, and I-SmaMI. In a preferred embodiments, the HE variant is an I-OnuI LHE variant.

In various embodiments, the HE target site is within a immune system checkpoint gene, globin gene, gene that encodes a polypeptide that contributes to repression of γ-globin gene expression and HbF, or immunosuppressive signaling gene. In some embodiments, the HE target site is within a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), TCRβ, IL10Rα, IL10Rβ, TGFBR1, TGFBR2, CBL-B, PCSK9, AHR, BTK, α-globin, β-globin, γ-globin, BCL11A, KLF1, SOX6, GATA1, LSD1, alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), TEM5, TEM8, trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1) gene. In some embodiments, the HE target site is within a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), IL10Rα, TGFBR2, CBL-B, PCSK9, AHR, BTK, α-globin, β-globin, γ-globin, and BCL11A gene. In some embodiments, the HE target site is within a TRAC (TCRα) gene, a CBL-B gene, or a PDCD1 (PD-1) gene. In particular embodiments, the TCRα gene target site comprises the amino acid sequence set forth in SEQ ID NO: 1. In particular embodiments, the CBL-B gene target site comprises the amino acid sequence set forth in SEQ ID NO: 2. In particular embodiments, the PD-1 gene target site comprises the amino acid sequence set forth in SEQ ID NO: 3.

In various embodiments, the DNA-binding domain comprises a TALE DNA-binding domain having a target site as set forth in SEQ ID NO: 4.

In various embodiments, the ExoX, or biologically active fragment thereof, comprises an amino acid sequence having at least 85%, 90%, 95%, 96%, 97%, 98%, or 99% identity to an amino acid sequence as set forth in SEQ ID NO: 109. In particular embodiments, the ExoX, or biologically active fragment thereof, comprises an amino acid an amino acid sequence as set forth in SEQ ID NO: 109.

In various embodiments, the fusion polypeptide comprises an amino acid sequence having at least 85%, 90%, 95%, 96%, 97%, 98%, or 99% identity to an amino acid sequence as set forth in any one of SEQ ID NOs: 46, 64, 73, and 82. In particular embodiments, the fusion polypeptide comprises an amino acid sequence as set forth in any one of SEQ ID NOs: 46, 64, 73, and 82.

In various embodiments, the ExoI, or biologically active fragment thereof, comprises an amino acid an amino acid sequence having at least 85%, 90%, 95%, 96%, 97%, 98%, or 99% identity to an amino acid sequence as set forth in SEQ ID NO: 112. In particular embodiments, the ExoI, or biologically active fragment thereof, comprises an amino acid an amino acid sequence as set forth in SEQ ID NO: 112.

In various embodiments, the fusion polypeptide comprises an amino acid sequence having at least 85%, 90%, 95%, 96%, 97%, 98%, or 99% identity to an amino acid sequence as set forth SEQ ID NOs: 43. In particular embodiments, the fusion polypeptide comprises an amino acid sequence as set forth SEQ ID NOs: 43.

In various embodiments, a polynucleotide encoding the fusion polypeptide contemplated herein is provided. In some embodiments, the polynucleotide comprises a nucleotide sequence having at least 85%, 90%, 95%, 96%, 97%, 98%, or 99% identity to a polynucleotide sequence as set forth in any one of SEQ ID NOs: 44, 62, 71, and 80. In particular embodiments, the polynucleotide comprises a polynucleotide sequence as set forth in any one of SEQ ID NOs: 44, 62, 71, and 80.

In various embodiments, an mRNA encoding the fusion polypeptide contemplated herein is provided. In some embodiments, the mRNA comprises an RNA sequence having at least 85%, 90%, 95%, 96%, 97%, 98%, or 99% identity to an RNA sequence as set forth in any one of SEQ ID NOs: 45, 63, 72, and 81. In particular embodiments, the mRNA comprises an RNA sequence as set forth in any one of SEQ ID NOs: 45, 63, 72, and 81. In some embodiments, the mRNA comprises an RNA sequence having at least 85%, 90%, 95%, 96%, 97%, 98%, or 99% identity to an RNA sequence as set forth in SEQ ID NOs: 42. In particular embodiments, the mRNA comprises an RNA sequence as set forth in any one of SEQ ID NOs: 42.

In various embodiments, a vector encoding the fusion polypeptide contemplated herein is provided. In some embodiments, the vector comprises a polynucleotide encoding the fusion polypeptide contemplated herein.

In various embodiments, a cell comprising the fusion polypeptide encoding the fusion polypeptide contemplated herein is provided. In some embodiments, the cell comprises a polynucleotide encoding the fusion polypeptide contemplated herein. In some embodiments, comprises a mRNA encoding the fusion polypeptide contemplated herein. In some embodiments, the cell comprises a vector contemplated herein. In some embodiments, the cell comprises one or more genome modifications.

In various embodiments, the cell is a hematopoietic cell. In some embodiments, the cell is a hematopoietic stem or progenitor cell. In some embodiments, the cell is a CD34+ cell. In some embodiments, the cell is a CD133+ cell.

In various embodiments, the cell is an immune effector cell. In some embodiments, the immune effector cell is a cytotoxic T lymphocytes (CTLs), a tumor infiltrating lymphocytes (TILs), or a helper T cell. In some embodiments, the immune effector cell is a T cell. In some embodiments, the immune effector cell is an αβ T cell, a γδ T cell, a natural killer (NK) cell, or a natural killer T (NKT) cell.

In various embodiments, a population of cells contemplated herein is provided.

In various embodiments, a composition comprising the fusion polypeptide contemplated herein is provided. In some embodiments, a composition comprising the polynucleotides contemplated herein is provided. In some embodiments, a composition comprising the mRNA contemplated herein is provided. In some embodiments, a composition comprising the vector contemplated herein is provided. In some embodiments, a composition comprising the cell contemplated herein is provided. In some embodiments, a composition comprising the population of cells contemplated herein is provided. In particular embodiments, the composition comprises a pharmaceutically acceptable carrier.

In another aspect, a method of site-directed mutagenesis is provided comprising: (a) selecting a double-stranded DNA (dsDNA) target site, and (b) introducing into the cell a fusion polypeptide, polynucleotide, mRNA, or vector contemplated herein; wherein the fusion peptide generates directionally biased deletions having a deletion center location near the selected dsDNA target cut site in the cell.

In various embodiments, greater than 50%, greater than 51%, greater than 52%, greater than 53%, greater than 54%, greater than 55%, greater than 56%, greater than 57%, greater than 58%, greater than 59%, greater than 60%, greater than 65%, greater than 70%, greater than 75%, or greater than 80% of the directionally biased deletions have a deletion center location on one side of the HE target site center location.

In various embodiments, the deletion center location is on the same side as the DNA-binding domain target site relative to the HE target site center location. In a particular embodiments, the deletion center location is 5′ to the HE target site center location.

In various embodiments, at least 50%, at least 51%, at least 52%, at least 53%, at least 54%, at least 55%, at least 56%, at least 57%, at least 58%, at least 59%, at least 60%, at least 65%, at least 70%, at least 75%, or at least 80% of deletions have a deletion center greater than 4 nucleotides away from the HE target site center location.

In various embodiments, at least 10%, at least 11%, at least 12%, at least 13%, at least 14%, at least 15%, at least 16%, at least 17%, at least 18%, at least 19%, at least 20%, at least 25%, at least 30%, or at least 35% of deletions have a deletion center greater than 8 nucleotides away from the HE target site center location.

In various embodiments, at least 50%, at least 51%, at least 52%, at least 53%, at least 54%, at least 55%, at least 56%, at least 57%, at least 58%, at least 50%, at least 59%, at least 60%, at least 65%, at least 70%, at least 75%, or at least 80% of deletions are 6 bps in length or greater.

In various embodiments, at least 30%, at least 31%, at least 32%, at least 33%, at least 34%, at least 35%, at least 36%, at least 37%, at least 38%, at least 39%, at least 40%, at least 45%, at least 50%, at least 55%, or at least 60% of deletions are 12 bps in length or greater.

In various embodiments, the directionally biased deletions comprise a length of about 10, about 11, about 12, about 13, about 14, about 15, about 16, about 17, about 18, about 19, or about 20 nucleotides.

In various embodiments, the deletion extends into the DNA-binding domain target site. In some embodiments, the deletion center location is within the DNA-binding domain target site.

In various embodiments, the method further comprises introducing into the cell an end-processing enzyme, or biologically active fragment thereof. In some embodiments, the end-processing enzyme, or biologically active fragment thereof, is selected from the group consisting of: Trex2, Trex1, Trex1 without transmembrane domain, Apollo, Artemis, DNA2, ExoI, ExoT, ExoIII, ExoX, Fen1, Fen1, MreII, Rad2, Rad9, TdT (terminal deoxynucleotidyl transferase), PNKP, RecE, RecJ, RecQ, Lambda exonuclease, Sox, Vaccinia DNA polymerase, exonuclease I, exonuclease III, exonuclease VII, NDK1, NDK5, NDK7, NDK8, WRN, T7-exonuclease Gene 6, avian myeloblastosis virus integration protein (IN), Bloom, Antartic Phophatase, Alkaline Phosphatase, Poly nucleotide Kinase (PNK), ApeI, Mung Bean nuclease, Hex1, TTRAP (TDP2), Sgs1, Sae2, CUP, Pol mu, Pol lambda, MUS81, EME1, EME2, SLX1, SLX4 and UL-12. In some embodiments, the end processing enzyme is an exonuclease, or biologically active fragment thereof. In particular embodiments, the exonuclease is Trex2, or biologically active fragment thereof.

In various embodiments, the method is an in vitro method. In various embodiments, the method is an ex vivo method. In various embodiments, the method is an in vivo method.

In another aspect, a method of treating, preventing, or ameliorating at least one symptom of a disease, or condition associated therewith is provided, comprising harvesting a population of cells from a subject; editing the population of cells according to a method contemplated herein, and administering the edited population of cells to the subject.

In another aspect, use of the cells contemplated herein for treating, preventing, or ameliorating at least one symptom of a disease or condition associated therewith is provided.

In another aspect, use of the population contemplated herein for treating, preventing, or ameliorating at least one symptom of a disease or condition associated therewith is provided.

In another aspect, use of the compositions contemplated herein for treating, preventing, or ameliorating at least one symptom of a disease or condition associated therewith is provided.

In various embodiments, the disease or condition is an immune disorder or cancer.

### BRIEF DESCRIPTION OF SEVERAL VIEWS OF THE DRAWINGS

FIG. 1A shows a cartoon of a TALE DNA-binding domain linked to a homing endonuclease reprogramed to target a TCRα nucleotide sequence (TCRα megaTAL).

FIG. 1B shows the indel activity by next-gen sequencing (NGS) for a TCRα megaTAL. The NGS data reads containing indels characteristic of gene editing events are tabulated according to their length.

FIG. 2 shows indel position distributions (“fingerprints”) as assessed by NGS for both a low activity and high activity TCRα megaTALs, in two different donors. The NGS data are tabulated according to both their length and their longitudinal location relative to the megaTAL caused breakpoint center. Insertions are excluded from this analysis.

FIG. 3A-3C show indel activity by NGS for a TCRα megaTAL (FIG. 3A), TCRα megaTAL-Trex2 fusion (FIG. 3B), and a TCRα megaTAL+Trex2 co-expression (FIG. 3C). The NGS data reads containing indels characteristic of gene editing events are tabulated according to their length.

FIG. 4 shows indel position distributions (“fingerprints”) as assessed by NGS for a TCRα megaTAL, TCRα megaTAL-Trex2 fusion, and TCRα megaTAL+Trex2 co-expression. The NGS data are tabulated according to both their length and their longitudinal location relative to the megaTAL caused breakpoint center. Insertions are excluded from this analysis.

FIG. 5 shows editing efficiency of TCRα megaTALs fused to various Trex2 homologs as assessed by staining for the CD3 components that combine with the TCR alpha and beta chains on the cell surface, followed by flowcytometry analysis.

FIG. 6 shows indel activity by NGS for TCRα megaTALs fused to Trex2 homologs. The NGS data reads containing indels characteristic of gene editing events are tabulated according to their length.

FIG. 7 shows indel position distributions (“fingerprints”) as assessed by NGS for a TCRα megaTALs fused to Trex2 platypus, opossum, human, and mouse homologues. The NGS data are tabulated according to both their length and their longitudinal location relative to the megaTAL caused breakpoint center. Insertions are excluded from this analysis.

FIG. 8 shows editing efficiency of TCRα megaTALs fused to exonucleases RAD1, RAD9A, ExoI, ExoX, T5FEN, lanbdaExo, and RecJ, with or without co-expression of mouse Trex2. Trex2 homologs as assessed by staining for the CD3 expression followed by flowcytometry analysis.

FIG. 9 shows indel position distributions (“fingerprints”) as assessed by NGS for a TCRα megaTALs alone, and TCRα megaTALs fused to exonucleases ExoI or ExoX, with or without co-expression of Trex2. The NGS data are tabulated according to both their length and their longitudinal location relative to the megaTAL caused breakpoint center. Insertions are excluded from this analysis.

FIG. 10 shows indel position distributions (“fingerprints”) as assessed by NGS for high-activity CBL-B megaTALs alone or fused to Trex2 or ExoX. The NGS data are tabulated according to both their length and their longitudinal location relative to the megaTAL caused breakpoint center. Insertions are excluded from this analysis.

FIG. 11 shows indel position distributions (“fingerprints”) as assessed by NGS for low-activity CBL-B megaTALs alone or fused to Trex2 or ExoX. The NGS data are tabulated according to both their length and their longitudinal location relative to the megaTAL caused breakpoint center. Insertions are excluded from this analysis.

FIG. 12 shows indel position distributions (“fingerprints”) as assessed by NGS for PD-1 megaTALs alone or fused to Trex2 or ExoX. The NGS data are tabulated according to both their length and their longitudinal location relative to the megaTAL caused breakpoint center. Insertions are excluded from this analysis.

FIG. 13 shows the PDCD1 gene, the 22 base pair PD-1 megaTAL target site, and the 13 base TALE array binding site (SEQ ID NOs 154 and 159), as well as 4 representative deletion species (SEQ ID NOs: 155-158 and 160-162) from the experiment shown in FIG. 12.

FIG. 14 shows indel position distributions (“fingerprints”) as assessed by NGS for PD-1 megaTALs alone or fused to Trex2 or ExoX. The deletion species are coded to indicate their resulting reading frame category. The NGS data are tabulated according to both their length and their longitudinal location relative to the megaTAL caused breakpoint center. Insertions are excluded from this analysis.

FIG. 15 shows stacked histograms quantifying the normalized fraction of each of the four deletion species categories shown in FIG. 14.

FIG. 16 shows flow cytometry analysis of activated primary human T cells electroporated with poly-adenylated mRNA encoding either or both a cyan fluorescent protein (CFP) to track transfection efficiency, and wild-type or mock-edited PD-1 alleles in each of the three possible reading frames.

FIG. 17 shows indel position distributions (“fingerprints”) as assessed by NGS for a low editing TCRα megaTAL, high editing TCRα megaTAL (TCRα 2.2), as well as direct fusions of each to Trex2 or ExoX. The NGS data are tabulated according to both their length and their longitudinal location relative to the megaTAL caused breakpoint center. Insertions are excluded from this analysis.

FIG. 18 shows indel position distributions (“fingerprints”) at the known KAT2B off-target site for the low-editing TCRα megaTAL, with and without direct fusion to Trex2 or ExoX. The NGS data are tabulated according to both their length and their longitudinal location relative to the megaTAL caused breakpoint center. Insertions are excluded from this analysis.

FIG. 19 shows indel position distributions (“fingerprints”) at the known AC016700.3 off-target site (SEQ ID NOs: 163 and 164) for the high-editing TCRα megaTAL (TCRα 2.2), with and without direct fusion to ExoX. The NGS data are tabulated according to both their length and their longitudinal location relative to the megaTAL caused breakpoint center. Insertions are excluded from this analysis.

### BRIEF DESCRIPTION OF THE SEQUENCE IDENTIFIERS

SEQ ID NOs: 1-3 are illustrative homing endonuclease target sites.

SEQ ID NO: 4 is PD-1 TALE array target site.

SEQ ID NO: 5-7 are low activity TCRα megaTAL DNA, RNA, and protein sequences.

SEQ ID NO: 8-10 are high activity TCRα megaTAL DNA, RNA, and protein sequences.

SEQ ID NO: 11-13 are low activity TCRα megaTAL-Trex2 DNA, RNA, and protein sequences.

SEQ ID NOs: 14-16 are Trex2 exonuclease DNA, RNA, and protein sequences.

SEQ ID NOs: 17-55 are DNA, RNA and protein sequences for low activity TCRα megaTALs fused to Trex2 homologs.

SEQ ID NOs: 56-64 are DNA, RNA, and protein sequences for high activity CBL-B megaTALs with and without fusion to Trex2 or ExoX.

SEQ ID NO: 65-73 are DNA, RNA, and protein sequences for low activity CBL-B megaTALs with and without fusion to Trex2 or ExoX.

SEQ ID NO: 74-82 are DNA, RNA, and protein sequences for PD-1 megaTALs with and without fusion to Trex2 or ExoX.

SEQ ID NO: 83 is an mRNA sequence encoding PD-1.

SEQ ID NO: 84 is an mRNA sequence encoding a mock-edited PD-1 open reading frame (ORF) comprising a 1 bp deletion at codon 3.

SEQ ID NO: 85 is an mRNA sequence encoding a mock-edited PD-1 open reading frame (ORF) comprising a 2 bp deletion at codon 3.

SEQ ID NO: 86 is an mRNA sequence encoding a mock-edited PD-1 open reading frame (ORF) comprising a 3 bp deletion at codon 3.

SEQ ID NOs: 87-101 are DNA, RNA, and protein sequences for TCRα, CBL-B, and PD-1 homing endonucleases.

SEQ ID NOs: 102-106 are wild type I-OnuI endonucleases and portions thereof.

SEQ ID NOs: 107-109 are ExoX exonuclease DNA, RNA, and protein sequences.

SEQ ID NOs: 110-112 are ExoI exonuclease DNA, RNA, and protein sequences.

SEQ ID NO: 113-123 sets forth the amino acid sequence of various linkers.

SEQ ID NOs: 124-148 sets forth the amino acid sequence of protease cleavage sites and self-cleaving polypeptide cleavage sites.

In the foregoing sequences, X, if present, refers to any amino acid or the absence of an amino acid.

### DETAILED DESCRIPTION

### A. Overview

The present disclosure generally relates to, in part, improved genome editing compositions and methods of use thereof. Without wishing to be bound by any particular theory, the genome editing compositions contemplated herein are used to 1) increase the size of deletions induced by a genetic edit to a specific target size and 2) bias the deletion center location. In particular embodiments, the deletion center locations are predominantly on the same side as the DNA-binding domain target site relative to the original breakpoint center or endonuclease target site center. In particular embodiments, the deletion center locations are biased to the 5′ side of the original breakpoint center or endonuclease target site center. It is further contemplated that by controlling the size and location of genetic lesions at or proximal to a breakpoint, one can more precisely control the phenotypic outcome of the genomic edit, for example, disruption of regulatory DNA sequences and/or target gene expression.

The most frequently observed genetic lesions resulting from the application of gene editing nucleases are NHEJ indels. Little is known regarding how the NHEJ machinery arrives at a given indel that is re-sealed and resistant to further DNA cleavage, thus becoming enshrined as a permanent genotype within a pool of gene edited alleles. However, it is known that indel events are almost exclusively observed at, and directly proximal to, the DNA sequence where nuclease generated breakpoint(s) occur. Previous studies with various nuclease platforms (ZFN, TALEN, and CRISPR) have suggested that the gene edited alleles show some measure of consistency with respect to the qualitative properties of the observed indels. This implies that there are determinative biophysical and/or biochemical processes that govern gene editing outcomes. However, while there are some subtle differences in the observed spectra of edited alleles arising from different gene editing nucleases across different DNA target sites, the capacity to influence indel properties has remained elusive.

The qualitative properties that define a given indel are: (i) its length, in number of bases inserted or deleted; (ii) its longitudinal position along the chromosome, usually stated relative to the nuclease target site or breakpoint; and (iii) for insertions, the inserted sequence length and composition. Deletions are the most prominent outcomes, typically comprising 90-95% of the observed events. Their most commonly reported size characteristics tend to small (i.e. 1-20 base pairs in length, with the frequency biased toward the low end of that range) and their positional distributions have been found to be evenly distributed, covering the DNA breakpoint and emanating outward in either direction without significant bias. Exceptions to these properties are frequently hypothesized to be driven by microhomologies (small duplicated tracts of approximately 3-6 base pairs in length) positioned on either side of the DNA breakpoint. Little has been reported regarding the properties of the insertions that occur far less frequently during the application of genome editing tools. Additionally, the genotypic characteristics that relate each indel species to a phenotype (for example, how it impacts an open reading frame or whether it disrupts a transcription factor binding motif) are potentially vast and idiosyncratic to each given application.

Unique fusion polypeptides having distinct editing patterns (e.g., deletions), which are useful for precisely controlling the phenotypic outcome of the genomic edit, are contemplated herein. In various embodiments, the fusion polypeptides comprise a DNA-binding domain, a homing endonuclease variant, and an end-processing enzyme (e.g., exonuclease). In certain embodiments, the exonuclease is ExoX, ExoI, or a biologically active fragment thereof. In particular embodiments, the fusion polypeptides induce elongated genetic deletions having directionally biased deletion centers on the same side as the DNA-binding domain target site, or 5′, to that of the original edit/breakpoint. In particular embodiments, the fusion polypeptides generate deletion center locations encompassed by the DNA-binding domain target site. In particular embodiments, the fusion polypeptides generate deletions that extend into (or are encompassed by) the DNA-binding domain target site.

In particular embodiments, the fusion polypeptide comprises a DNA-binding domain and a homing endonuclease (HE) variant that binds and cleaves a selected double strand DNA (dsDNA) target site in a cell, a polypeptide linker and an exonuclease (e.g., ExoX or ExoI), or biologically active fragment thereof.

In various embodiments, vectors, polynucleotides, mRNA, or cDNA encoding the fusion polypeptides are contemplated. In various embodiments, genome edited cells are contemplated. In various embodiments, compositions comprising the fusion polypeptides, vectors, polynucleotides, mRNA, cDNA or cells are contemplated.

In various embodiments, methods of genome editing, site-directed mutagenesis, increasing the length of site-directed mutagenic deletions, biasing the location of deletions, and treating subjects in need thereof are contemplated.

Accordingly, the compositions and methods contemplated herein represent a significant improvement compared to existing gene editing strategies because it allows for strategic control and selection of desired mutagenic outcomes.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism. In one embodiment, cellular genomes are engineered, edited, or modified in vivo.

By “enhance” or “promote” or “increase” or “expand” or “potentiate” refers generally to the ability of a fusion polypeptide, nuclease variant, genome editing composition, or genome edited cell contemplated herein to produce, elicit, or cause a greater response (i.e., physiological response) compared to the response caused by either vehicle or control. A measurable response may include an increase in editing events (e.g., indels), deletions, insertions, deletion length, and/or target gene expression, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or control.

By “decrease” or “lower” or “lessen” or “reduce” or “abate” or “ablate” or “inhibit” or “dampen” refers generally to the ability of fusion polypeptide, nuclease variant, genome editing composition, or genome edited cell contemplated herein to produce, elicit, or cause a lesser response (i.e., physiological response) compared to the response caused by either vehicle or control. A measurable response may include a decrease in editing events (e.g., indels), deletions, insertions, deletion length, target gene expression, and/or one or more symptoms associated with a disease. A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, or control.

By “maintain,” or “preserve,” or “maintenance,” or “no change,” or “no substantial change,” or “no substantial decrease” refers generally to the ability of a fusion polypeptide, nuclease variant, genome editing composition, or genome edited cell contemplated herein to produce, elicit, or cause a substantially similar or comparable physiological response (i.e., downstream effects) in as compared to the response caused by either vehicle or control. A comparable response is one that is not significantly different or measurable different from the reference response.

The terms “specific binding affinity” or “specifically binds” or “specifically bound” or “specific binding” or “specifically targets” as used herein, describe binding of one molecule to another, e.g., DNA-binding domain of a polypeptide binding to DNA, at greater binding affinity than background binding. A binding domain “specifically binds” to a target site if it binds to or associates with a target site with an affinity or Kₐ (i.e., an equilibrium association constant of a particular binding interaction with units of 1/M) of, for example, greater than or equal to about 10⁵ M⁻¹. In certain embodiments, a binding domain binds to a target site with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (K_{d}) of a particular binding interaction with units of M (e.g., 10⁻⁵ M to 10⁻¹³ M, or less). Affinities of nuclease variants comprising one or more DNA-binding domains for DNA target sites contemplated in particular embodiments can be readily determined using conventional techniques, e.g., yeast cell surface display, or by binding association, or displacement assays using labeled ligands.

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

The terms “selectively binds” or “selectively bound” or “selectively binding” or “selectively targets” and describe preferential binding of one molecule to a target molecule (on-target binding) in the presence of a plurality of off-target molecules. In particular embodiments, an HE or megaTAL selectively binds an on-target DNA-binding site about 5, 10, 15, 20, 25, 50, 100, or 1000 times more frequently than the HE or megaTAL binds an off-target DNA target binding site.

“On-target” refers to a target site sequence.

“Off-target” refers to a sequence similar to but not identical to a target site sequence.

A “target site” or “target sequence” is a chromosomal or extrachromosomal nucleic acid sequence that defines a portion of a nucleic acid to which a binding molecule will bind and/or cleave, provided sufficient conditions for binding and/or cleavage exist. When referring to a polynucleotide sequence or SEQ ID NO. that references only one strand of a target site or target sequence, it would be understood that the target site or target sequence bound and/or cleaved by a nuclease variant is double-stranded and comprises the reference sequence and its complement. In various embodiments, the target site is in an immune system checkpoint gene, globin gene, gene that encodes a polypeptide that contributes to repression of γ-globin gene expression and/or HbF, or immunosuppressive signaling gene. In certain embodiments, the target site is a sequence in the human TRAC gene, CBL-B gene, or the PDCD1 gene.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair (HDR) mechanisms. This process requires nucleotide sequence homology, uses a “donor” molecule as a template to repair a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, polypeptides and nuclease variants, e.g., homing endonuclease variants, megaTALs, and related fusion polypeptides, contemplated herein are used for targeted double-stranded DNA cleavage. Endonuclease cleavage recognition sites may be on either DNA strand.

“Directionally biased” or “directionally biased deletion” refers to the location of deletions created by a cell's endogenous repair machinery in response to a double-strand DNA break induced by an endonuclease. If the deletions are directionally biased, they will predominately occur either 5′ or 3′ relative to the target cut site or double-strand DNA break (the breakpoint or breakpoint center). That is, substantially more deletions will occur on one side over the other, relative to the breakpoint center or target site center location. In particular embodiments, the fusion polypeptides induce genetic deletions having directionally bias deletions occurring on the same side as DNA-binding domain target site, relative to the breakpoint center or HE target site center location. In further embodiments, the fusion polypeptides induce genetic deletions having directionally biased deletion centers 5′ to that of the original breakpoint center or HE target site center location. In preferred embodiments, the directionally biased deletions are also elongated. Moreover, the amount of directionally biased deletions or elongation induced by the fusion proteins contemplated herein can be compared to the distribution of deletions or length of deletions produced by the co-expression of the same exonuclease and a fusion polypeptide comprising the same DNA-binding domain and same homing endonuclease.

An “exogenous” molecule is a molecule that is not normally present in a cell, but that is introduced into a cell by one or more genetic, biochemical or other methods. Exemplary exogenous molecules include, but are not limited to small organic molecules, protein, nucleic acid, carbohydrate, lipid, glycoprotein, lipoprotein, polysaccharide, any modified derivative of the above molecules, or any complex comprising one or more of the above molecules. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. Additional endogenous molecules can include proteins.

A “gene,” refers to a DNA region encoding a gene product, as well as all DNA regions which regulate the production of the gene product, whether or not such regulatory sequences are adjacent to coding and/or transcribed sequences. A gene includes, but is not limited to, promoter sequences, enhancers, silencers, insulators, boundary elements, terminators, polyadenylation sequences, post-transcription response elements, translational regulatory sequences such as ribosome binding sites and internal ribosome entry sites, replication origins, matrix attachment sites, and locus control regions.

“Gene expression” refers to the conversion of the information, contained in a gene, into a gene product. A gene product can be the direct transcriptional product of a gene (e.g., mRNA, tRNA, RNA, antisense RNA, ribozyme, structural RNA or any other type of RNA). Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “genetically engineered” or “genetically modified” refers to the chromosomal or extrachromosomal addition of extra genetic material in the form of DNA or RNA to the total genetic material in a cell. Genetic modifications may be targeted or non-targeted to a particular site in a cell's genome. In one embodiment, genetic modification is site-specific. In one embodiment, genetic modification is not site-specific.

As used herein, the term “genome editing” refers to the substitution, deletion, and/or introduction of genetic material at a target site in the cell's genome, which restores, corrects, disrupts, and/or modifies expression of a gene or gene product. Genome editing contemplated in particular embodiments comprises introducing one or more nuclease variants into a cell to generate DNA lesions at or proximal to a target site in the cell's genome, optionally in the presence of a donor repair template.

As used herein, the term “gene therapy” refers to the introduction of extra genetic material into the total genetic material in a cell that restores, corrects, or modifies expression of a gene or gene product, or for the purpose of expressing a therapeutic polypeptide. In particular embodiments, introduction of genetic material into the cell's genome by genome editing that restores, corrects, disrupts, or modifies expression of a gene or gene product, or for the purpose of expressing a therapeutic polypeptide is considered gene therapy.

### C. Fusion Polypeptides

Fusion polypeptides contemplated in particular embodiments herein that are suitable for editing a target site comprise a DNA-binding domain, a homing endonuclease, and an end-processing domain (e.g., an exonuclease). In various embodiments, the fusion polypeptides comprise a DNA-binding domain and a homing endonuclease variant, a polypeptide linker, and an exonuclease (e.g., ExoX), or biologically active fragment thereof. In particular embodiments, the fusion polypeptides comprise, in N-terminal to C-terminal order, a DNA-binding domain, a first linker domain, a homing endonuclease variant, a second linker domain, and an exonuclease (e.g., ExoX), or a biologically active fragment thereof.

In various embodiments, the DNA-binding domain is a TALE DNA-binding domain or a zinc finger DNA binding domain.

In various embodiments, the homing endonuclease variant is an engineered nuclease. The terms “reprogrammed nuclease,” “engineered nuclease,” or “nuclease variant” are used interchangeably and refer to a nuclease comprising one or more DNA-binding domains and one or more DNA cleavage domains (e.g., a nuclease or homing endonuclease), wherein the nuclease has been designed and/or modified from a parental or naturally occurring nuclease, to bind and cleave a double-stranded DNA target sequence or site. The nuclease variant may be designed and/or modified from a naturally occurring nuclease or from a previous nuclease variant. In some embodiments, the nuclease variant is designed to bind and cleave a non-native target sequence or site.

Illustrative examples of fusion polypeptides that bind and cleave a target sequence include, but are not limited to, megaTALs linked by a linker domain (e.g., a polypeptide linker) to an exonuclease (e.g., an ExoX exonuclease), or biologically active fragment thereof.

In preferred embodiments, the fusion polypeptides are useful for creating positionally biased and/or elongated deletions compared to deletions induced by similar non-fused/linked polypeptides, e.g., a separate megaTAL and exonuclease when introduced into a cell together. In particular embodiments, the deletions are more directionally biased compared to deletions induced by similar non-fused/linked polypeptides. In more particular embodiments, the fusion polypeptides contemplated herein create substantially more deletions on one side of a breakpoint over the other. For example, the described fusion polypeptides induce substantially more mutagenic deletions having a deletion center 5′ to the breakpoint or nuclease target site center location.

### 1. Homing Endonuclease (Meganuclease) Variants

In various embodiments, a homing endonuclease or meganuclease is reprogrammed to introduce double-strand breaks (DSBs) at a target site within a target gene. “Homing endonuclease” and “meganuclease” are used interchangeably and refer to naturally-occurring nucleases that recognize 12-45 base-pair cleavage sites (e.g., a target site) and are commonly grouped into five families based on sequence and structure motifs: LAGLIDADG, GIY-YIG, HNH, His-Cys box, and PD-(D/E)XK.

A “reference homing endonuclease” or “reference meganuclease” refers to a wild type homing endonuclease or a homing endonuclease found in nature. In one embodiment, a “reference homing endonuclease” refers to a wild type homing endonuclease that has been modified to increase basal activity.

An “engineered homing endonuclease,” “reprogrammed homing endonuclease,” “homing endonuclease variant,” “engineered meganuclease,” “reprogrammed meganuclease,” or “meganuclease variant” refers to a homing endonuclease comprising one or more DNA-binding domains and one or more DNA cleavage domains, wherein the homing endonuclease has been designed and/or modified from a parental or naturally occurring homing endonuclease, to bind and cleave a DNA target sequence or site. The homing endonuclease variant may be designed and/or modified from a naturally occurring homing endonuclease or from another homing endonuclease variant.

Homing endonuclease (HE) variants do not exist in nature and can be obtained by recombinant DNA technology or by random mutagenesis. HE variants may be obtained by making one or more amino acid alterations, e.g., mutating, substituting, adding, or deleting one or more amino acids, in a naturally occurring HE or HE variant. In particular embodiments, a HE variant comprises one or more amino acid alterations to the DNA recognition interface.

HE variants contemplated in particular embodiments may further comprise one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2, ExoI, or ExoX), 5′ flap endonuclease, helicase, template-dependent DNA polymerase or template-independent DNA polymerases activity. In various embodiments, polypeptides comprising an HE variant linked by a linker domain (e.g., a polypeptide linker) to an end-processing enzyme are provided. In various embodiments, the end-possessing enzyme exhibits 3′-5′ exonuclease activity. In particular embodiments, the end-processing enzyme is a Trex2, ExoI, or ExoX. The HE variant and end-processing enzyme may be introduced separately, e.g., in different vectors or separate mRNAs, or together, e.g., as a fusion protein, or in a polycistronic construct separated by a viral self-cleaving peptide or an IRES element.

A “DNA recognition interface” refers to the HE amino acid residues that interact with nucleic acid target bases as well as those residues that are adjacent. For each HE, the DNA recognition interface comprises an extensive network of side chain-to-side chain and side chain-to-DNA contacts, most of which is necessarily unique to recognize a particular nucleic acid target sequence. Thus, the amino acid sequence of the DNA recognition interface corresponding to a particular nucleic acid sequence varies significantly and is a feature of any natural or HE variant. By way of non-limiting example, a HE variant contemplated in particular embodiments may be derived by constructing libraries of HE variants in which one or more amino acid residues localized in the DNA recognition interface of the natural HE (or a previously generated HE variant) are varied. The libraries may be screened for target cleavage activity against each predicted BTK target site using cleavage assays (see e.g., Jarjour et al., 2009. Nuc. Acids Res. 37(20): 6871-6880).

LAGLIDADG homing endonucleases (LHE) are the most well studied family of homing endonucleases, are primarily encoded in archaea and in organellar DNA in green algae and fungi, and display the highest overall DNA recognition specificity. LHEs comprise one or two LAGLIDADG catalytic motifs per protein chain and function as homodimers or single chain monomers, respectively. Structural studies of LAGLIDADG proteins identified a highly conserved core structure (Stoddard 2005), characterized by an αββαββα fold, with the LAGLIDADG motif belonging to the first helix of this fold. The highly efficient and specific cleavage of LHEs represents a protein scaffold to derive novel, highly specific endonucleases. However, engineering LHEs to bind and cleave a non-natural or non-canonical target site requires selection of the appropriate LHE scaffold, examination of the target locus, selection of putative target sites, and extensive alteration of the LHE to alter its DNA contact points and cleavage specificity, at up to two-thirds of the base-pair positions in a target site.

In one embodiment, LHEs from which reprogrammed LHEs or LHE variants may be designed include, but are not limited to I-CreI and I-SceI.

Illustrative examples of LHEs from which reprogrammed LHEs or LHE variants may be designed include, but are not limited to I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

In one embodiment, the reprogrammed LHE or LHE variant is selected from the group consisting of: an I-CpaMI variant, an I-HjeMI variant, an I-OnuI variant, an I-PanMI variant, and an I-SmaMI variant.

In one embodiment, reprogrammed I-OnuI LHEs or I-OnuI variants targeting a target gene can be generated from a natural I-OnuI or biologically active fragment thereof (SEQ ID NOs: 102-106).

In various embodiments, target gene is an immune system checkpoint gene, a globin gene, a gene that encodes a polypeptide that contributes to repression of γ-globin gene expression and/or HbF, or an immunosuppressive signaling gene.

In one embodiment, the target gene is selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), TCRβ, IL10Rα, IL10Rβ, TGFBR1, TGFBR2, CBL-B, PCSK9, AHR, BTK, α-globin, β-globin, γ-globin, BCL11A, KLF1, SOX6, GATA1, LSD, alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), TEM5, TEM8, trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1) gene.

In certain embodiments, the target gene is selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), IL10Rα, TGFBR2, CBL-B, PCSK9, AHR, BTK, α-globin, β-globin, γ-globin, and BCL11A gene.

In certain embodiments, the target gene is TRAC (TCRα), CBL-B, or PDCD1 (PD-1) gene.

In one embodiment, the target gene is a TRAC gene, CBL-B gene, or PD-1 gene. In particular embodiments, the target gene/site comprises the nucleotide sequences set forth in SEQ ID NO: 1, 2, or 3.

In one embodiment, reprogrammed I-OnuI LHEs or I-OnuI variants targeting the human TRAC (TCRα) gene were generated from an existing I-OnuI variant. In some embodiments, reprogrammed I-OnuI LHEs were generated against a human TRAC gene target site set forth in SEQ ID NO: 1.

In another embodiment, reprogrammed I-OnuI LHEs or I-OnuI variants targeting the human PDCD1 (PD-1) gene were generated from an existing I-OnuI variant. In some embodiments, reprogrammed I-OnuI LHEs were generated against a human PD-1 gene target site set forth in SEQ ID NO: 2.

In another embodiment, reprogrammed I-OnuI LHEs or I-OnuI variants targeting the human CBL-B gene were generated from an existing I-OnuI variant. In some embodiments, reprogrammed I-OnuI LHEs were generated against a human CBL-B gene target site set forth in SEQ ID NO: 3.

In a particular embodiment, an I-OnuI LHE variant that binds and cleaves the a target gene comprises one or more amino acid substitutions or modifications in the DNA recognition interface of an I-OnuI as set forth in any one of SEQ ID NOs: 89, 92, 95, 98, 101 or 102-106, biologically active fragments thereof, and/or further variants thereof.

In a particular embodiment, the reprogrammed I-OnuI LHE or I-OnuI variant that binds and cleaves a target gene comprises one or more amino acid substitutions in the DNA recognition interface. In particular embodiments, the I-OnuI LHE that binds and cleaves a target gene comprises at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the DNA recognition interface of I-OnuI (Taekuchi et al. 2011. Proc Natl Acad Sci U.S.A. 2011 Aug. 9: 108(32): 13077-13082) or an I-OnuI LHE variant as set forth in SEQ ID NOs: 89, 92, 95, 98, 101 or 102-106, or further variants thereof.

In one embodiment, the I-OnuI LHE that binds and cleaves a target gene comprises at least 70%, more preferably at least 80%, more preferably at least 85%, more preferably at least 90%, more preferably at least 95%, more preferably at least 97%, more preferably at least 99% sequence identity with the DNA recognition interface of I-OnuI (Taekuchi et al. 2011. Proc Natl Acad Sci U.S.A 2011 Aug. 9; 108(32): 13077-13082) or an I-OnuI LHE variant as set forth in SEQ ID NOs: 89, 92, 95, 98, 101 or 102-106, or further variants thereof.

In a particular embodiment, an I-OnuI LHE variant that binds and cleaves the target gene comprises one or more amino acid substitutions or modifications in the DNA recognition interface, particularly in the subdomains situated from positions 24-50, 68-82, 180-203 and 223-240 of I-OnuI (SEQ ID NOs: 102-106) an I-OnuI variant as set forth in SEQ ID NOs: 89, 92, 95, 98, and 101, biologically active fragments thereof, and/or further variants thereof.

In a particular embodiment, an I-OnuI LHE variant that binds and cleaves the target gene comprises one or more amino acid substitutions or modifications in the DNA recognition interface at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of I-OnuI (SEQ ID NOs: 103-107) or an I-OnuI variant as set forth in SEQ ID NOs: 89, 92, 95, 98, and 101, biologically active fragments thereof, and/or further variants thereof.

In one embodiment, an I-OnuI LHE variant that binds and cleaves the target gene comprises one or more amino acid substitutions or modifications at additional positions situated anywhere within the entire I-OnuI sequence. The residues which may be substituted and/or modified include but are not limited to amino acids that contact the nucleic acid target or that interact with the nucleic acid backbone or with the nucleotide bases, directly or via a water molecule. In one non-limiting example a I-OnuI LHE variant contemplated herein that binds and cleaves the target gene comprises one or more substitutions and/or modifications, preferably at least 5, preferably at least 10, preferably at least 15, preferably at least 20, more preferably at least 25, more preferably at least 30, even more preferably at least 35, or even more preferably at least 40 in at least one position selected from the position group consisting of positions: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246, of I-OnuI SEQ ID NOs: 102-106 or an I-OnuI variant as set forth in SEQ ID NOs: 89, 92, 95, 98, and 101, biologically active fragments thereof, and/or further variants thereof.

In particular embodiments, an I-OnuI LHE variant that binds and cleaves the target gene comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of I-OnuI SEQ ID NOs: 102-106 or an I-OnuI variant as set forth in SEQ ID NOs: 89, 92, 95, 98, and 101, biologically active fragments thereof, and/or further variants thereof.

In particular embodiments, an I-OnuI LHE variant that binds and cleaves the target gene comprises an amino acid sequence that is at least 80%, preferably at least 85%, more preferably at least 90%, or even more preferably at least 95% identical to the amino acid sequence set forth in any one of SEQ ID NOs: 89, 92, 95, 98, and 101, or a biologically active fragment thereof.

In particular embodiments, an I-OnuI LHE variant comprises an amino acid sequence set forth in any one of SEQ ID NOs: 89, 92, 95, 98, and 101, or a biologically active fragment thereof.

In particular embodiments, an I-OnuI LHE variant comprises an amino acid sequence set forth in SEQ ID NO: 89, or a biologically active fragment thereof.

In particular embodiments, an I-OnuI LHE variant comprises an amino acid sequence set forth in SEQ ID NO: 92, or a biologically active fragment thereof.

In particular embodiments, an I-OnuI LHE variant comprises an amino acid sequence set forth in SEQ ID NO: 95, or a biologically active fragment thereof.

In particular embodiments, an I-OnuI LHE variant comprises an amino acid sequence set forth in SEQ ID NO: 98, or a biologically active fragment thereof.

In particular embodiments, an I-OnuI LHE variant comprises an amino acid sequence set forth in SEQ ID NO: 101, or a biologically active fragment thereof.

### 2. DNA-Binding Domains

In various embodiments, a fusion polypeptide contemplated herein, comprises a DNA-binding domain. In certain embodiments, the DNA-binding domain is located N-terminal to that of the homing endonuclease. In particular embodiments, the fusion polypeptide comprises a DNA-binding domain positioned N-terminal to that of a homing endonuclease, which is N-terminal to that of an end-processing domain (e.g., an exonuclease). In other words, the homing endonuclease is sandwiched between the DNA-binding domain and the exonuclease. Accordingly, in N-terminal to C-terminal order, an illustrative fusion polypeptide contemplated herein comprises a DNA-binding domain, a first polypeptide linker, a homing endonuclease, a second polypeptide linker, and an exonuclease.

In one aspect, the DNA-binding domain comprises a TALE DNA-binding domain. In particular embodiments, a fusion polypeptide comprises a megaTAL. A “megaTAL” refers to a polypeptide comprising a TALE DNA-binding domain and a homing endonuclease variant that binds and cleaves a DNA target sequence in a target gene, and further comprises one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2, ExoI or ExoX), 5′ flap endonuclease, helicase or template-independent DNA polymerases activity.

In various embodiments, a megaTAL comprising a homing endonuclease variant is reprogrammed to introduce double-strand breaks (DSBs) in a target gene. In some embodiments, a megaTAL comprising a homing endonuclease variant is reprogrammed to introduce a double-strand break in a target sequence in an immune system checkpoint gene, a globin gene, a gene that encodes a polypeptide that contributes to repression of γ-globin gene expression and/or HbF, or an immunosuppressive signaling gene. In some embodiments, a megaTAL comprising a homing endonuclease variant is reprogrammed to introduce a double-strand break in a target sequence in a human TRAC gene, PD-1 gene, or CBL-B gene (e.g., SEQ ID NOs: 1-3, respectively).

A “TALE DNA-binding domain” is the DNA-binding portion of transcription activator-like effectors (TALE or TAL-effectors), which mimics plant transcriptional activators to manipulate the plant transcriptome (see e.g., Kay et al., 2007. Science 318:648-651). TALE DNA-binding domains contemplated in particular embodiments are engineered de novo or from naturally occurring TALEs, e.g., AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brg11 and hpx17 from Ralstonia solanacearum. Illustrative examples of TALE proteins for deriving and designing DNA-binding domains are disclosed in U.S. Pat. No. 9,017,967, and references cited therein, all of which are incorporated herein by reference in their entireties.

In particular embodiments, the TALE DNA-binding domain comprising one or more repeat units that are involved in binding of the TALE DNA-binding domain to its corresponding target DNA sequence. A single “repeat unit” (also referred to as a “repeat”) is typically 33-35 amino acids in length. Each TALE DNA-binding domain repeat unit includes 1 or 2 DNA-binding residues making up the Repeat Variable Di-Residue (RVD), typically at positions 12 and/or 13 of the repeat. The natural (canonical) code for DNA recognition of these TALE DNA-binding domains has been determined such that an HD sequence at positions 12 and 13 leads to a binding to cytosine (C), NG binds to T, NI to A, NN binds to Gor A, and NG binds to T. In certain embodiments, non-canonical (atypical) RVDs are contemplated.

Illustrative examples of non-canonical RVDs suitable for use in particular megaTALs contemplated in particular embodiments include, but are not limited to HH, KH, NH, NK, NQ, RH, RN, SS, NN, SN, KN for recognition of guanine (G); NI, KI, RI, HI, SI for recognition of adenine (A); NG, HG, KG, RG for recognition of thymine (T); RD, SD, HD, ND, KD, YG for recognition of cytosine (C); NV, HN for recognition of A or G; and H*, HA, KA, N*, NA, NC, NS, RA, S* for recognition of A or T or G or C, wherein (*) means that the amino acid at position 13 is absent. Additional illustrative examples of RVDs suitable for use in particular megaTALs contemplated in particular embodiments further include those disclosed in U.S. Pat. No. 8,614,092, which is incorporated herein by reference in its entirety.

In particular embodiments, fusion polypeptide or megaTAL contemplated herein comprises a TALE DNA-binding domain comprising 3 to 30 repeat units. In certain embodiments, the TALE DNA-binding domain comprises 3, 4, 5, 6, 7, 8, 9, 10, 11, 12, 13, 14, 15, 16, 17, 18, 19, 20, 21, 22, 23, 24, 25, 26, 27, 28, 29, or 30 TALE DNA-binding domain repeat units. In a preferred embodiment, the TALE DNA-binding domain comprising 5-15 repeat units, more preferably 7-15 repeat units, more preferably 9-15 repeat units, and more preferably 9, 10, 11, 12, 13, 14, or 15 repeat units.

In particular embodiments, fusion polypeptide or a megaTAL contemplated herein comprises a TALE DNA-binding domain comprising 3 to 30 repeat units and an additional single truncated TALE repeat unit comprising 20 amino acids located at the C-terminus of a set of TALE repeat units, i.e., an additional C-terminal half-TALE DNA-binding domain repeat unit (amino acids −20 to −1 of the C-cap disclosed elsewhere herein, infra). Thus, in particular embodiments, a fusion polypeptide or megaTAL contemplated herein comprises a TALE DNA-binding domain comprising 3.5 to 30.5 repeat units. In certain embodiments, a fusion polypeptide or megaTAL comprises 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 TALE DNA-binding domain repeat units. In a preferred embodiment, a fusion polypeptide or megaTAL contemplated herein comprises a TALE DNA-binding domain comprising 5.5-15.5 repeat units, more preferably 7.5-15.5 repeat units, more preferably 9.5-15.5 repeat units, and more preferably 9.5, 10.5, 11.5, 12.5, 13.5, 14.5, or 15.5 repeat units.

In particular embodiments, a fusion polypeptide or megaTAL contemplated herein comprises a TAL effector architecture comprising an “N-terminal domain (NTD)” polypeptide, one or more TALE repeat domains/units, a “C-terminal domain (CTD)” polypeptide, and a homing endonuclease variant. In some embodiments, the NTD, TALE repeats, and/or CTD domains are from the same species. In other embodiments, one or more of the NTD, TALE repeats, and/or CTD domains are from different species.

As used herein, the term “N-terminal domain (NTD)” polypeptide refers to the sequence that flanks the N-terminal portion or fragment of a naturally occurring TALE DNA-binding domain. The NTD sequence, if present, may be of any length as long as the TALE DNA-binding domain repeat units retain the ability to bind DNA. In particular embodiments, the NTD polypeptide comprises at least 120 to at least 140 or more amino acids N-terminal to the TALE DNA-binding domain (0 is amino acid 1 of the most N-terminal repeat unit). In particular embodiments, the NTD polypeptide comprises at least about 120, 121, 122, 123, 124, 125, 126, 127, 128, 129, 130, 131, 132, 133, 134, 135, 136, 137, 138, 139, or at least 140 amino acids N-terminal to the TALE DNA-binding domain. In one embodiment, a fusion polypeptide or megaTAL contemplated herein comprises an NTD polypeptide of at least about amino acids +1 to +122 to at least about +1 to +137 of a Xanthomonas TALE protein (0) is amino acid 1 of the most N-terminal repeat unit). In particular embodiments, the NTD polypeptide comprises at least about 122, 123, 124, 125, 126, 127, 128, 129, 130, 131, 132, 133, 134, 135, 136, or 137 amino acids N-terminal to the TALE DNA-binding domain of a Xanthomonas TALE protein. In one embodiment, a fusion polypeptide or megaTAL contemplated herein comprises an NTD polypeptide of at least amino acids +1 to +121 of a Ralstonia TALE protein (0 is amino acid 1 of the most N-terminal repeat unit). In particular embodiments, the NTD polypeptide comprises at least about 121, 122, 123, 124, 125, 126, 127, 128, 129, 130, 131, 132, 133, 134, 135, 136, or 137 amino acids N-terminal to the TALE DNA-binding domain of a Ralstonia TALE protein.

As used herein, the term “C-terminal domain (CTD)” polypeptide refers to the sequence that flanks the C-terminal portion or fragment of a naturally occurring TALE DNA-binding domain. The CTD sequence, if present, may be of any length as long as the TALE DNA-binding domain repeat units retain the ability to bind DNA. In particular embodiments, the CTD polypeptide comprises at least 20 to at least 85 or more amino acids C-terminal to the last full repeat of the TALE DNA-binding domain (the first 20 amino acids are the half-repeat unit C-terminal to the last C-terminal full repeat unit). In particular embodiments, the CTD polypeptide comprises at least about 20, 21, 22, 23, 24, 25, 26, 27, 28, 29, 30, 31, 32, 33, 34, 35, 36, 37, 38, 39, 40, 41, 42, 443, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or at least 85 amino acids C-terminal to the last full repeat of the TALE DNA-binding domain. In one embodiment, a fusion polypeptide or megaTAL contemplated herein comprises a CTD polypeptide of at least about amino acids −20 to −1 of a Xanthomonas TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA-binding domain of a Xanthomonas TALE protein. In one embodiment, a fusion polypeptide or megaTAL contemplated herein comprises a CTD polypeptide of at least about amino acids −20 to −1 of a Ralstonia TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA-binding domain of a Ralstonia TALE protein.

In particular embodiments, a fusion polypeptide or megaTAL contemplated herein comprises a TALE DNA-binding domain engineered to bind a target sequence, a homing endonuclease reprogrammed to bind and cleave a target sequence/site, and optionally an NTD and/or CTD polypeptide, optionally joined to each other with one or more linker polypeptides contemplated elsewhere herein. It is further contemplated that a fusion polypeptide or megaTAL comprising TALE DNA-binding domain, and optionally an NTD and/or CTD polypeptide, may be fused to a linker polypeptide which is further fused to a homing endonuclease variant. Thus, the TALE DNA-binding domain binds a DNA target sequence that is within about 1, 2, 3, 4, 5, 6, 7, 8, 9, 10, 11, 12, 13, 14, or 15 nucleotides away from the target sequence bound by the DNA-binding domain of the homing endonuclease variant. In this way, the fusion polypeptides or megaTALs contemplated herein, increase the specificity and efficiency of genome editing.

In one embodiment, a fusion polypeptide or megaTAL comprises a homing endonuclease variant and a TALE DNA-binding domain that binds a nucleotide/target sequence/site that is within about 4, 5, or 6 nucleotides, preferably, 6 nucleotides upstream of the binding/target site of the reprogrammed homing endonuclease.

In particular embodiments, a fusion polypeptide or megaTAL contemplated herein, comprises one or more TALE DNA-binding repeat units and an LHE variant designed or reprogrammed from an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdi141I and variants thereof, or preferably I-CpaMI, I-HjeMI, I-OnuI, I-PanMI, SmaMI and variants thereof, or more preferably I-OnuI and variants thereof.

In particular embodiments, a fusion polypeptide or megaTAL contemplated herein, comprises an NTD, one or more TALE DNA-binding repeat units, a CTD, and an LHE variant selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdi141I and variants thereof, or preferably I-CpaMI, I-HjeMI, I-OnuI, I-PanMI, SmaMI and variants thereof, or more preferably I-OnuI and variants thereof.

In particular embodiments, a fusion polypeptide or megaTAL contemplated herein, comprises an NTD, about 9.5 to about 15.5 TALE DNA-binding repeat units, and an LHE variant selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdi141I and variants thereof, or preferably I-CpaMI, I-HjeMI, I-OnuI, I-PanMI, SmaMI and variants thereof, or more preferably I-OnuI and variants thereof.

In particular embodiments, a fusion polypeptide or megaTAL contemplated herein, comprises an NTD of about 122 amino acids to 137 amino acids, about 9.5, about 10.5, about 11.5, about 12.5, about 13.5, about 14.5, or about 15.5 binding repeat units, a CTD of about 20 amino acids to about 85 amino acids, and an I-OnuI LHE variant. In particular embodiments, any one of, two of, or all of the NTD, DNA-binding domain, and CTD can 25 be designed from the same species or different species, in any suitable combination.

In certain embodiments, a fusion polypeptide or megaTAL contemplated herein comprises, a TALE DNA-binding domain comprising about 9.5, about 10.5, about 11.5, about 12.5, about 13.5, about 14.5, or about 15.5 binding repeat units, a homing endonuclease variant contemplated elsewhere herein, and an end-processing domain (e.g., exonuclease), or biologically active fragment thereof, contemplated elsewhere herein.

In particular embodiments, a fusion polypeptide contemplated herein, comprises, in N-terminal to C-terminal order, a TALE DNA-binding domain comprising about 9.5, about 10.5, about 11.5, about 12.5, about 13.5, about 14.5, or about 15.5 binding repeat units, a first linker domain, an I-OnuI LHE variant, a second linker domain, and an end-processing enzyme (e.g., 3′ to 5′ exonuclease) or biologically active fragment thereof. In particular embodiments, any one of, two of, or all of the TALE binding repeats can be designed from the same species or different species, in any suitable combination. In preferred embodiments, the exonuclease is an ExoX exonuclease, or biologically active fragment thereof.

In particular embodiments, a fusion polypeptide or megaTAL contemplated herein comprises the amino acid sequence having at least 85%, 90%, 95%, 96%, 97%, 98%, or 99% identity to an amino acid sequence as set forth in any one of SEQ ID NOs: 7, 10, 58, 67, or 76. In particular embodiments, a fusion polypeptide or megaTAL contemplated herein comprises the amino acid sequence set forth in any one of SEQ ID NOs: 7, 10, 58, 67, or 76. In particular embodiments, a fusion polypeptide or megaTAL-Trex2 fusion protein contemplated herein comprises an amino acid sequence having at least 85%, 90%, 95%, 96%, 97%, 98%, or 99% identity to an amino acid sequence as set forth in any one of SEQ ID NO: 13, 61, 70, or 79. In particular embodiments, a fusion polypeptide or megaTAL-Trex2 fusion protein contemplated herein, comprises the amino acid sequence set forth in any one of SEQ ID NO: 13, 61, 70, or 79. In particular embodiments, a fusion polypeptide or megaTAL-ExoX fusion protein contemplated herein, comprises an amino acid sequence having at least 85%, 90%, 95%, 96%, 97%, 98%, or 99% identity to an amino acid sequences as set forth in any one of SEQ ID NO: 46, 64, 73, or 82. In particular embodiments, a fusion polypeptide or megaTAL-ExoX fusion protein contemplated herein comprises the amino acid sequence set forth in any one of SEQ ID NO: 46, 64, 73, or 82. In particular embodiments, a fusion polypeptide or megaTAL-ExoI fusion protein contemplated herein comprises an amino acid sequence having at least 85%, 90%, 95%, 96%, 97%, 98%, or 99% identity to an amino acid sequence as set forth in SEQ ID NO: 43. In particular embodiments, a fusion polypeptide or megaTAL-ExoI fusion protein contemplated herein comprises the amino acid sequence set forth in SEQ ID NO: 43.

In certain embodiments, a fusion polypeptide or megaTAL contemplated herein, is encoded by an mRNA sequence having at least 85%, 90%, 95%, 96%, 97%, 98%, or 99% identity to a mRNA sequence as set forth in any one of SEQ ID NO: 6, 9, 57, 66, or 75. In certain embodiments, a fusion polypeptide or megaTAL contemplated herein, is encoded by an mRNA sequence set forth in any one of SEQ ID NO: 6, 9, 57, 66, or 75. In certain embodiments, a fusion polypeptide or megaTAL-Trex2 contemplated herein, is encoded by an mRNA sequence having at least 85%, 90%, 95%, 96%, 97%, 98%, or 99% identity to a mRNA sequence as set forth in any one of SEQ ID NO: 12, 60, 69, or 78. In certain embodiments, a fusion polypeptide or megaTAL-Trex2 contemplated herein, is encoded by an mRNA sequence set forth in any one of SEQ ID NO: 12, 60, 69, or 78. In certain embodiments, a fusion polypeptide or megaTAL-ExoX contemplated herein, is encoded by an mRNA sequence having at least 85%, 90%, 95%, 96%, 97%, 98%, or 99% identity to a mRNA sequence as set forth in any one of SEQ ID NO: 45, 63, 72, or 81. In certain embodiments, a fusion polypeptide or megaTAL-ExoX contemplated herein, is encoded by an mRNA sequence set forth in any one of SEQ ID NO: 45, 63, 72, or 81. In certain embodiments, a fusion polypeptide or megaTAL-ExoI contemplated herein, is encoded by an mRNA sequence having at least 85%, 90%, 95%, 96%, 97%, 98%, or 99% identity to a mRNA sequence as set forth in SEQ ID NO: 42. In certain embodiments, a fusion polypeptide or megaTAL-ExoI contemplated herein, is encoded by an mRNA sequence set forth in SEQ ID NO: 42.

In certain embodiments, a fusion polypeptide or megaTAL contemplated herein, is encoded by a DNA sequence having at least 85%, 90%, 95%, 96%, 97%, 98%, or 99% identity to a DNA sequence as set forth in any one of SEQ ID NO: 5, 8, 56, 65, or 74. In certain embodiments, a fusion polypeptide or megaTAL contemplated herein, is encoded by a DNA sequence set forth in any one of SEQ ID NO: 5, 8, 56, 65, or 74. In certain embodiments, a fusion polypeptide or megaTAL-Trex2 contemplated herein, is encoded by a DNA sequence having at least 85%, 90%, 95%, 96%, 97%, 98%, or 99% identity to a DNA sequence as set forth in any one of SEQ ID NO: 11, 59, 68, or 77. In certain embodiments, a fusion polypeptide or megaTAL-Trex2 contemplated herein, is encoded by a DNA sequence set forth in any one of SEQ ID NO: 11, 59, 68, or 77. In certain embodiments, a fusion polypeptide or megaTAL-ExoX contemplated herein, is encoded by a DNA sequence having at least 85%, 90%, 95%, 96%, 97%, 98%, or 99% identity to a DNA sequence as set forth in any one of SEQ ID NO: 44, 62, 71, or 80. In certain embodiments, a fusion polypeptide or megaTAL-ExoX contemplated herein, is encoded by a DNA sequence set forth in any one of SEQ ID NO: 44, 62, 71, or 80. In certain embodiments, a fusion polypeptide or megaTAL-ExoI contemplated herein, is encoded by a DNA sequence having at least 85%, 90%, 95%, 96%, 97%, 98%, or 99% identity to a DNA sequence as set forth in SEQ ID NO: 41. In certain embodiments, a fusion polypeptide or megaTAL-ExoI contemplated herein, is encoded by a DNA sequence set forth in SEQ ID NO: 41.

In certain embodiments, a megaTAL comprises a TALE DNA-binding domain and an I-OnuI LHE variant the binds and cleaves the nucleotide sequence set forth in any one of SEQ ID NOs: 1-3.

In another aspect, the DNA-binding domain comprises a zinc finger DNA-binding domain. In particular embodiments, the fusion polypeptides contemplated herein, comprise a zinc finger DNA-binding domain, a homing endonuclease domain contemplated elsewhere herein, and an end-processing domain (e.g., exonuclease) contemplated elsewhere herein.

In particular embodiments, the zinc finger DNA-binding domain that has one, two, three, four, five, six, seven, or eight or more zinc finger motifs. Typically, a single zinc finger motif is about 30 amino acids in length. Zinc fingers motifs include both canonical C₂H₂ zinc fingers, and non-canonical zinc fingers such as, for example, C₃H zinc fingers and C₄ zinc fingers.

Zinc finger DNA-binding domains can be engineered to bind any DNA sequence. Candidate zinc finger DNA-binding domains for a given 3 bp DNA target sequence have been identified and modular assembly strategies have been devised for linking a plurality of the domains into a multi-finger peptide targeted to the corresponding composite DNA target sequence. Other suitable methods known in the art can also be used to design and construct nucleic acids encoding zinc finger DNA-binding domains, e.g., phage display, random mutagenesis, combinatorial libraries, computer/rational design, affinity selection, PCR, cloning from cDNA or genomic libraries, synthetic construction and the like. (See, e.g., U.S. Pat. No. 5,786,538; Wu et al, PNAS 92:344-348 (1995); Jamieson et al, Biochemistry 33:5689-5695 (1994); Rebar & Pabo, Science 263:671-673 (1994); Choo & Klug, PNAS 91:11163-11167 (1994); Choo & Klug, PNAS 9 1: 11168-1 1172 (1994); Desjarlais & Berg, PNAS 90:2256-2260 (1993); Desjarlais & Berg, PNAS 89:7345-7349 (1992); Pomerantz et al, Science 267:93-96 (1995); Pomerantz et al, PNAS 92:9752-9756 (1995); Liu et al, PNAS 94:5525-5530 (1997); Griesman & Pabo, Science 275:657-661 (1997); Desjarlais & Berg, PNAS 91: 1 1-99-1 1103 (1994)).

Individual zinc finger motifs bind to a three or four nucleotide sequence. The length of a sequence to which a zinc finger binding domain is engineered to bind (e.g., a target sequence) will determine the number of zinc finger motifs in an engineered zinc finger DNA-binding domain. For example, when the zinc finger motifs do not bind to overlapping subsites, a six-nucleotide target sequence is bound by a two-finger DNA-binding domain; a nine-nucleotide target sequence is bound by a three-finger DNA-binding domain, etc. In particular embodiments, DNA-binding sites for individual zinc fingers motifs in a target site need not be contiguous, but can be separated by one or several nucleotides, depending on the length and nature of the linker sequences between the zinc finger motifs in a multi-finger binding domain.

In certain embodiments, a fusion polypeptide contemplated herein, comprises a zinc finger DNA-binding domain comprising one or more zinc finger motifs, a linker, a homing endonuclease variant, a linker, and an end-processing domain (e.g., exonuclease). In particular embodiments, the homing endonuclease is a LHE variant LHE variant designed or reprogrammed from an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdi141I and variants thereof, or preferably I-CpaMI, I-HjeMI, I-OnuI, I-PanMI, SmaMI and variants thereof, or more preferably I-OnuI and variants thereof.

In certain embodiments, fusion polypeptides contemplated herein comprise, a zinc finger DNA-binding domain comprising 2, 3, 4, 5, 6, 7, or 8 or more zinc finger motifs, a homing endonuclease variant contemplated elsewhere herein, and an end-processing domain (e.g., exonuclease), or biologically active fragment thereof, contemplated elsewhere herein.

In particular embodiments, a fusion polypeptide contemplated herein, comprises, in N-terminal to C-terminal order, a zinc finger DNA binding domain comprising about 2, 3, 4, 5, 6, 7, or 8 or more zinc finger motifs, a first linker domain, an I-OnuI LHE variant, a second linker domain, and an end-processing domain (e.g., exonuclease) or biologically active fragment thereof. In particular embodiments, any one of, two of, or all of the zinc finger motifs can be designed from the same species or different species, in any suitable combination. In preferred embodiments, the exonuclease is an ExoX exonuclease, or biologically active fragment thereof.

### 3. End-Processing Enzymes

Genome editing compositions (e.g., fusion polypeptides) and methods contemplated in particular embodiments comprise editing cellular genomes using a DNA-binding domain, a homing endonuclease variant, and an end-processing enzyme. In particular embodiments, a fusion polypeptide encodes a DNA-binding domain, a homing endonuclease variant and one or more end-processing enzymes (e.g., exonucleases), each separated by a linker domain (e.g., a peptide linker).

The term “end-processing enzyme” refers to an enzyme that modifies the exposed ends of a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An end-processing enzyme may modify exposed polynucleotide chain ends by adding one or more nucleotides, removing one or more nucleotides, removing or modifying a phosphate group and/or removing or modifying a hydroxyl group. An end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents.

In particular embodiments, genome editing compositions and methods contemplated in particular embodiments comprise editing cellular genomes using a fusion polypeptide comprising a DNA-binding domain, a homing endonuclease variant, and a DNA end-processing enzyme.

The term “DNA end-processing enzyme” refers to an enzyme that modifies the exposed ends of DNA. A DNA end-processing enzyme may modify blunt ends or staggered ends (ends with 5′ or 3′ overhangs). A DNA end-processing enzyme may modify single stranded or double stranded DNA. A DNA end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents. DNA end-processing enzyme may modify exposed DNA ends by adding one or more nucleotides, removing one or more nucleotides, removing or modifying a phosphate group and/or removing or modifying a hydroxyl group.

Illustrative examples of DNA end-processing enzymes suitable for use in particular embodiments contemplated herein include, but are not limited to: 5′-3′ exonucleases, 5′-3′ alkaline exonucleases, 3′-5′ exonucleases, 5′ flap endonucleases, helicases, phosphatases, hydrolases and template-independent DNA polymerases.

Additional illustrative examples of DNA end-processing enzymes suitable for use in particular embodiments contemplated herein include, but are not limited to, Trex2, Trex1, Trex1 without transmembrane domain, Apollo, Artemis, DNA2, ExoI, ExoT, ExoIII, ExoX, Fen1, Fan1, MreII, Rad2, Rad9, TdT (terminal deoxynucleotidyl transferase), PNKP, RecE, RecJ, RecQ, Lambda exonuclease, Sox, Vaccinia DNA polymerase, exonuclease I, exonuclease III, exonuclease VII, NDK1, NDK5, NDK7, NDK8, WRN, T7-exonuclease Gene 6, avian myeloblastosis virus integration protein (IN), Bloom, Antartic Phophatase, Alkaline Phosphatase, Poly nucleotide Kinase (PNK), ApeI, Mung Bean nuclease, Hex1, TTRAP (TDP2), Sgs1, Sae2, CUP, Pol mu, Pol lambda, MUS81, EME1, EME2, SLX1, SLX4 and UL-12.

In particular embodiments, genome editing compositions and methods for editing cellular genomes contemplated herein comprise fusion polypeptides comprising a DNA-binding domain, a homing endonuclease variant, and an exonuclease. The term “exonuclease” refers to enzymes that cleave phosphodiester bonds at the end of a polynucleotide chain via a hydrolyzing reaction that breaks phosphodiester bonds at either the 3′ or 5′ end. In particular embodiments, the exonuclease is a 3′-5′ exonuclease. In some embodiments, the exonuclease is an ExoX exonuclease, or biologically active fragment thereof. In some embodiments, the exonuclease is an ExoI exonuclease, or biologically active fragment thereof. In some embodiments, the exonuclease is a Trex2 exonuclease, or biologically active fragment thereof.

ExoX is an 3′-5′ distributive exonuclease from Escherichia coli (E. coli) and a member of the DnaQ superfamily. ExoX is also referred to as Exodeoxyribonuclease 10, Exodeoxyribonuclease X, Exonuclease X, and Exo X. Exemplary ExoX reference sequence numbers used in particular embodiments include, but are not limited to: NP_416358.1; NC_000913.3; WP_000944256.1; NZ_STEB01000009.1AAC74914.

In preferred embodiments contemplated herein, a fusion polypeptide comprises a DNA-binding domain, a homing endonuclease variant, and an ExoX exonuclease, or a biologically active fragment thereof. In various embodiments, the fusion polypeptide comprises, a DNA-binding domain and a homing endonuclease variant, linked by a linker domain (e.g., a polypeptide linker) to an ExoX exonuclease, or a biologically active fragment thereof.

In various embodiments, the ExoX comprises an amino acid sequence having at least 85%, 90%, 95%, 96%, 97%, 98%, or 99% identity to an amino acid sequence as set forth in SEQ ID NO: 109. In some embodiments, the ExoX comprises an amino acid sequence having at least 85% identity to an amino acid sequence as set forth in SEQ ID NO: 109. In some embodiments, the ExoX comprises an amino acid sequence having at least 90% identity to an amino acid sequence as set forth in SEQ ID NO: 109. In some embodiments, the ExoX comprises an amino acid sequence having at least 95% identity to an amino acid sequence as set forth in SEQ ID NO: 109. In some embodiments, the ExoX comprises an amino acid sequence having at least 96% identity to an amino acid sequence as set forth in SEQ ID NO: 109. In some embodiments, the ExoX comprises an amino acid sequence having at least 97% identity to an amino acid sequence as set forth in SEQ ID NO: 109. In some embodiments, the ExoX comprises an amino acid sequence having at least 98% identity to an amino acid sequence as set forth in SEQ ID NO: 109. In some embodiments, the ExoX comprises an amino acid sequence having at least 99% identity to an amino acid sequence as set forth in SEQ ID NO: 109.

In particular embodiments, the ExoX, or biologically active fragment thereof, comprises an amino acid an amino acid sequence as set forth in SEQ ID NO: 109.

ExoI is an 3′-5′ processive exonuclease from Escherichia coli (E. coli) and a member of the DnaQ superfamily. ExoI is also referred to as Exodeoxyribonuclease I, Exonuclease I, DNA deoxyribophosphodiesterase, and dRPase. Exemplary ExoI reference sequence numbers used in particular embodiments include, but are not limited to: NP_416515.1, NC_000913.3, WP_000980589.1, NZ_LN832404.1.

In preferred embodiments contemplated herein, a fusion polypeptide comprises a DNA-binding domain, a homing endonuclease variant, and an ExoI exonuclease, or a biologically active fragment thereof. In various embodiments, the fusion polypeptide comprises, a DNA-binding domain and a homing endonuclease variant, linked by a linker domain (e.g., a polypeptide linker) to an ExoI exonuclease, or a biologically active fragment thereof.

In various embodiments, the ExoI comprises an amino acid sequence having at least 85%, 90%, 95%, 96%, 97%, 98%, or 99% identity to an amino acid sequence as set forth in SEQ ID NO: 112. In some embodiments, the ExoI comprises an amino acid sequence having at least 85% identity to an amino acid sequence as set forth in SEQ ID NO: 112. In some embodiments, the ExoI comprises an amino acid sequence having at least 90% identity to an amino acid sequence as set forth in SEQ ID NO: 112. In some embodiments, the ExoI comprises an amino acid sequence having at least 95% identity to an amino acid sequence as set forth in SEQ ID NO: 112. In some embodiments, the ExoI comprises an amino acid sequence having at least 96% identity to an amino acid sequence as set forth in SEQ ID NO: 112. In some embodiments, the ExoI comprises an amino acid sequence having at least 97% identity to an amino acid sequence as set forth in SEQ ID NO: 112. In some embodiments, the ExoI comprises an amino acid sequence having at least 98% identity to an amino acid sequence as set forth in SEQ ID NO: 112. In some embodiments, the ExoI comprises an amino acid sequence having at least 99% identity to an amino acid sequence as set forth in SEQ ID NO: 112.

In particular embodiments, the ExoI, or biologically active fragment thereof, comprises an amino acid an amino acid sequence as set forth in SEQ ID NO: 112.

### D. Target Sites

Homing endonuclease variants contemplated in particular embodiments can be designed to bind to any suitable target sequence (e.g., a sequence within the human genome) and can have a novel binding specificity, compared to a naturally-occurring nuclease. In particular embodiments, the target site is a regulatory region of a gene including, but not limited to promoters, enhancers, repressor elements, and the like. In particular embodiments, the target site is a coding region of a gene or a splice site. In particular embodiments, a nuclease variant and donor repair template can be designed to insert a therapeutic polynucleotide. In particular embodiments, a nuclease variant and donor repair template can be designed to insert a therapeutic polynucleotide under control of endogenous gene regulatory elements or expression control sequences. In various embodiments, nuclease variants bind to and cleave a target sequence in a immune system checkpoint gene, globin gene, gene that encodes a polypeptide that contributes to repression of γ-globin gene expression and/or HbF, or immunosuppressive signaling gene.

Illustrative examples of immune system checkpoint genes include, but are not limited to: PD-1, LAG-3, TIM-3, CTLA-4, BTLA, TIGIT, VISTA, and KIRs.

Illustrative examples of genes encoding immunosuppressive signaling components include, but are not limited to: IL-10Rα, TGFBR1, TGFBR2, AHR, SGK1, TSC2, VHL, A2AR, and CBL-B.

Illustrative examples of polypeptides that repress γ-globin gene expression and HbF include, but are not limited to: BCL11A, KLF1, SOX6, GATA1, and LSD1.

In various embodiments, nuclease variants bind to and cleave a target sequence in a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), TCRβ, IL10Rα, IL10Rβ, TGFBR1, TGFBR2, CBL-B, PCSK9, AHR, BTK, α-globin, β-globin, γ-globin, BCL11A, KLF1, SOX6, GATA1, LSD, alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), TEM5, TEM8, trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1) gene.

In certain embodiments, the target gene is selected from the group consisting of: programmed cell death protein 1 (PD-1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), IL10Rα, TGFBR2, CBL-B, PCSK9, AHR, BTK, α-globin, β-globin, γ-globin, and BCL11A gene.

In certain embodiments, the target gene is TRAC (TCRα), CBL-B, or PDCD1 (PD-1) gene.

In various embodiments, nuclease variants bind to and cleave a target sequence in the TRAC gene. T-cell receptor alpha (TRAC) locus is a protein that in humans is encoded by the TRA gene. TRAC is also referred to as TRA, IMD7, TCRA, TRA@, TRAC, T-cell receptor alpha locus, TCRD, T cell receptor alpha locus, TCRα. It contributes the alpha chain to the larger TCR protein (T-cell receptor). Alpha-beta T cell receptors are antigen specific receptors which are essential to the immune response and are present on the cell surface of T lymphocytes. They recognize peptide-major histocompatibility (MH) (pMH) complexes that are displayed by antigen presenting cells (APC), a prerequisite for efficient T cell adaptive immunity against pathogens.

In particular embodiments, a nuclease variant introduces a DSB in exon 1 of the constant region of the human TCRα gene, preferably at SEQ ID NO: 1 in exon 1 of the constant region of the human TCRα gene, and more preferably at the sequence “ATTC” in SEQ ID NO: 1 in exon 1 of the constant region of the human TCRα gene. In a preferred embodiment, the TCRα gene is a human TCRα gene.

In various embodiments, the homing endonuclease variants bind to and cleave a target sequence in a program death receptor 1 (PD-1) gene. PD-1 is also referred to as programmed cell death 1 (PDCD1), systemic lupus erythematosus susceptibility 2 (SLEB2), CD279, HPD1, PD1, HPD-L, and HSLE1. PD-1 is a member of the B7/CD28 family of costimulatory receptors. The PD-1 molecule consists of an extracellular ligand binding IgV domain, a transmembrane domain, and an intracellular domain which has potential phosphorylation sites located with immune tyrosine-based inhibitory motif (ITIM) and immune receptor inhibitory tyrosine-based switch motif (ITSM). PD-1 is an inhibitory co-receptor expressed on T cells, Tregs, exhausted T cells, B cells, activated monocytes, dendritic cells (DCs), natural killer (NK) cells and natural killer T (NKT) cells. PD-1 negatively regulates T-cell activation through binding to its ligands, programmed death ligand 1 (PD-L1) and programmed death ligand 2 (PD-L2). PD-1 binding inhibits T-cell proliferation, and interferon-γ (IFN-γ), tumor necrosis factor-a, and IL-2 production, and reduces T-cell survival. PD-1 expression is a hallmark of “exhausted” T cells that have experienced high levels of stimulation. This state of exhaustion, which occurs during chronic infections and cancer, is characterized by T-cell dysfunction, resulting in suboptimal control of infections and tumors.

In particular embodiments, a homing endonuclease variant introduces a double-strand break (DSB) in exon 1 of a PD-1 gene, preferably at SEQ ID NO: 2 in exon 1 of a PD-1 gene, and more preferably at the sequence “ATCC” in SEQ ID NO: 2 in exon 1 of a PD-1 gene. In a preferred embodiment, a homing endonuclease variant or megaTAL cleaves double-stranded DNA and introduces a DSB into the polynucleotide sequence set forth in SEQ ID NO: 2. In a preferred embodiment, the PD-1 gene is a human PD-1 gene.

In various embodiments, homing endonuclease variants bind to and cleave a target sequence in a human casitas B-lineage (Cbl) lymphoma proto-oncogene B (CBLB) gene. CBL is also referred to as CBL2; Noonan syndrome-like disorder with or without juvenile myelomonocytic leukemia (NSLL); C-CBL; RING finger protein 55 (RNF55); fragile site, folic acid type, rare, fra (11) (q23.3) (FRA11B); E3 ubiquitin-protein ligase CBL; Cas-Br-M (murine) ecotropic retroviral transforming sequence; Cbl proto-oncogene, E3 ubiquitin protein ligase; RING-type E3 ubiquitin transferase CBL; casitas B-lineage lymphoma proto-oncogene; oncogene CBL2; proto-oncogene c-Cbl; and signal transduction protein CBL.

This gene is a proto-oncogene that encodes a RING finger E3 ubiquitin ligase. The encoded protein is one of the enzymes required for targeting substrates for degradation by the proteasome. This protein mediates the transfer of ubiquitin from ubiquitin conjugating enzymes (E2) to specific substrates. This protein also contains an N-terminal phosphotyrosine binding domain that allows it to interact with numerous tyrosine-phosphorylated substrates and target them for proteasome degradation. CBLB functions as a negative regulator of many signal transduction pathways, including T cell activation and persistence.

In particular embodiments, a homing endonuclease variant introduces a double-strand break (DSB) in a target site in a CBLB gene. In particular embodiments, a homing endonuclease variant or megaTAL introduces a DSB in exon 6 of a CBLB gene, preferably at SEQ ID NO: 3 in exon 6 of a CBLB gene, and more preferably at the sequence “ATTC” in SEQ ID NO: 3 in exon 6 of a CBLB gene. In a preferred embodiment, a homing endonuclease variant cleaves double-stranded DNA and introduces a DSB into the polynucleotide sequence set forth in SEQ ID NO: 3. In a preferred embodiment, the CBLB gene is a human CBLB gene.

### E. Donor Repair Templates

Fusion polypeptides, including homing endonuclease variants, may be used to introduce a DSB in a target sequence; the DSB may be repaired through homology directed repair (HDR) mechanisms in the presence of one or more donor repair templates. In particular embodiments, the donor repair template is used to insert a sequence into the genome. In particular preferred embodiments, the donor repair template is used to insert a polynucleotide sequence encoding a therapeutic polypeptide. In particular preferred embodiments, the donor repair template is used to insert a polynucleotide sequence encoding a therapeutic polypeptide, such that the expression of the polypeptide is under control of the endogenous promoter and/or enhancers.

In various embodiments, a donor repair template is introduced into a hematopoietic cell, e.g., a hematopoietic stem or progenitor cell, or CD34⁺ cell, by transducing the cell with an adeno-associated virus (AAV), retrovirus, e.g., lentivirus, IDLV, etc., herpes simplex virus, adenovirus, or vaccinia virus vector comprising the donor repair template.

In particular embodiments, the donor repair template comprises one or more homology arms that flank the DSB site.

As used herein, the term “homology arms” refers to a nucleic acid sequence in a donor repair template that is identical, or nearly identical, to DNA sequence flanking the DNA break introduced by the nuclease at a target site. In one embodiment, the donor repair template comprises a 5′ homology arm that comprises a nucleic acid sequence that is identical or nearly identical to the DNA sequence 5′ of the DNA break site. In one embodiment, the donor repair template comprises a 3′ homology arm that comprises a nucleic acid sequence that is identical or nearly identical to the DNA sequence 3′ of the DNA break site. In a preferred embodiment, the donor repair template comprises a 5′ homology arm and a 3′ homology arm. The donor repair template may comprise homology to the genome sequence immediately adjacent to the DSB site, or homology to the genomic sequence within any number of base pairs from the DSB site. In one embodiment, the donor repair template comprises a nucleic acid sequence that is homologous to a genomic sequence about 5 bp, about 10 bp, about 25 bp, about 50 bp, about 100 bp, about 250 bp, about 500 bp, about 1000 bp, about 2500 bp, about 5000 bp, about 10000 bp or more, including any intervening length of homologous sequence.

Illustrative examples of suitable lengths of homology arms contemplated in particular embodiments, may be independently selected, and include but are not limited to: about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, about 1600 bp, about 1700 bp, about 1800 bp, about 1900 bp, about 2000 bp, about 2100 bp, about 2200 bp, about 2300 bp, about 2400 bp, about 2500 bp, about 2600 bp, about 2700 bp, about 2800 bp, about 2900 bp, or about 3000 bp, or longer homology arms, including all intervening lengths of homology arms.

Additional illustrative examples of suitable homology arm lengths include, but are not limited to: about 100 bp to about 3000 bp, about 200 bp to about 3000 bp, about 300 bp to about 3000 bp, about 400 bp to about 3000 bp, about 500 bp to about 3000 bp, about 500 bp to about 2500 bp, about 500 bp to about 2000 bp, about 750 bp to about 2000 bp, about 750 bp to about 1500 bp, or about 1000 bp to about 1500 bp, including all intervening lengths of homology arms.

In a particular embodiment, the lengths of the 5′ and 3′ homology arms are independently selected from about 500 bp to about 1500 bp. In one embodiment, the 5′homology arm is about 1500 bp and the 3′ homology arm is about 1000 bp. In one embodiment, the 5′homology arm is from about 200 bp to about 600 bp and the 3′ homology arm is from about 200 bp to about 600 bp. In one embodiment, the 5′homology arm is about 200 bp and the 3′ homology arm is about 200 bp. In one embodiment, the 5′homology arm is about 300 bp and the 3′ homology arm is about 300 bp. In one embodiment, the 5′homology arm is about 400 bp and the 3′ homology arm is about 400 bp. In one embodiment, the 5′homology arm is about 500 bp and the 3′ homology arm is about 500 bp. In one embodiment, the 5′homology arm is about 600 bp and the 3′ homology arm is about 600 bp.

### F. Polypeptides

Various polypeptides are contemplated herein, including, but not limited to, fusion polypeptides, including homing endonuclease variants and/or megaTALs. In preferred embodiments, a polypeptide comprises the amino acid sequence set forth in any one of SEQ ID NOs: 7, 10, 13, 19, 22, 25, 28, 31, 34, 37, 40, 43, 46, 49, 52, 55, 58, 61, 64, 67, 70, 73, 76, 79, 82, 89, 92, 95, 98, and 101. “Polypeptide,” “polypeptide fragment,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well-known recombinant and/or synthetic techniques. Polypeptides are not limited to a specific length, e.g., they may comprise a full-length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring.

An “isolated protein,” “isolated peptide,” or “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances.

Illustrative examples of polypeptides contemplated in particular embodiments include, but are not limited to homing endonuclease variants, megaTALs, end-processing nucleases, exonucleases, fusion polypeptides and variants thereof.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to improve the biological properties of a fusion polypeptide, homing endonuclease, megaTAL or the like that binds and cleaves a target site by introducing one or more substitutions, deletions, additions and/or insertions into the polypeptide. In particular embodiments, polypeptides include polypeptides having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to any of the reference sequences contemplated herein, typically where the variant maintains at least one biological activity of the reference sequence.

Polypeptides variants include biologically active “polypeptide fragments.” Illustrative examples of biologically active polypeptide fragments include DNA-binding domains, nuclease domains, endo-processing domains (e.g., exonucleases) and the like. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In preferred embodiments, the biological activity is binding affinity and/or cleavage activity for a target sequence. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long. In particular embodiments, a polypeptide comprises a biologically active fragment of a homing endonuclease variant. In particular embodiments, the polypeptides set forth herein may comprise one or more amino acids denoted as “X.” “X” if present in an amino acid SEQ ID NO, refers to any amino acid. One or more “X” residues may be present at the N- and C-terminus of an amino acid sequence set forth in particular SEQ ID NOs contemplated herein. If the “X” amino acids are not present the remaining amino acid sequence set forth in a SEQ ID NO may be considered a biologically active fragment.

In particular embodiments, a polypeptide comprises a biologically active fragment of a homing endonuclease variant, e.g., SEQ ID NOs: 89, 92, 95, 98, or 101, or a megaTAL (e.g., SEQ ID NOs: 7, 10, 58, 67, and 76). The biologically active fragment may comprise an N-terminal truncation and/or C-terminal truncation. In a particular embodiment, a biologically active fragment lacks or comprises a deletion of the 1, 2, 3, 4, 5, 6, 7, or 8 N-terminal amino acids of a homing endonuclease variant compared to a corresponding wild type homing endonuclease sequence, more preferably a deletion of the 4 N-terminal amino acids of a homing endonuclease variant compared to a corresponding wild type homing endonuclease sequence. In a particular embodiment, a biologically active fragment lacks or comprises a deletion of the 1, 2, 3, 4, or 5 C-terminal amino acids of a homing endonuclease variant compared to a corresponding wild type homing endonuclease sequence, more preferably a deletion of the 2 C-terminal amino acids of a homing endonuclease variant compared to a corresponding wild type homing endonuclease sequence. In a particular preferred embodiment, a biologically active fragment lacks or comprises a deletion of the 4 N-terminal amino acids and 2 C-terminal amino acids of a homing endonuclease variant compared to a corresponding wild type homing endonuclease sequence.

In a particular embodiment, an I-OnuI variant comprises a deletion of 1, 2, 3, 4, 5, 6, 7, or 8 the following N-terminal amino acids: M, A, Y, M, S. R. R. E; and/or a deletion of the following 1, 2, 3, 4, or 5 C-terminal amino acids: R, G, S, F, V.

In a particular embodiment, an I-OnuI variant comprises a deletion or substitution of 1, 2, 3, 4, 5, 6, 7, or 8 the following N-terminal amino acids: M, A, Y, M, S. R. R, E; and/or a deletion or substitution of the following 1, 2, 3, 4, or 5 C-terminal amino acids: R, G, S, F, V.

In a particular embodiment, an I-OnuI variant comprises a deletion of 1, 2, 3, 4, 5, 6, 7, or 8 the following N-terminal amino acids: M, A, Y, M, S, R, R, E; and/or a deletion of the following 1 or 2 C-terminal amino acids: F, V.

In a particular embodiment, an I-OnuI variant comprises a deletion or substitution of 1, 2, 3, 4, 5, 6, 7, or 8 the following N-terminal amino acids: M, A, Y, M, S, R, R, E; and/or a deletion or substitution of the following 1 or 2 C-terminal amino acids: F, V.

As noted above, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154; 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments, polypeptides include polypeptides having at least about and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |

| | | | | | | |
|---|---|---|---|---|---|---|
| Threonine | T | Thr | ACA | ACC | ACG | ACU |
| Valine | V | Val | GUA | GUC | GUG | GUU |

| Tryptophan | W | Trp | UGG | |
|---|---|---|---|---|
| Tyrosine | Y | Tyr | UAC | UAU |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g. Watson et al. Molecular Biology of the Gene, 4th Edition, 1987, The Benjamin/Cummings Pub. Co., p. 224).

In one embodiment, where expression of two or more polypeptides is desired, the polynucleotide sequences encoding them can be separated by and IRES sequence as disclosed elsewhere herein.

Polypeptides contemplated in particular embodiments include fusion polypeptides, e.g., SEQ ID NOs: 7, 10, 13, 19, 22, 25, 28, 31, 34, 37, 40, 43, 46, 49, 52, 55, 58, 61, 64, 67, 70, 73, 76, 79, and 82. In preferred embodiments, the fusion polypeptide comprises an amino acid sequence as set forth in any one of SEQ ID NOs; and 46, 64, 73, and 82. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

In another embodiment, two or more polypeptides can be expressed as a fusion protein that comprises one or more self-cleaving polypeptide sequences as disclosed elsewhere herein.

In one embodiment, a fusion protein contemplated herein comprises one or more DNA-binding domains and one or more nucleases, and one or more linker and/or self-cleaving polypeptides.

In one embodiment, a fusion protein contemplated herein comprises a nuclease variant; a linker or self-cleaving peptide; and an end-processing enzyme including but not limited to a 5′-3′ exonuclease, a 5′-3′ alkaline exonuclease, and a 3′-5′ exonuclease (e.g., Trex2, ExoI, or ExoX).

Fusion polypeptides can comprise one or more polypeptide domains or segments including, but are not limited to signal peptides, cell permeable peptide domains (CPP), DNA-binding domains, nuclease domains, etc., epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. In particular embodiments, the polypeptides of the fusion protein can be in any order. Fusion polypeptides or fusion proteins can also include conservatively modified variants, polymorphic variants, alleles, mutants, subsequences, and interspecies homologs, so long as the desired activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as disclosed elsewhere herein.

Fusion polypeptides may optionally comprise a linker that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: glycine polymers (G)ₙ; glycine-serine polymers (G₁₋₅S₁₋₅)ₙ, where n is an integer of at least one, two, three, four, or five; glycine-alanine polymers; alanine-serine polymers; GGG (SEQ ID NO: 113); DGGGS (SEQ ID NO: 114); TGEKP (SEQ ID NO: 115) (see e.g., Liu et al., PNAS 5525-5530 (1997)); GGRR (SEQ ID NO: 116) (Pomerantz et al. 1995, supra); (GGGGS)ₙ wherein n=1, 2, 3, 4 or 5 (SEQ ID NOs: 117 and 150-153) (Kim et al., PNAS 93, 1156-1160 (1996.); EGKSSGSGSESKVD (SEQ ID NO: 118) (Chaudhary et al., 1990, Proc. Natl. Acad. Sci. U.S.A. 87:1066-1070); KESGSVSSEQLAQFRSLD (SEQ ID NO: 119) (Bird et al., 1988, Science 242:423-426), GGRRGGGS (SEQ ID NO: 120) LRQRDGERP (SEQ ID NO: 121); LRQKDGGGSERP (SEQ ID NO: 122); LRQKD(GGGS)₂ERP (SEQ ID NO: 123). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein or between an endogenous open reading frame and a polypeptide encoded by a donor repair template. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5 (8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picomna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 121), for example, ENLYFQG (SEQ ID NO: 122) and ENLYFQS (SEQ ID NO: 123), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 2.

TABLE 2
Exemplary 2A sites include the
following sequences:

| | |
|---|---|
| SEQ ID NO: 127 | GSGATNFSLLKQAGDVEENPGP |
| SEQ ID NO: 128 | ATNFSLLKQAGDVEENPGP |
| SEQ ID NO: 129 | LLKQAGDVEENPGP |
| SEQ ID NO: 130 | GSGEGRGSLLTCGDVEENPGP |
| SEQ ID NO: 131 | EGRGSLLTCGDVEENPGP |
| SEQ ID NO: 132 | LLTCGDVEENPGP |
| SEQ ID NO: 133 | GSGQCTNYALLKLAGDVESNPGP |
| SEQ ID NO: 134 | QCTNYALLKLAGDVESNPGP |
| SEQ ID NO: 135 | LLKLAGDVESNPGP |
| SEQ ID NO: 136 | GSGVKQTLNFDLLKLAGDVESNPG |
| | P |
| SEQ ID NO: 137 | VKQTLNFDLLKLAGDVESNPGP |
| SEQ ID NO: 138 | LLKLAGDVESNPGP |
| SEQ ID NO: 139 | LLNFDLLKLAGDVESNPGP |
| SEQ ID NO: 140 | TLNFDLLKLAGDVESNPGP |
| SEQ ID NO: 141 | LLKLAGDVESNPGP |
| SEQ ID NO: 142 | NFDLLKLAGDVESNPGP |
| SEQ ID NO: 143 | QLLNFDLLKLAGDVESNPGP |
| SEQ ID NO: 144 | APVKQTLNFDLLKLAGDVESNPGP |
| SEQ ID NO: 145 | VTELLYRMKRAETYCPRPLLAIHP |
| | TEARHKQKIVAPVKQT |
| SEQ ID NO: 146 | LNFDLLKLAGDVESNPGP |
| SEQ ID NO: 147 | LLAIHPTEARHKQKIVAPVKQTLN |
| | FDLLKLAGDVESNPGP |
| SEQ ID NO: 148 | EARHKQKIVAPVKQTLNFDLLKLA |
| | GDVESNPGP |

### G. Polynucleotides

In particular embodiments, polynucleotides encoding one or more fusion polypeptides, homing endonuclease variants, megaTALs, end-processing enzymes, and exonucleases contemplated herein are provided. As used herein, the terms “polynucleotide” or “nucleic acid” refer to deoxyribonucleic acid (DNA), ribonucleic acid (RNA) and DNA/RNA hybrids.

In various embodiments, a polynucleotide encoding one or more fusion polypeptides, e.g., SEQ ID NOs: 5, 8, 11, 17, 20, 23, 26, 29, 32, 35, 38, 41, 44, 47, 50, 53, 56, 59, 62, 65, 68, 71, 74, 77, and 80. In some embodiments, the polynucleotide encoding one or more fusion polypeptides is an RNA polynucleotide. In some embodiments, the RNA polynucleotide encoding one or more fusion polypeptides comprises a sequence set forth in any one of SEQ ID NOs: 6, 9, 12, 18, 21, 24, 27, 30, 33, 36, 39, 42, 45, 48, 51, 54, 57, 60, 63, 66, 69, 72, 75, 78, and 81. In some embodiments, the polynucleotide encodes an amino acid sequence as set forth in any one of SEQ ID NOs: 7, 10, 13, 19, 22, 25, 28, 31, 34, 37, 40, 43, 46, 49, 52, 55, 58, 61, 64, 67, 70, 73, 76, 79, and 82.

Polynucleotides may be single-stranded or double-stranded and either recombinant, synthetic, or isolated. Polynucleotides include, but are not limited to: pre-messenger RNA (pre-mRNA), messenger RNA (mRNA), synthetic RNA, synthetic mRNA, genomic DNA (gDNA), PCR amplified DNA, complementary DNA (cDNA), synthetic DNA, and recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths.” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid, and/or (xi) isolated removal of spurious translation initiation sites.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose. Exemplary natural nitrogenous bases include the purines, adenosine (A) and guanidine (G), and the pyrimidines, cytidine (C) and thymidine (T) (or in the context of RNA, uracil (U)). The C-1 atom of deoxyribose is bonded to N-1 of a pyrimidine or N-9 of a purine. Nucleotides are usually mono, di- or triphosphates. The nucleotides can be unmodified or modified at the sugar, phosphate and/or base moiety, (also referred to interchangeably as nucleotide analogs, nucleotide derivatives, modified nucleotides, non-natural nucleotides, and non-standard nucleotides; see for example, WO 92/07065 and WO 93/15187). Examples of modified nucleic acid bases are summarized by Limbach et al., (1994, Nucleic Acids Res. 22, 2183-2196).

A nucleotide may also be regarded as a phosphate ester of a nucleoside, with esterification occurring on the hydroxyl group attached to C-5 of the sugar. As used herein, the term “nucleoside” refers to a heterocyclic nitrogenous base in N-glycosidic linkage with a sugar. Nucleosides are recognized in the art to include natural bases, and also to include well known modified bases. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, nucleoside derivatives, modified nucleosides, non-natural nucleosides, or non-standard nucleosides). As also noted above, examples of modified nucleic acid bases are summarized by Limbach et al., (1994, Nucleic Acids Res. 22, 2183-2196).

Illustrative examples of polynucleotides include, but are not limited to polynucleotides encoding SEQ ID NOs: 7, 10, 13, 16, 19, 22, 25, 28, 31, 34, 37, 40, 43, 46, 49, 52, 55, 58, 61, 64, 67, 70, 73, 76, 79, 82, 89, 92, 95, 98, and 101 and polynucleotide sequences set forth in SEQ ID NOs: 5, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 81, 87, 88, 90, 91, 93, 94, 96, 97, 99, and 100.

In various illustrative embodiments, polynucleotides contemplated herein include, but are not limited to polynucleotides encoding fusion polypeptides, homing endonuclease variants, megaTALs, end-processing enzymes, exonucleases, and expression vectors, viral vectors, and transfer plasmids comprising polynucleotides contemplated herein.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg. His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window;” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc., 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant polynucleotide, a synthetic polynucleotide, or other polynucleotide that does not exist in nature and that has been made by the hand of man.

In various embodiments, a polynucleotide comprises an mRNA encoding a polypeptide contemplated herein including, but not limited to, a fusion polypeptide, homing endonuclease variant, a megaTAL, an end-processing enzyme, and an exonuclease. In certain embodiments, the mRNA comprises a cap, one or more nucleotides and/or modified nucleotides, and a poly(A) tail.

In particular embodiments, an mRNA contemplated herein comprises a poly(A) tail to help protect the mRNA from exonuclease degradation, stabilize the mRNA, and facilitate translation. In certain embodiments, an mRNA comprises a 3′ poly(A) tail structure.

In particular embodiments, the length of the poly(A) tail is at least about 10, 25, 50, 75, 100, 150, 200, 250, 300, 350, 400, 450, or at least about 500 or more adenine nucleotides or any intervening number of adenine nucleotides. In particular embodiments, the length of the poly(A) tail is at least about 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, or 275 or more adenine nucleotides.

In particular embodiments, the length of the poly(A) tail is about 10 to about 500 adenine nucleotides, about 50 to about 500 adenine nucleotides, about 100 to about 500 adenine nucleotides, about 150 to about 500 adenine nucleotides, about 200 to about 500 adenine nucleotides, about 250 to about 500 adenine nucleotides, about 300 to about 500 adenine nucleotides, about 50 to about 450 adenine nucleotides, about 50 to about 400 adenine nucleotides, about 50 to about 350 adenine nucleotides, about 100 to about 500 adenine nucleotides, about 100 to about 450 adenine nucleotides, about 100 to about 400 adenine nucleotides, about 100 to about 350 adenine nucleotides, about 100 to about 300 adenine nucleotides, about 150 to about 500 adenine nucleotides, about 150 to about 450 adenine nucleotides, about 150 to about 400 adenine nucleotides, about 150 to about 350 adenine nucleotides, about 150 to about 300 adenine nucleotides, about 150 to about 250 adenine nucleotides, about 150 to about 200 adenine nucleotides, about 200 to about 500 adenine nucleotides, about 200 to about 450 adenine nucleotides, about 200 to about 400 adenine nucleotides, about 200 to about 350 adenine nucleotides, about 200 to about 300 adenine nucleotides, about 250 to about 500 adenine nucleotides, about 250 to about 450 adenine nucleotides, about 250 to about 400 adenine nucleotides, about 250 to about 350 adenine nucleotides, or about 250 to about 300 adenine nucleotides or any intervening range of adenine nucleotides.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (pre-mRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G TC A T G 3′ is 3′T CA G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette contains the sequence of a therapeutic gene used to treat, prevent, or ameliorate a genetic disorder. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, as contemplated herein.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

In a certain embodiment, a polynucleotide-of-interest comprises a donor repair template.

The polynucleotides contemplated in particular embodiments, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, post-transcription response elements, e.g., Woodchuck Hepatitis Virus post-transcriptional response element (WPRE), Hepatitis B Virus post-transcriptional response element (HPRE), and polynucleotides encoding self-cleaving polypeptides, epitope tags, as disclosed elsewhere herein or as known in the art, such that their overall length may vary considerably. It is therefore contemplated in particular embodiments that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector. A desired polypeptide can also be expressed by delivering an mRNA encoding the polypeptide into the cell.

Illustrative examples of vectors include, but are not limited to plasmid, autonomously replicating sequences, and transposable elements, e.g., Sleeping Beauty, Piggy Bac.

Additional illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

“Expression control sequences,” “control elements,” or “regulatory sequences” present in an expression vector are those non-translated regions of the vector-origin of replication, selection cassettes, promoters, enhancers, translation initiation signals (Shine Dalgarno sequence or Kozak sequence) introns, post-transcriptional regulatory elements, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide comprises a vector, including but not limited to expression vectors and viral vectors. A vector may comprise one or more exogenous, endogenous, or heterologous control sequences such as promoters and/or enhancers. An “endogenous control sequence” is one which is naturally linked with a given gene in the genome. An “exogenous control sequence” is one which is placed in juxtaposition to a gene by means of genetic manipulation (i.e., molecular biological techniques) such that transcription of that gene is directed by the linked enhancer/promoter. A “heterologous control sequence” is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular therapy.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, a short elongation factor 1-alpha (EF1a-short) promoter, a long elongation factor 1-alpha (EF1a-long) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Virol. 69 (2):748-55 (1995)).

In a particular embodiment, it may be desirable to use a cell, cell type, cell lineage or tissue specific expression control sequence to achieve cell type specific, lineage specific, or tissue specific expression of a desired polynucleotide sequence (e.g., to express a particular nucleic acid encoding a polypeptide in only a subset of cell types, cell lineages, or tissues or during specific stages of development).

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments, polynucleotides comprise at least one (typically two) site(s) for recombination mediated by a site-specific recombinase. As used herein, the terms “recombinase” or “site-specific recombinase” include excisive or integrative proteins, enzymes, co-factors or associated proteins that are involved in recombination reactions involving one or more recombination sites (e.g., two, three, four, five, six, seven, eight, nine, ten or more.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The polynucleotides may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site-specific recombination site” refer to a particular nucleic acid sequence to which a recombinase recognizes and binds.

In particular embodiments, polynucleotides contemplated herein, include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See. e.g., Jackson et al., 1990. Trends Biochem Sci 15 (12):477-83) and Jackson and Kaminski. 1995. RNA 1 (10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18 (11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66 (3): 1602-9) and the VEGF IRES (Huez et al., 1998. Mol Cell Biol 18 (11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In particular embodiments, the polynucleotides comprise polynucleotides that have a consensus Kozak sequence and that encode a desired polypeptide. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 149), where R is a purine (A or G) (Kozak, 1986. Cell. 44(2):283-92, and Kozak, 1987. Nucleic Acids Res. 15(20):8125-48).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “poly A site” or “polyA sequence” as used herein denotes a DNA sequence which directs both the termination and polyadenylation of the nascent RNA transcript by RNA polymerase II. Polyadenylation sequences can promote mRNA stability by addition of a poly A tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal poly A sequence (e.g., AATAAA, ATTAAA, AGTAAA). In particular embodiments, the poly(A) sequence is an SV40 poly A sequence, a bovine growth hormone poly A sequence (BGHpA), a rabbit β-globin poly A sequence (rβgpA), variants thereof, or another suitable heterologous or endogenous poly A sequence known in the art.

In particular embodiments, polynucleotides encoding one or more fusion polypeptides, homing endonuclease variants, megaTALs, end-processing enzymes, or exonucleases may be introduced into hematopoietic cells, e.g., CD34⁺ cells, by both non-viral and viral methods. In particular embodiments, delivery of one or more polynucleotides encoding nucleases and/or donor repair templates may be provided by the same method or by different methods, and/or by the same vector or by different vectors.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a CD34⁺ cell.

Illustrative examples of non-viral vectors include, but are not limited to plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

In particular embodiments, polynucleotides (e.g., DNA or RNA) encoding the fusion polypeptides contemplated herein can be introduced directly into the cells, for example by electroporation. In electroporation methods, the polynucleotides, or the complexes of site-directed polypeptides and polynucleotides are mixed in an electroporation buffer with the target cells to form a suspension. This suspension is then subjected to an electrical pulse at an optimized voltage, which creates temporary pores in the phospholipid bilayer of the cell membrane, permitting charged molecules like DNA and proteins to be driven through the pores and into the cell. Reagents and equipment to perform electroporation are sold commercially. The electroporation medium can be any suitable medium known in the art. Suitable methods of electroporation are available and known to those of skill in the art

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors comprising nuclease variants and/or donor repair templates are administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include, but are not limited to adeno-associated virus (AAV), retrovirus, herpes simplex virus, adenovirus, and vaccinia virus vectors.

### H. Genome Edited Cells

The genome edited cells manufactured and/or edited by the methods contemplated in particular embodiments are provided. Genome edited cells contemplated in particular embodiments may be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are obtained from a mammalian subject. In a more preferred embodiment, the cells are obtained from a primate subject, optionally a non-human primate. In the most preferred embodiment, the cells are obtained from a human subject.

An “isolated cell” refers to a non-naturally occurring cell, e.g., a cell that does not exist in nature, a modified cell, an engineered cell, etc. . . . that has been obtained from an in vivo tissue or organ and is substantially free of extracellular matrix.

In particular embodiments, the cells are edited by the fusion polypeptides contemplated herein, e.g., a fusion polypeptide comprising a DNA-binding domain and a homing endonuclease (HE) variant that binds and cleaves a selected double strand DNA (dsDNA) target site in a cell, a polypeptide linker and an exonuclease, or biologically active fragment thereof.

In various embodiments, the cell comprises directionally biased deletions induced by the fusion polypeptides contemplated herein. In some embodiments, at least 50%, at least 55%, at least 60%, at least 65%, at least 70%, at least 75%, or at least 80% of the directionally biased deletions have a deletion center location on one side of the HE target site center location. In some embodiments, the deletion center location is on the same side as the DNA-binding domain target site relative to the HE target site center location. In some embodiments, the deletion center location is 5′ to the HE target site center location.

In various embodiments, at least 50%, at least 55%, at least 60%, at least 65%, at least 70%, at least 75%, or at least 80% of deletions have a deletion center greater than 4 nucleotides away from the HE target site center location. In various embodiments, at least 10%, at least 15%, at least 20%, at least 25%, at least 30%, or at least 35% of deletions have a deletion center greater than 8 nucleotides away from the HE target site center location.

In various embodiments, at least 50%, at least 55%, at least 60%, at least 65%, at least 70%, at least 75%, or at least 80% of deletions are 6 bps in length or greater. In various embodiments, at least 30%, at least 31%, at least 32%, at least 33%, at least 34%, at least 35%, at least 40%, at least 55%, or at least 60% of deletions are 12 bps in length or greater. In particular embodiments, the directionally biased deletions comprise a length of about 10, about 11, about 12, about 13, about 14, about 15, about 16, about 17, about 18, about 19, or about 20 nucleotides.

In various embodiments, the deletion extends into the DNA-binding domain target site. In some embodiments, the deletion center location is within the DNA-binding domain target site.

Illustrative examples of cell types whose genome can be edited using the compositions and methods contemplated herein include, but are not limited to, cell lines, primary cells, stem cells, progenitor cells, and differentiated cells.

The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Symmetric cell division produces two identical daughter cells. “Proliferation” or “expansion” of cells refers to symmetrically dividing cells.

As used herein, the term “progenitor” or “progenitor cells” refers to cells have the capacity to self-renew and to differentiate into more mature cells. Many progenitor cells differentiate along a single lineage, but may have quite extensive proliferative capacity.

In particular embodiments, the cell is a primary cell. The term “primary cell” as used herein is known in the art to refer to a cell that has been isolated from a tissue and has been established for growth in vitro or ex vivo. Corresponding cells have undergone very few, if any, population doublings and are therefore more representative of the main functional component of the tissue from which they are derived in comparison to continuous cell lines, thus representing a more representative model to the in vivo state. Methods to obtain samples from various tissues and methods to establish primary cell lines are well-known in the art (see, e.g., Jones and Wise, Methods Mol Biol. 1997). Primary cells for use in the methods contemplated herein are derived from umbilical cord blood, placental blood, mobilized peripheral blood and bone marrow. In one embodiment, the primary cell is a hematopoietic stem or progenitor cell.

In one embodiment, the genome edited cell is an embryonic stem cell. In one embodiment, the genome edited cell is an adult stem or progenitor cell. In one embodiment, the genome edited cell is primary cell.

In a preferred embodiment, the genome edited cell is a hematopoietic cell, e.g., hematopoietic stem cell, hematopoietic progenitor cell, such as a B cell progenitor cell, or cell population comprising hematopoietic cells.

As used herein, the term “population of cells” refers to a plurality of cells that may be made up of any number and/or combination of homogenous or heterogeneous cell types, as described elsewhere herein. For example, for transduction of hematopoietic stem or progenitor cells, a population of cells may be isolated or obtained from umbilical cord blood, placental blood, bone marrow, or mobilized peripheral blood. A population of cells may comprise about 10%, about 20%, about 30%, about 40%, about 50%, about 60%, about 70%, about 80%, about 90%, or about 100% of the target cell type to be edited. In certain embodiments, hematopoietic stem or progenitor cells may be isolated or purified from a population of heterogeneous cells using methods known in the art.

Illustrative sources to obtain hematopoietic cells include, but are not limited to: cord blood, bone marrow or mobilized peripheral blood.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells can repopulate the erythroid, neutrophil-macrophage, megakaryocyte and lymphoid hematopoietic cell pool.

Additional illustrative examples of hematopoietic stem or progenitor cells suitable for use with the methods and compositions contemplated herein include hematopoietic cells that are CD34⁺CD38^{Lo}D90⁺CD45^{RA-}, hematopoietic cells that are CD34⁺, CD59⁺, Thy1/CD90⁺, CD38^{Lo/-}, C-kit/CD117⁺, and Lin⁽⁻⁾, and hematopoietic cells that are CD133⁺.

In a preferred embodiment, the hematopoietic cells that are CD133⁺CD90⁺. In a preferred embodiment, the hematopoietic cells that are CD133⁺CD34⁺. In a preferred embodiment, the hematopoietic cells that are CD133⁺CD90⁺CD34⁺.

The term, “CD34+ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34+ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Various methods exist to characterize hematopoietic hierarchy. One method of characterization is the SLAM code. The SLAM (Signaling lymphocyte activation molecule) family is a group of >10 molecules whose genes are located mostly tandemly in a single locus on chromosome 1 (mouse), all belonging to a subset of immunoglobulin gene superfamily, and originally thought to be involved in T-cell stimulation. This family includes CD48, CD150, CD244, etc., CD150 being the founding member, and, thus, also called slamF1, i.e., SLAM family member 1. The signature SLAM code for the hematopoietic hierarchy is hematopoietic stem cells (HSC)—CD150⁺CD48⁻CD244⁻; multipotent progenitor cells (MPPs)—CD150⁻CD48⁻CD244⁺; lineage-restricted progenitor cells (LRPs)—CD150⁻CD48⁺CD244⁺; common myeloid progenitor (CMP)—lin-SCA-1-c-kit⁺CD34⁺CD16/32^{mid}; granulocyte-macrophage progenitor (GMP)—lin SCA-1-c-kit⁺CD34⁺CD16/32^{hi}; and megakaryocyte-erythroid progenitor (MEP)—lin SCA-1-c-kit⁺CD34⁻CD16/32^{low}.

Preferred target cell types edited with the compositions and methods contemplated herein include, hematopoietic cells, preferably human hematopoietic cells, more preferably human hematopoietic stem and progenitor cells, and even more preferably CD34⁺ human hematopoietic stem cells. The term “CD34+ cell,” as used herein refers to a cell expressing the CD34 protein on its cell surface. “CD34,” as used herein refers to a cell surface glycoprotein (e.g., sialomucin protein) that often acts as a cell-cell adhesion factor. CD34+ is a cell surface marker of both hematopoietic stem and progenitor cells.

In one embodiment, the genome edited hematopoietic cells are CD150⁺CD48⁻ CD244⁻ cells. In one embodiment, the genome edited hematopoietic cells are CD34⁺CD133⁺ cells. In one embodiment, the genome edited hematopoietic cells are CD133⁺ cells. In one embodiment, the genome edited hematopoietic cells are CD34⁺ cells.

In particular embodiments, the fusion polypeptides contemplated herein are introduced and expressed in immune effector cells. An “immune effector cell,” is any cell of the immune system that has one or more effector functions (e.g., cytotoxic cell killing activity, secretion of cytokines, induction of ADCC and/or CDC). Illustrative immune effector cells contemplated herein are T lymphocytes, including but not limited to cytotoxic T cells (CTLs; CD8+ T cells), TILs, and helper T cells (HTLs; CD4+ T cells). In a particular embodiment, the cells comprise αβ T cells. In a particular embodiment, the cells comprise γδ T cells. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are autologous.

Illustrative immune effector cells used with the fusion polypeptides contemplated in particular embodiments include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), CD4+CD8+ T cell, CD4−CD8− T cell, or any other subset of T cells. Other illustrative populations of T cells suitable for use in particular embodiments include naïve T cells (TN), T memory stem cells (TSCM), central memory T cells (TCM), effector memory T cells (TEM), and effector T cells (TEFF).

As would be understood by the skilled person, other cells may also be used as immune effector cells with the fusion polypeptides herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoietic stem cells (HSCs) contained within the CD34+ population of cells derived from cord blood, bone marrow or mobilized peripheral blood which upon administration in a subject differentiate into mature immune effector cells, or which can be induced in vitro to differentiate into mature immune effector cells.

Methods for making the immune effector cells that express a CAR contemplated herein are provided in particular embodiments. In one embodiment, the method comprises transfecting or transducing immune effector cells isolated from an individual such that the immune effector cells express one or more CARs contemplated herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein).

In particular embodiments, prior to in vitro manipulation or genetic modification of the immune effector cells contemplated herein, the source of cells is obtained from a subject. In particular embodiments, modified immune effector cells comprise T cells.

In particular embodiments, PBMCs may be directly genetically modified to express a CAR using methods contemplated herein. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the chimeric antigen receptors contemplated herein (e.g., transduced with a viral vector comprising a nucleic acid encoding a CAR or a polycistronic message encoding a CAR) and then are activated and expanded in vitro. In various embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In one embodiment, CD34+ cells are transduced with a nucleic acid construct contemplated herein. In certain embodiments, the transduced CD34+ cells differentiate into mature immune effector cells in vivo following administration into a subject, generally the subject from whom the cells were originally isolated. In another embodiment, CD34+ cells may be stimulated in vitro prior to exposure to or after being genetically modified with a CAR as contemplated herein, with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In particular embodiments, a population of modified immune effector cells for the treatment of cancer comprises a CAR contemplated herein. For example, a population of modified immune effector cells are prepared from peripheral blood mononuclear cells (PBMCs) obtained from a patient diagnosed with B cell malignancy described herein (autologous donors). The PBMCs form a heterogeneous population of T lymphocytes that can be CD4+, CD8+, or CD4+ and CD8+.

The PBMCs also can include other cytotoxic lymphocytes such as NK cells or NKT cells. An expression vector carrying the coding sequence of a CAR contemplated in particular embodiments is introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR protein expressing T cells in addition to cell activation using anti-CD3 antibodies and or anti-CD28 antibodies and IL-2 or any other methods known in the art as described elsewhere herein. Standard procedures are used for cryopreservation of T cells expressing the CAR protein T cells for storage and/or preparation for use in a human subject. In one embodiment, the in vitro transduction, culture and/or expansion of T cells are performed in the absence of non-human animal derived products such as fetal calf serum and fetal bovine serum. Since a heterogeneous population of PBMCs is genetically modified, the resultant transduced cells are a heterogeneous population of modified cells comprising a BCMA targeting CAR as contemplated herein.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells.

Genetically engineered cells, including T cells, can be manufactured using various methods known in the art, see, e.g., WO 2016/094304 which is incorporated herein by reference in its entirety.

### I. Compositions and Formulations

The compositions contemplated in particular embodiments may comprise one or more polypeptides, polynucleotides, vectors comprising same, and genome editing compositions and genome edited cell compositions, as contemplated herein. The genome editing compositions and methods contemplated in particular embodiments are useful for editing a target site in a cell or a population of cells.

In various embodiments, the compositions contemplated herein comprise a fusion polypeptide comprising a DNA-binding domain, a homing endonuclease variant, and an end-processing enzyme, e.g., a 3′-5′ exonuclease (ExoX). The nuclease variant may be in the form of an mRNA that is introduced into a cell via polynucleotide delivery methods disclosed supra, e.g., electroporation, lipid nanoparticles, etc. In one embodiment, a composition comprising an mRNA encoding a fusion polypeptide, homing endonuclease variant, and a 3′-5′ exonuclease (e.g., ExoX), is introduced in a cell via polynucleotide delivery methods disclosed supra.

In particular embodiments, the compositions contemplated herein comprise a population of cells, a nuclease variant, and optionally, a donor repair template. In particular embodiments, the compositions contemplated herein comprise a population of cells, a nuclease variant, an end-processing enzyme, and optionally, a donor repair template. The nuclease variant and/or end-processing enzyme may be in the form of an mRNA that is introduced into the cell via polynucleotide delivery methods disclosed supra. The donor repair template may also be introduced into the cell by means of a separate composition.

In particular embodiments, the compositions contemplated herein comprise a population of cells, a fusion polypeptide comprising a DNA-binding domain, a homing endonuclease variant, an 3′-5′ exonuclease (e.g., ExoX) and optionally, a donor repair template. The fusion polypeptide comprising a DNA-binding domain, a homing endonuclease variant, and 3′-5′ exonuclease may be in the form of an mRNA that is introduced into the cell via polynucleotide delivery methods disclosed supra. The donor repair template may also be introduced into the cell by means of a separate composition.

In particular embodiments, the population of cells comprise genetically modified hematopoietic cells including, but not limited to, hematopoietic stem cells, hematopoietic progenitor cells, CD133⁺ cells, CD34⁺ cells, and immune effector cells.

Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the composition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” refers to a diluent, adjuvant, excipient, or vehicle with which the therapeutic cells are administered. Illustrative examples of pharmaceutical carriers can be sterile liquids, such as cell culture media, water and oils, including those of petroleum, animal, vegetable or synthetic origin, such as peanut oil, soy bean oil, mineral oil, sesame oil and the like. Saline solutions and aqueous dextrose and glycerol solutions can also be employed as liquid carriers, particularly for injectable solutions. Suitable pharmaceutical excipients in particular embodiments, include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Except insofar as any conventional media or agent is incompatible with the active ingredient, its use in the therapeutic compositions is contemplated. Supplementary active ingredients can also be incorporated into the compositions.

In one embodiment, a composition comprising a pharmaceutically acceptable carrier is suitable for administration to a subject. In particular embodiments, a composition comprising a carrier is suitable for parenteral administration, e.g., intravascular (intravenous or intraarterial), intraperitoneal or intramuscular administration. In particular embodiments, a composition comprising a pharmaceutically acceptable carrier is suitable for intraventricular, intraspinal, or intrathecal administration. Pharmaceutically acceptable carriers include sterile aqueous solutions, cell culture media, or dispersions. The use of such media and agents for pharmaceutically active substances is well known in the art. Except insofar as any conventional media or agent is incompatible with the transduced cells, use thereof in the pharmaceutical compositions is contemplated.

In particular embodiments, compositions contemplated herein comprise genetically modified hematopoietic stem and/or progenitor cells or immune effector cells comprising an exogenous polynucleotide encoding a fusion polypeptide contemplated herein and a pharmaceutically acceptable carrier.

A composition comprising a cell-based composition contemplated herein can be administered by parenteral administration methods.

The pharmaceutically acceptable carrier must be of sufficiently high purity and of sufficiently low toxicity to render it suitable for administration to the human subject being treated. It further should maintain or increase the stability of the composition. The pharmaceutically acceptable carrier can be liquid or solid and is selected, with the planned manner of administration in mind, to provide for the desired bulk, consistency, etc., when combined with other components of the composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions contemplated herein include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. The term “buffer” as used herein refers to a solution or liquid whose chemical makeup neutralizes acids or bases without a significant change in pH. Examples of buffers contemplated herein include, but are not limited to, Dulbecco's phosphate buffered saline (PBS), Ringer's solution, 5% dextrose in water (D5W), normal/physiologic saline (0.9% NaCl).

The pharmaceutically acceptable carriers may be present in amounts sufficient to maintain a pH of the composition of about 7. Alternatively, the composition has a pH in a range from about 6.8 to about 7.4, e.g., 6.8, 6.9, 7.0, 7.1, 7.2, 7.3, and 7.4. In still another embodiment, the composition has a pH of about 7.4.

Compositions contemplated herein may comprise a nontoxic pharmaceutically acceptable medium. The compositions may be a suspension. The term “suspension” as used herein refers to non-adherent conditions in which cells are not attached to a solid support. For example, cells maintained as a suspension may be stirred or agitated and are not adhered to a support, such as a culture dish.

In particular embodiments, compositions contemplated herein are formulated in a suspension, where the genome edited hematopoietic stem and/or progenitor cells are dispersed within an acceptable liquid medium or solution, e.g., saline or serum-free medium, in an intravenous (IV) bag or the like. Acceptable diluents include, but are not limited to water, PlasmaLyte, Ringer's solution, isotonic sodium chloride (saline) solution, serum-free cell culture medium, and medium suitable for cryogenic storage, e.g., Cryostor® medium.

In certain embodiments, a pharmaceutically acceptable carrier is substantially free of natural proteins of human or animal origin, and suitable for storing a composition comprising a population of genome edited cells, e.g., hematopoietic stem and progenitor cells. The therapeutic composition is intended to be administered into a human patient, and thus is substantially free of cell culture components such as bovine serum albumin, horse serum, and fetal bovine serum.

In some embodiments, compositions are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better-defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions include, but are not limited to QBSF-60 (Quality Biological, Inc.), StemPro-34 (Life Technologies), and X-VIVO 10.

In a preferred embodiment, the compositions comprising genome edited hematopoietic stem and/or progenitor cells are formulated in PlasmaLyte.

In various embodiments, compositions comprising hematopoietic stem and/or progenitor cells are formulated in a cryopreservation medium. For example, cryopreservation media with cryopreservation agents may be used to maintain a high cell viability outcome post-thaw. Illustrative examples of cryopreservation media used in particular compositions include, but are not limited to, CryoStor CS10, CryoStor CS5, and CryoStor CS2.

In one embodiment, the compositions are formulated in a solution comprising 50:50 PlasmaLyte A to CryoStor CS10.

In particular embodiments, the composition is substantially free of mycoplasma, endotoxin, and microbial contamination. By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells. In particular embodiments, compositions comprising hematopoietic stem or progenitor cells transduced with a retroviral vector contemplated herein contains about 0.5 EU/mL to about 5.0 EU/mL, or about 0.5 EU/mL, 1.0 EU/mL, 1.5 EU/mL, 2.0 EU/mL, 2.5 EU/mL, 3.0 EU/mL, 3.5 EU/mL, 4.0 EU/mL, 4.5 EU/mL, or 5.0 EU/mL.

In certain embodiments, compositions and formulations suitable for the delivery of polynucleotides are contemplated including, but not limited to, one or more mRNAs encoding one or more reprogrammed nucleases, and optionally end-processing enzymes.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intraarterial, intraosseous, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22nd Edition. Edited by Loyd V. Allen Jr. Philadelphia, PA: Pharmaceutical Press; 2012, which is incorporated by reference herein, in its entirety.

### J. Site-Directed Mutagenesis Methods

The qualitative properties that define a given deletion or insertion (indel) are: (i) its length, in number of bases inserted or deleted; (ii) its longitudinal position along the chromosome, usually stated relative to the nuclease target site or breakpoint; and (iii) for insertions, the inserted sequence length and composition. Deletions are the most prominent outcomes, typically comprising 90-95% of the observed events. Their most commonly reported size characteristics tend to small (i.e., 1-20 base pairs in length, with the frequency biased toward the low end of that range) and their positional distributions have been found to be evenly distributed, covering the DNA breakpoint and emanating outward in either direction without significant bias. Exceptions to these properties are frequently hypothesized to be driven by microhomologies (small duplicated tracts of approximately 3-6 base pairs in length) positioned on either side of the DNA breakpoint. Little has been reported regarding the properties of the insertions that occur far less frequently during the application of genome editing tools. Additionally, the genotypic characteristics that relate each indel species to a phenotype (for example, how it impacts an open reading frame or whether it disrupts a transcription factor binding motif) are potentially vast and idiosyncratic to each given application.

Engineered megaTAL nucleases, and other fusion polypeptides comprising a DNA-binding domain and a homing endonuclease, have characteristics that are distinct from other gene editing platforms. For example, megaTALs are monomeric, hybrid molecules comprising a modularly assembled transcription activator-like effector (TALE) array fused to a reprogrammed homing endonuclease (HE). The TALE array, which anchors the HE at the target site, can be sized to recognize a binding site of approximately 6 to 18 base pairs. The homing endonuclease recognizes and cleaves a 22 base pair target site. The two target sites are separated by a spacer region that may be anywhere from 0 to approximately 12 base pairs in length. Two unique properties of megaTAL nucleases arise from their distinct composition: (i) the anchoring mechanism of the TALE array provides a highly biased distribution of overall binding affinity to one side of the target site; and (ii) the products of the DNA cleavage reaction catalyzed by the homing endonuclease are DNA ends with 3′ overhangs 4 base pairs in length. Conversely, ZFNs and TALENs, in which zinc finger or TALE arrays respectively are operationalized by a FokI nuclease, have a relatively even distribution of binding affinity and produce 5′ overhanging ends 4 base pairs in length. The mechanism of CRISPR DNA recognition and cleavage is fundamentally distinct in terms of DNA sequence recognition and affinity, however the products of DNA cleavage are either blunt-ended DNA ends (Cas9) or 5′ overhanging ends (Cpf1).

To evaluate the quantitative and qualitative aspects of gene editing events, the inventors of the fusion polypeptides contemplated herein, tested the co-delivery of various end-processing enzymes to characterize their impact on edited alleles. Furthermore, the inventors designed numerous multipartite megaTAL fusion proteins to evaluate how editing outcomes could be manipulated by in-line enzymatic functions.

Accordingly, the inventors surprisingly discovered that fusion polypeptides comprising a DNA-binding domain and a homing endonuclease variant, linked by a linker domain (e.g., a polypeptide linker) to an exonuclease, particularly Trex2, ExoI, or ExoX, when expressed in a cell, created elongated and directionally biased deletions, as compared to a fusion polypeptide comprising a DNA-binding domain and a homing endonuclease variant not linked to a exonuclease, i.e., when the DNA-binding domain/homing endonuclease fusion and the exonuclease were expressed separately.

Accordingly, provided are methods of site-directed mutagenesis in a cell comprising selecting a double-stranded DNA (dsDNA) target site, and introducing into the cell a fusion polypeptide, or a polynucleotide, mRNA or vector encoding a fusion polypeptide as contemplated herein, wherein the fusion peptide generates directionally biased deletions having a deletion center near the selected dsDNA target cut site in the cell.

In various embodiments, the methods comprising introducing a fusion polypeptide comprising, a DNA-binding domain and a homing endonuclease (HE) variant that binds and cleaves a selected double strand DNA (dsDNA) target site in a cell; a linker domain; and an exonuclease or biologically active fragment thereof. In some embodiments, the exonuclease is Trex2, ExoI, or ExoX. In some embodiments, the DNA-binding domain comprises a TALE DNA-binding domain (e.g., a megaTAL) or a zinc finger DNA-binding domain.

In various embodiments, the ExoX, or biologically active fragment thereof, comprises an amino acid sequence having at least 85%, 90%, 95%, 96%, 97%, 98%, or 99% identity to an amino acid sequence as set forth in SEQ ID NO: 109.

In various embodiments, the ExoI, or biologically active fragment thereof, comprises an amino acid an amino acid sequence having at least 85%, 90%, 95%, 96%, 97%, 98%, or 99% identity to an amino acid sequence as set forth in SEQ ID NO: 112.

In various embodiments, the linker domain is a peptide linker. In some embodiments, the peptide linker is a self-cleaving peptide linker. In some embodiments, the peptide linker comprises about 4 to about 30 amino acids. In some embodiments, the peptide linker is a (GGGGS) 1-4 linker (SEQ ID NOs: 117 and 150-152).

In various embodiments, the HE variant is an LAGLIDADG homing endonuclease (LHE) variant. In some embodiments, the HE variant is a variant of an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-SceI, I-ScuMI, I-SmaMI, I-SscMI, and I-Vdi141I.

In various embodiments, the contemplated methods comprise introducing a fusion polypeptide that targets a site within a immune system checkpoint gene, globin gene, gene that encodes a polypeptide that contributes to repression of γ-globin gene expression and HbF, or immunosuppressive signaling gene. In some embodiments, the target site is within a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), TCRβ, IL10Rα, IL10Rβ, TGFBR1, TGFBR2, CBL-B, PCSK9, AHR, BTK, α-globin, β-globin, γ-globin, BCL11A, KLF1, SOX6, GATA1, LSD1, alpha folate receptor (FRα), αvβ6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), melanoma antigen gene (MAGE)-A1, MAGE-A3, MAGE-A4. MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), TEM5, TEM8, trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1) gene.

In some embodiments, the target site is within a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRα), IL10Rα, TGFBR2, CBL-B, PCSK9, AHR, BTK, α-globin, β-globin, γ-globin, and BCL11A gene.

In various embodiments, the target site is within a TRAC (TCRα) gene, a CBL-B gene, or a PDCD1 (PD-1) gene. In particular embodiments, the TCRα gene target site comprises the amino acid sequence set forth in SEQ ID NO: 1. In particular embodiments, the CBL-B gene target site comprises the amino acid sequence set forth in SEQ ID NO: 2. In particular embodiments, the PD-1 gene target site comprises the amino acid sequence set forth in SEQ ID NO: 3.

As contemplated throughout the disclosure, the fusion polypeptides and related methods generate directionally biased deletions in a cell at a selected dsDNA target site. In various embodiments, the deletion center location is on the same side as the DNA-binding domain target site relative to the HE target site center location. In particular embodiments, the deletion center location is 5′ to the HE target site center location.

In various embodiments, greater than 50% of the directionally biased deletions have a deletion center location on one side of the HE target site center location. In some embodiments, greater than 51% of the directionally biased deletions have a deletion center location on one side of the HE target site center location. In some embodiments, greater than 52% of the directionally biased deletions have a deletion center location on one side of the HE target site center location. In some embodiments, greater than 53% of the directionally biased deletions have a deletion center location on one side of the HE target site center location. In some embodiments, greater than 54% of the directionally biased deletions have a deletion center location on one side of the HE target site center location. In some embodiments, greater than 55% of the directionally biased deletions have a deletion center location on one side of the HE target site center location. In some embodiments, greater than 56% of the directionally biased deletions have a deletion center location on one side of the HE target site center location. In some embodiments, greater than 57% of the directionally biased deletions have a deletion center location on one side of the HE target site center location. In some embodiments, greater than 58% of the directionally biased deletions have a deletion center location on one side of the HE target site center location. In some embodiments, greater than 59% of the directionally biased deletions have a deletion center location on one side of the HE target site center location. In some embodiments, greater than 60% of the directionally biased deletions have a deletion center location on one side of the HE target site center location. In some embodiments, greater than 65% of the directionally biased deletions have a deletion center location on one side of the HE target site center location. In some embodiments, greater than 70% of the directionally biased deletions have a deletion center location on one side of the HE target site center location. In some embodiments, greater than 75% of the directionally biased deletions have a deletion center location on one side of the HE target site center location. In some embodiments, greater than 80% of the directionally biased deletions have a deletion center location on one side of the HE target site center location.

In various embodiments, at least 50% of deletions have a deletion center greater than 4 nucleotides away from the HE target site center location. In some embodiments, at least 51% of deletions have a deletion center greater than 4 nucleotides away from the HE target site center location. In some embodiments, at least 52% of deletions have a deletion center greater than 4 nucleotides away from the HE target site center location. In some embodiments, at least 53% of deletions have a deletion center greater than 4 nucleotides away from the HE target site center location. In some embodiments, at least 54% of deletions have a deletion center greater than 4 nucleotides away from the HE target site center location. In some embodiments, at least 55% of deletions have a deletion center greater than 4 nucleotides away from the HE target site center location. In some embodiments, at least 56% of deletions have a deletion center greater than 4 nucleotides away from the HE target site center location. In some embodiments, at least 57% of deletions have a deletion center greater than 4 nucleotides away from the HE target site center location. In some embodiments, at least 58% of deletions have a deletion center greater than 4 nucleotides away from the HE target site center location. In some embodiments, at least 59% of deletions have a deletion center greater than 4 nucleotides away from the HE target site center location. In some embodiments, at least 60% of deletions have a deletion center greater than 4 nucleotides away from the HE target site center location. In some embodiments, at least 65% of deletions have a deletion center greater than 4 nucleotides away from the HE target site center location. In some embodiments, at least 70% of deletions have a deletion center greater than 4 nucleotides away from the HE target site center location. In some embodiments, at least 75% of deletions have a deletion center greater than 4 nucleotides away from the HE target site center location. In some embodiments, at least 80% of deletions have a deletion center greater than 4 nucleotides away from the HE target site center location.

In various embodiments, at least 10% of deletions have a deletion center greater than 8 nucleotides away from the HE target site center location. In some embodiments, at least 11% of deletions have a deletion center greater than 8 nucleotides away from the HE target site center location. In some embodiments, at least 12% of deletions have a deletion center greater than 8 nucleotides away from the HE target site center location. In some embodiments, at least 13% of deletions have a deletion center greater than 8 nucleotides away from the HE target site center location. In some embodiments, at least 14% of deletions have a deletion center greater than 8 nucleotides away from the HE target site center location. In some embodiments, at least 15% of deletions have a deletion center greater than 8 nucleotides away from the HE target site center location. In some embodiments, at least 16% of deletions have a deletion center greater than 8 nucleotides away from the HE target site center location. In some embodiments, at least 17% of deletions have a deletion center greater than 8 nucleotides away from the HE target site center location. In some embodiments, at least 18% of deletions have a deletion center greater than 8 nucleotides away from the HE target site center location. In some embodiments, at least 19% of deletions have a deletion center greater than 8 nucleotides away from the HE target site center location. In some embodiments, at least 20% of deletions have a deletion center greater than 8 nucleotides away from the HE target site center location. In some embodiments, at least 25% of deletions have a deletion center greater than 8 nucleotides away from the HE target site center location. In some embodiments, at least 30% of deletions have a deletion center greater than 8 nucleotides away from the HE target site center location. In some embodiments, at least 35% of deletions have a deletion center greater than 8 nucleotides away from the HE target site center location.

In various embodiments, at least 50% of deletions are 6 bps in length or greater. In some embodiments, at least 51% of deletions are 6 bps in length or greater. In some embodiments, at least 52% of deletions are 6 bps in length or greater. In some embodiments, at least 53% of deletions are 6 bps in length or greater. In some embodiments, at least 54% of deletions are 6 bps in length or greater. In some embodiments, at least 55% of deletions are 6 bps in length or greater. In some embodiments, at least 56% of deletions are 6 bps in length or greater. In some embodiments, at least 57% of deletions are 6 bps in length or greater. In some embodiments, at least 58% of deletions are 6 bps in length or greater. In some embodiments, at least 59% of deletions are 6 bps in length or greater. In some embodiments, at least 60% of deletions are 6 bps in length or greater. In some embodiments, at least 65% of deletions are 6 bps in length or greater. In some embodiments, at least 70% of deletions are 6 bps in length or greater. In some embodiments, at least 75% of deletions are 6 bps in length or greater. In some embodiments, at least 80% of deletions are 6 bps in length or greater.

In various embodiments, at least 30% of deletions are 12 bps in length or greater. In some embodiments, at least 31% of deletions are 12 bps in length or greater. In some embodiments, at least 32% of deletions are 12 bps in length or greater. In some embodiments, at least 33% of deletions are 12 bps in length or greater. In some embodiments, at least 34% of deletions are 12 bps in length or greater. In some embodiments, at least 35% of deletions are 12 bps in length or greater. In some embodiments, at least 36% of deletions are 12 bps in length or greater. In some embodiments, at least 37% of deletions are 12 bps in length or greater. In some embodiments, at least 38% of deletions are 12 bps in length or greater. In some embodiments, at least 39% of deletions are 12 bps in length or greater. In some embodiments, at least 40% of deletions are 12 bps in length or greater. In some embodiments, at least 45% of deletions are 12 bps in length or greater. In some embodiments, at least 50% of deletions are 12 bps in length or greater. In some embodiments, at least 55% of deletions are 12 bps in length or greater. In some embodiments, at least 60% of deletions are 12 bps in length or greater.

In various embodiments, the directionally biased deletions comprise a length of about 10 nucleotides. In some embodiments, the directionally biased deletions comprise a length of about 11 nucleotides. In some embodiments, the directionally biased deletions comprise a length of about 12 nucleotides. In some embodiments, the directionally biased deletions comprise a length of about 13 nucleotides. In some embodiments, the directionally biased deletions comprise a length of about 14 nucleotides. In some embodiments, the directionally biased deletions comprise a length of about 15 nucleotides. In some embodiments, the directionally biased deletions comprise a length of about 16 nucleotides. In some embodiments, the directionally biased deletions comprise a length of about 17 nucleotides. In some embodiments, the directionally biased deletions comprise a length of about 18 nucleotides. In some embodiments, the directionally biased deletions comprise a length of about 19 nucleotides. In some embodiments, the directionally biased deletions comprise a length of about 20 nucleotides.

In various embodiments, the deletion extends into the DNA-binding domain target site. In various embodiments, the deletion center location is within the DNA-binding domain target site.

In various embodiments, the method further comprises introducing into the cell an end processing enzyme, or biologically active fragment thereof, or a polynucleotide, RNA, or vector encoding an end processing enzyme (e.g., an exonuclease) in addition to the fusion polypeptide, or. In some embodiments, the end-processing enzyme, or biologically active fragment thereof, is selected from the group consisting of: Trex2, Trex1, Trex1 without transmembrane domain, Apollo, Artemis, DNA2, ExoI, ExoT, ExoIII, ExoX, Fen1, Fan1, MreII, Rad2, Rad9, TdT (terminal deoxynucleotidyl transferase), PNKP, RecE, RecJ, RecQ, Lambda exonuclease, Sox, Vaccinia DNA polymerase, exonuclease I, exonuclease III, exonuclease VII, NDK1, NDK5, NDK7, NDK8, WRN, T7-exonuclease Gene 6, avian myeloblastosis virus integration protein (IN), Bloom, Antartic Phophatase, Alkaline Phosphatase, Poly nucleotide Kinase (PNK), ApeI, Mung Bean nuclease, Hex1, TTRAP (TDP2), Sgs1, Sae2, CUP, Pol mu, Pol lambda, MUS81, EME1, EME2, SLX1, SLX4 and UL-12. In some embodiments, the end processing enzyme is an exonuclease. In particular embodiments, the exonuclease is Trex2, or biologically active fragment thereof.

In particular embodiments, provided are methods of site-directed mutagenesis in a cell comprises selecting a double-stranded DNA (dsDNA) target site, and introducing into the cell a fusion polypeptide, or a polynucleotide, mRNA or vector encoding a fusion polypeptide as contemplated herein, wherein the fusion peptide generates directionally biased deletions having a deletion center near the selected dsDNA target cut site in the cell.

In particular embodiments, the methods comprise selecting a double-stranded DNA (dsDNA) target site; introducing into a cell a fusion polypeptide comprising, a DNA-binding domain and a homing endonuclease (HE) variant that binds and cleaves a selected double strand DNA (dsDNA) target site in a cell, a linker domain, and an ExoI, or biologically active fragment thereof; and introducing a exonuclease (e.g., Trex2); wherein the method generates directionally biased deletions having a deletion center near the selected dsDNA target cut site in the cell.

In various embodiments, the method is an in vitro method. In various embodiments, the method is an ex vivo method. In various embodiments, the method is an in vivo method.

### K. Therapeutic Methods

The fusion polypeptides, genetically edited cells, compositions, and/or associated methods of gene editing contemplated herein can be used in the prevention, treatment, and amelioration of a disease or disorder, or ameliorating a disease condition or symptom associated therewith. In some embodiments, the fusion polypeptides, genetically edited cells, compositions, and/or associated methods of gene editing contemplated herein can be used in methods of treating, preventing, or inhibiting a disease (e.g., cancer, ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, hyper IGE syndrome, hemophilia) or ameliorating a disease condition or symptom associated with a disease, such as, cancer, ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, hyper IGE syndrome, hemophilia.

In some embodiments, the fusion polypeptides, genetically edited cells, compositions, and/or associated methods of gene editing contemplated herein are useful to treat, prevent, or inhibit an autosomal dominant disease, such as achondroplasia, pseudoachondroplasia, the multiple epiphyseal dysplasias, chondrodysplasias, osteogenesis imperfecta, Marfan syndrome, Polydactyly, hereditary motor sensory neuropathies I and II (Charcot-Marie-Tooth disease), myotonic dystrophy, and neurofibromatosis or ameliorate a disease condition or symptom associated with an autosomal dominant disease, such as achondroplasia, pseudoachondroplasia, the multiple epiphyseal dysplasias, chondrodysplasias, osteogenesis imperfecta, Marfan syndrome, Polydactyly, hereditary motor sensory neuropathies I and II (Charcot-Marie-Tooth disease), myotonic dystrophy, and neurofibromatosis. In some embodiments, the fusion polypeptides, genetically edited cells, compositions, and/or associated methods of gene editing contemplated herein are useful to treat, prevent, or inhibit a disease caused by misregulation of genes.

In preferred embodiments, the fusion polypeptides, genetically edited cells, compositions, and/or associated methods of gene editing contemplated herein can be used to prevent, treat, and/or ameliorate of at least one symptom associated with and immune disorder or cancer.

An “immune disorder” refers to a disease that evokes a response from the immune system. In particular embodiments, the term “immune disorder” refers to a cancer, graft-versus-host disease, an autoimmune disease, or an immunodeficiency. In one embodiment, immune disorders encompass infectious disease.

As used herein, the term “cancer” relates generally to a class of diseases or conditions in which abnormal cells divide without control and can invade nearby tissues.

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood).

As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

“Graft-versus-host disease” or “GVHD” refers complications that can occur after cell, tissue, or solid organ transplant. GVHD can occur after a stem cell or bone marrow transplant in which the transplanted donor cells attack the transplant recipient's body. Acute GVHD in humans takes place within about 60 days post-transplantation and results in damage to the skin, liver, and gut by the action of cytolytic lymphocytes. Chronic GVHD occurs later and is a systemic autoimmune disease that affects primarily the skin, resulting in the polyclonal activation of B cells and the hyperproduction of Ig and autoantibodies. Solid-organ transplant graft-versus-host disease (SOT-GVHD) occurs in two forms. The more common type is antibody mediated, wherein antibodies from a donor with blood type O attack a recipient's red blood cells in recipients with blood type A, B, or AB, leading to mild transient, hemolytic anemias. The second form of SOT-GVHD is a cellular type associated with high mortality, wherein donor-derived T cells produce an immunological attack against immunologically disparate host tissue, most often in the skin, liver, gastrointestinal tract, and bone marrow; leading to complications in these organs.

“Graft-versus-leukemia” or “GVL” refer to an immune response to a person's leukemia cells by immune cells present in a donor's transplanted tissue, such as bone marrow or peripheral blood.

An “autoimmune disease” refers to a disease in which the body produces an immunogenic (i.e., immune system) response to some constituent of its own tissue. In other words, the immune system loses its ability to recognize some tissue or system within the body as “self” and targets and attacks it as if it were foreign. Illustrative examples of autoimmune diseases include, but are not limited to: arthritis, inflammatory bowel disease, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

An “immunodeficiency” means the state of a patient whose immune system has been compromised by disease or by administration of chemicals. This condition makes the system deficient in the number and type of blood cells needed to defend against a foreign substance. Immunodeficiency conditions or diseases are known in the art and include, for example, AIDS (acquired immunodeficiency syndrome), SCID (severe combined immunodeficiency disease), selective IgA deficiency, common variable immunodeficiency, X-linked agammaglobulinemia, chronic granulomatous disease, hyper-IgM syndrome, Wiskott-Aldrich Syndrome (WAS), and diabetes.

An “infectious disease” refers to a disease that can be transmitted from person to person or from organism to organism, and is caused by a microbial or viral agent (e.g., common cold). Infectious diseases are known in the art and include, for example, hepatitis, sexually transmitted diseases (e.g., Chlamydia, gonorrhea), tuberculosis, HIV/AIDS, diphtheria, hepatitis B, hepatitis C, cholera, and influenza.

As used herein, the terms “individual” and “subject” are often used interchangeably and refer to any animal that exhibits a symptom of an immune disorder that can be treated with the fusion polypeptides, genetically edited cells, compositions, and/or associated methods of gene editing contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human subjects, are included. Typical subjects include human patients that have, have been diagnosed with, or are at risk of having an immune disorder.

As used herein, the term “patient” refers to a subject that has been diagnosed with an immune disorder that can be treated with the fusion polypeptides, genetically edited cells, compositions, and/or associated methods of gene editing contemplated elsewhere herein.

As used herein “treatment” or “treating,” includes any beneficial or desirable effect on the symptoms or pathology of a disease or pathological condition, and may include even minimal reductions in one or more measurable markers of the disease or condition being treated, e.g., cancer, GVHD, infectious disease, autoimmune disease, inflammatory disease, and immunodeficiency. Treatment can optionally involve delaying of the progression of the disease or condition. “Treatment” does not necessarily indicate complete eradication or cure of the disease or condition, or associated symptoms thereof.

As used herein, “prevent,” and similar words such as “prevention,” “prevented,” “preventing” etc., indicate an approach for preventing, inhibiting, or reducing the likelihood of the occurrence or recurrence of, a disease or condition, e.g., cancer, GVHD, infectious disease, autoimmune disease, inflammatory disease, and immunodeficiency. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated, e.g., cancer, GVHD, infectious disease, autoimmune disease, inflammatory disease, and immunodeficiency. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

In one embodiment, the fusion polypeptides, genetically edited cells, compositions, and/or associated methods of gene editing contemplated herein are used in methods of treating cancer. In particular embodiments, m the fusion polypeptides, genetically edited cells, compositions, and/or associated methods of gene editing contemplated herein are used in methods of treating solid tumors or cancers.

In particular embodiments, the fusion polypeptides, genetically edited cells, compositions, and/or associated methods of gene editing contemplated herein are used in the treatment of solid tumors or cancers including, but not limited to: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In particular embodiments, the fusion polypeptides, genetically edited cells, compositions, and/or associated methods of gene editing contemplated herein are used in the treatment of solid tumors or cancers including, without limitation, liver cancer, pancreatic cancer, lung cancer, breast cancer, bladder cancer, brain cancer, bone cancer, thyroid cancer, kidney cancer, or skin cancer.

In particular embodiments, the fusion polypeptides, genetically edited cells, compositions, and/or associated methods of gene editing contemplated herein are used in the treatment of various cancers including but not limited to pancreatic, bladder, and lung.

In particular embodiments, the fusion polypeptides, genetically edited cells, compositions, and/or associated methods of gene editing contemplated herein are used in the treatment of liquid cancers or hematological cancers.

In particular embodiments, the fusion polypeptides, genetically edited cells, compositions, and/or associated methods of gene editing contemplated herein are used in the treatment of B-cell malignancies, including but not limited to: leukemias, lymphomas, and multiple myeloma.

In particular embodiments, the fusion polypeptides, genetically edited cells, compositions, and/or associated methods of gene editing contemplated herein are used in the treatment of liquid cancers including, but not limited to leukemias, lymphomas, and multiple myelomas: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In particular embodiments, a method comprises administering a therapeutically effective amount of fusion polypeptides, composition, and/or genetically edited cells to a patient in need thereof.

In certain embodiments, the fusion polypeptides, genetically edited cells, compositions, and/or associated methods of gene editing contemplated herein are used in the treatment of patients at risk for developing a cancer. Thus, particular embodiments comprise the treatment or prevention or amelioration of at least one symptom of a cancer comprising administering to a patient in need thereof, a therapeutically effective amount of the fusion polypeptides, genetically edited cells, and/or compositions contemplated herein.

In particular embodiments, a method of treating, preventing, or ameliorating at least one symptom of a disease, or condition associated therewith is provided, comprising harvesting a population of cells from a subject; editing the population of cells according to the methods of genetic editing/mutagenesis provided herein, and administering the edited population of cells to the subject in need thereof (e.g., a subject having cancer).

The quantity and frequency of administration will be determined by such factors as the condition of the patient, and the type and severity of the patient's disease, although appropriate dosages may be determined by clinical trials.

In one embodiment, the amount of genetically edited cells, in the composition administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells.

In particular embodiments, about 1×10⁷ cells to about 1×10⁹ cells, about 2×10⁷ cells to about 0.9×10⁹ cells, about 3×10⁷ cells to about 0.8×10⁹ cells, about 4×10⁷ cells to about 0.7×10⁹ cells, about 5×10⁷ cells to about 0.6×10⁹ cells, or about 5×10⁷ cells to about 0.5×10⁹ cells are administered to a subject.

In one embodiment, the amount of genetically edited cells in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In particular embodiments, about 1×10⁶ cells/kg of bodyweight to about 1×10⁸ cells/kg of bodyweight, about 2×10⁶ cells/kg of bodyweight to about 0.9×10⁸ cells/kg of bodyweight, about 3×10⁶ cells/kg of bodyweight to about 0.8×10⁸ cells/kg of bodyweight, about 4×10⁶ cells/kg of bodyweight to about 0.7×10⁸ cells/kg of bodyweight, about 5×10⁶ cells/kg of bodyweight to about 0.6×10⁸ cells/kg of bodyweight, or about 5×10⁶ cells/kg of bodyweight to about 0.5×10⁸ cells/kg of bodyweight are administered to a subject.

One of ordinary skill in the art would recognize that multiple administrations of the compositions contemplated in particular embodiments may be required to affect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

The administration of the compositions contemplated in particular embodiments may be carried out in any convenient manner, including by aerosol inhalation, injection, ingestion, transfusion, implantation or transplantation. In a preferred embodiment, compositions are administered nasally, orally, enterally, or parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Reproducibility of Indel Length and Distribution Induced by MegaTALs

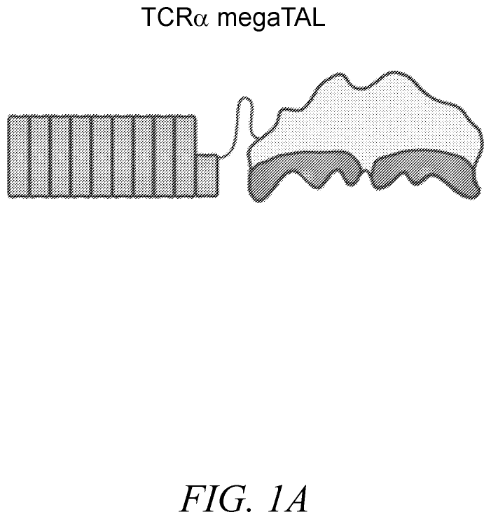

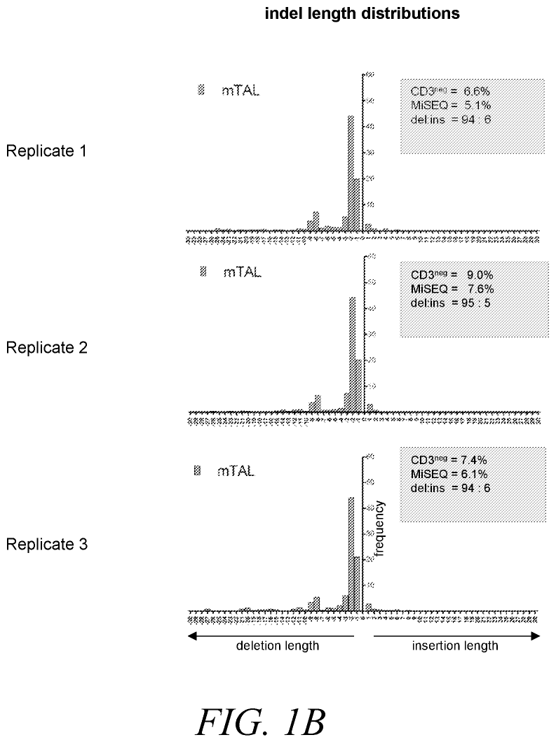

Gene editing at the TRAC locus (SEQ ID NO: 1) was used as a model system to evaluate the properties of indel events generated by megaTAL nucleases. A low/moderate efficiency megaTAL (for example SEQ ID NOs: 5-7) was used for these studies to provide dynamic range for any downstream manipulations that were hypothesized to alter editing rates. Several independent experiments were performed using primary human T cells activated and expanded from distinct PBMC donors. Following PBMC thawing, activation and culture for a period of 3-4 days, in vitro transcribed, capped and poly-adenylated mRNA encoding a megaTAL targeting exon 1 of the TRAC locus (TCRα megaTAL) were electroporated. The resulting cells were cultured for an additional outgrowth period of 7-10 days to allow for dilution and degradation of the delivered mRNA and completion of the editing process. Polymerase chain reaction (PCR) was used to amplify the TRAC locus and subsequent deep sequencing of the PCR amplicon enabled the characterization of the bulk editing events caused by the megaTAL. Frequency histograms tallying the sizes of the editing events are shown in FIG. 1B. The most frequently observed indel lengths were 2 base pairs long (cumulatively representing ˜40-45% of the total population of events), while 1 base pair deletions (˜20%) and 3, 8, and 9 base pair deletions (5-10% each) were also observed at a high frequency. The relative frequencies of these indel size populations were highly reproducible across technical replicates and independent experiments, illustrating the consistency of the indel length distributions during megaTAL gene editing.

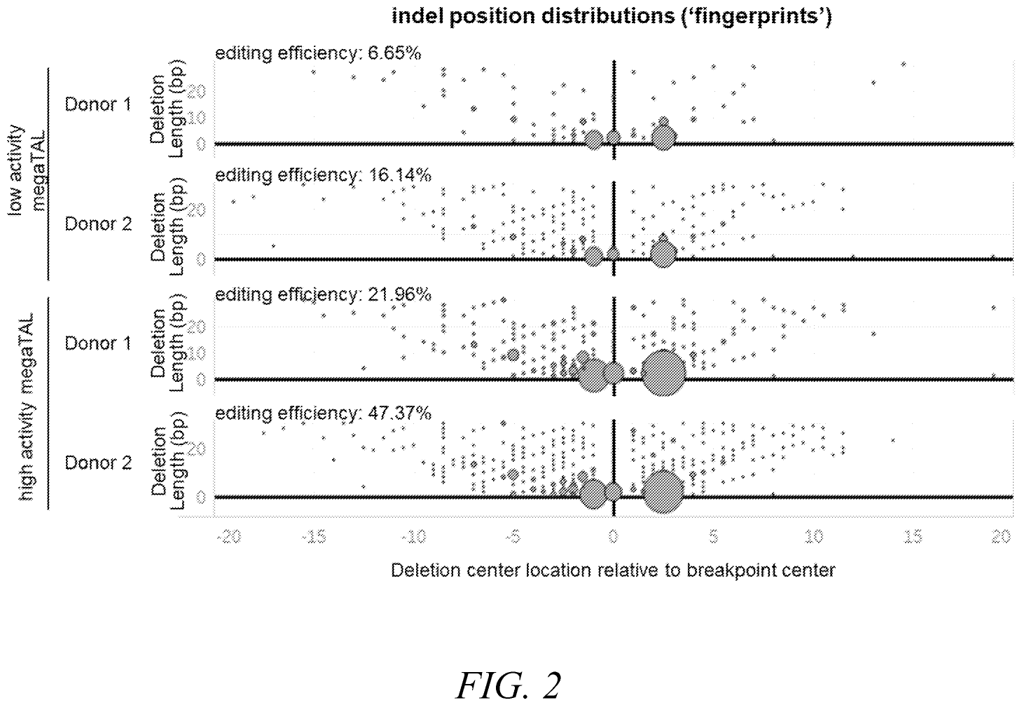

Each bar in the histograms shown in FIG. 1B represents a population of edited alleles with the same indel length but potentially different positions relative to the megaTAL target site. FIG. 2 shows each unique edited species plotted according to its positional location relative to the megaTAL target site breakpoint center. These “fingerprints” plot each edited species according to its length (y-axis), position (x-axis), and frequency (circle size), thus capturing both quantitative and qualitative properties of each edited species. The position of a deletion was calculated as the location of its midpoint 5′ (negative) or 3′ (positive) to the center of the 22 base pair homing endonuclease target site. In this way, the distribution of deletion species relative to the breakpoint center could be monitored for changes in both frequency and location. Independent experiments using distinct PBMC donors, as well as using TCRα megaTAL constructs with either low (for example SEQ ID NOs: 5-7) or high (SEQ ID NOs: 8-10) editing efficiencies, illustrate that individual deletion species—each recurring deletion event of a specific composition-occurs at a reproducible frequency. Thus, the overall megaTAL deletion species population has relative qualitative and quantitative properties that are both highly consistent and independent of the enzymatic rate of the endonuclease reaction.

### Example 2

### Trex2 Qualitatively Alters MegaTAL Edited Alleles

Co-delivery of Three Prime Repair Exonuclease-2 (Trex2) with gene editing nucleases has previously been shown to enhance gene editing efficiency, particularly with megaTALs owing to their 3′ overhang endonuclease reaction products. However, the qualitative impact of Trex2 co-delivery on editing outcomes have not been thoroughly assessed. Therefore, evaluation of the indel properties of two distinct modes of co-delivery was performed; direct fusion of Trex2 to the C-terminus of a megaTAL; and co-expression of Trex2 and a megaTAL as independent polypeptides.

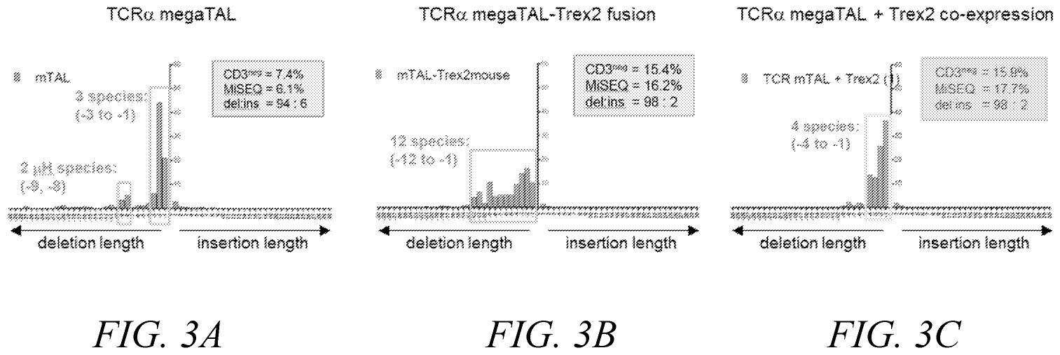

Activated primary human T cell samples were electroporated with in vitro transcribed, capped and poly-adenylated mRNA encoding the TCRα megaTAL (FIG. 3A), mRNA encoding TCRα megaTAL-Trex2 fusion (FIG. 3B), or two distinct mRNAs, one encoding TCRα megaTAL and the other encoding Trex2 (FIG. 3C). The resulting cells were cultured for an additional outgrowth period of 7-10 days to allow for dilution and degradation of the delivered mRNA and completion of the editing process. Polymerase chain reaction (PCR) was used to amplify the TRAC locus and subsequent deep sequencing of the PCR amplicon enabled the characterization of the bulk editing events caused by the megaTAL. Frequency histograms tallying the sizes of the editing events are shown in FIGS. 3A-3C. Direct fusion of the Trex2 exonuclease to the TCRα megaTAL (for example SEQ ID NOs: 11-13) resulted in a change in the deletion length distribution, with deletions up to 12 base pairs in length comprising a significant percentage of the indel species. Independent co-expression of the TCRα megaTAL and the Trex2 exonuclease (for example SEQ ID NOs: 14-16) resulted in a narrowing of the indel species toward deletions 1 to 4 base pairs in length, consistent with a model of distributive exonuclease activity with a preference for single-stranded 3′ overhangs. In each Trex2 delivery scenario, the overall editing rate and the ratio of deletions to insertions increased relative to the delivery of the TCRα megaTAL alone.

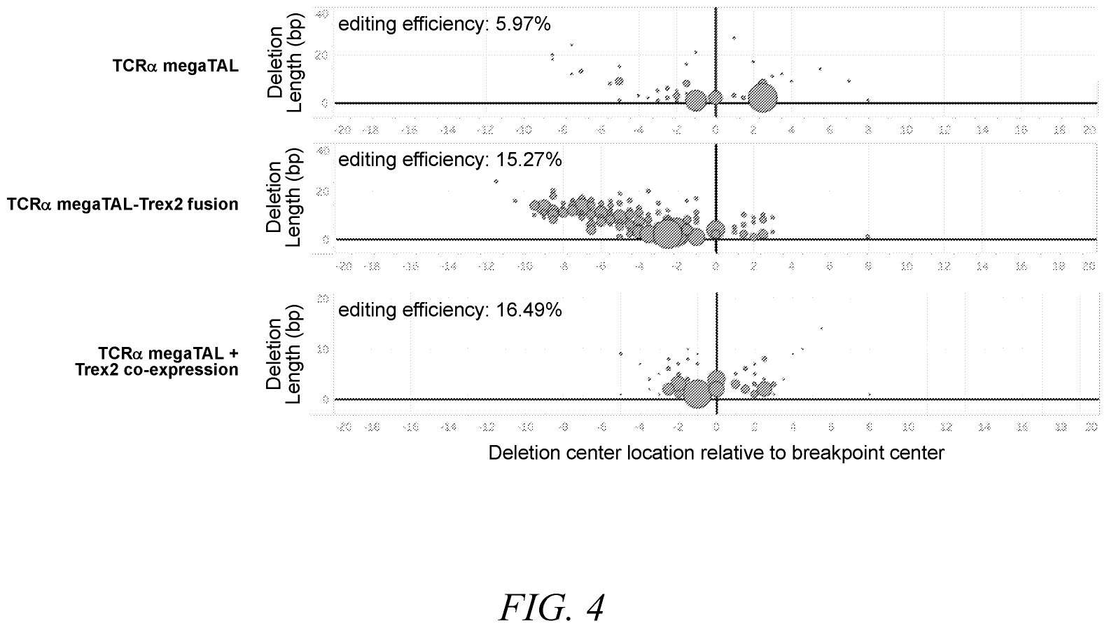

FIG. 4 shows the deletion fingerprint plots for each sample in FIGS. 3A-3C, capturing both quantitative and qualitative properties of each edited species. As in FIG. 2, the position of a deletion was calculated as the location of its midpoint 5′ (negative) or 3′ (positive) to the center of the 22 base pair homing endonuclease target site. The distribution of the deletion species relative to the breakpoint center showed marked differences between the TCRα megaTAL, TCRα megaTAL-Trex2 fusion, and TCRα megaTAL plus Trex2 co-delivery editing outcomes (see Tables 3-5). In the megaTAL-Trex2 fusion, emergence of deletion species whose positions were skewed heavily in the 5″ direction was observed. This pattern implies that there is preferential exonuclease activity occurring at the DNA breakpoint end that remains in cis with, or on the same side of the breakpoint as, the TALE array.

Additionally, we observed a steep drop-off in deletion length and position that corresponds with the 3′ edge of the TALE array binding site. Notwithstanding, we surprisingly observed deletion lengths that extended into the TALE binding site. This further implies that there is a mutually exclusive relationship between exonuclease activity and the presence of the TALE at its binding site, further solidifying the TALE array's role in positioning Trex2 to perform its exonuclease function. Deletion centers, however, stayed outside the TALE binding site. Conversely, Trex2 co-expression as a separate polypeptide did not cause any substantial directional skewing, despite altering the species composition toward a population of deletions with sizes in the 1-4 bp range centered on the DNA breakpoint.

TABLE 3

| | % edited species | | % edited species on |
|---|---|---|---|
| | on the TAL side | | the non-TAL side |
| | and more than 2 bp | % edited species | and more than 2 bp |
| | from breakpoint | centered (±2 bp of | from breakpoint |
| Polypeptide | center | breakpoint center) | center |
| TCRa megaTAL | 17.5% | 35.01% | 45.24% |
| TCRa megaTAL-Trex2 | 62.54% | 27.24% | 2.42% |
| fusion | | | |
| TCRa megaTAL + Trex2 | 10.80% | 77.40% | 12.22% |
| co-expression | | | |

TABLE 4

| | % deletions greater | % deletions greater |
|---|---|---|
| Polypeptide | than 6 bp | than 12 bp |
| TCRa megaTAL | 17.97% | 7.17% |
| TCRa megaTAL-Trex2 | 35.49% | 14.21% |
| fusion | | |
| TCRa megaTAL + Trex2 | 4.66% | 0.83% |
| co-expression | | |

TABLE 5

| | % deletion centers | % deletion centers |
|---|---|---|
| | greater than 4 bp from | greater than 8 bp from |
| Polypeptide | breakpoint center | breakpoint center |
| TCRa megaTAL | 13.22% | 6.90% |
| TCRa megaTAL-Trex2 | 31.83% | 14.21% |
| fusion | | |
| TCRa megaTAL + Trex2 | 3.51% | 2.82% |
| co-expression | | |

### Example 3

### Assessment of Trex2 Homolog Edited Alleles

To confirm that the enzymatic activity of Trex2 was directly responsible for the observed changes in the editing outcomes, versus potentially confounding interplay with the DNA repair machinery, several Trex2 homolog ORFs were fused to the TCRα megaTAL (for example SEQ ID NOs: 17-34). Activated primary human T cell samples were electroporated with in vitro transcribed, capped and polyadenylated mRNA encoding these fusion proteins, then monitored target site editing by flow cytometric analysis of expression of the TCR complex and by characterizing indel properties.

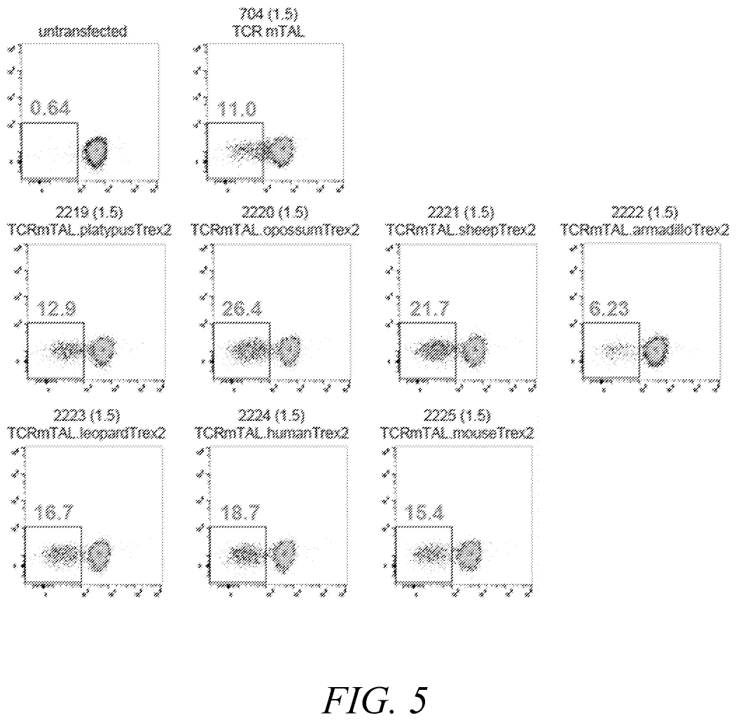

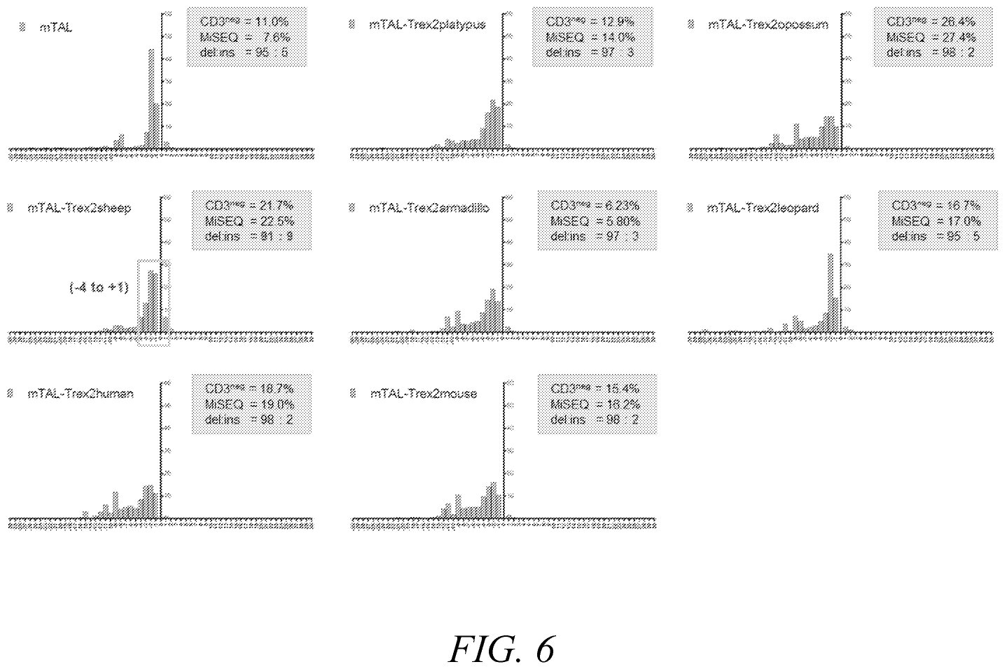

FIG. 5 shows that many of the Trex2 homolog fusion proteins enhanced the overall editing to a similar extent as the human Trex2 ORF. Flow cytometry staining for the CD3 components that combine with the TCR alpha and beta chains on the cell surface was used to assess editing efficiency. The frequency of cells that have lost CD3 staining therefore correlates with the rate of editing at the TCRα locus. Analysis of the distribution of indel species, as shown in FIG. 6, further confirms that the platypus, opossum, armadillo, and mouse Trex2 proteins cause editing outcomes with properties consistent with those caused by human Trex2 ORF when fused to the TCRα megaTAL. Of note, sheep Trex2 caused a unique indel spectrum enriched for 1-4 bp deletions and, surprisingly, a high frequency of 1 bp insertions.

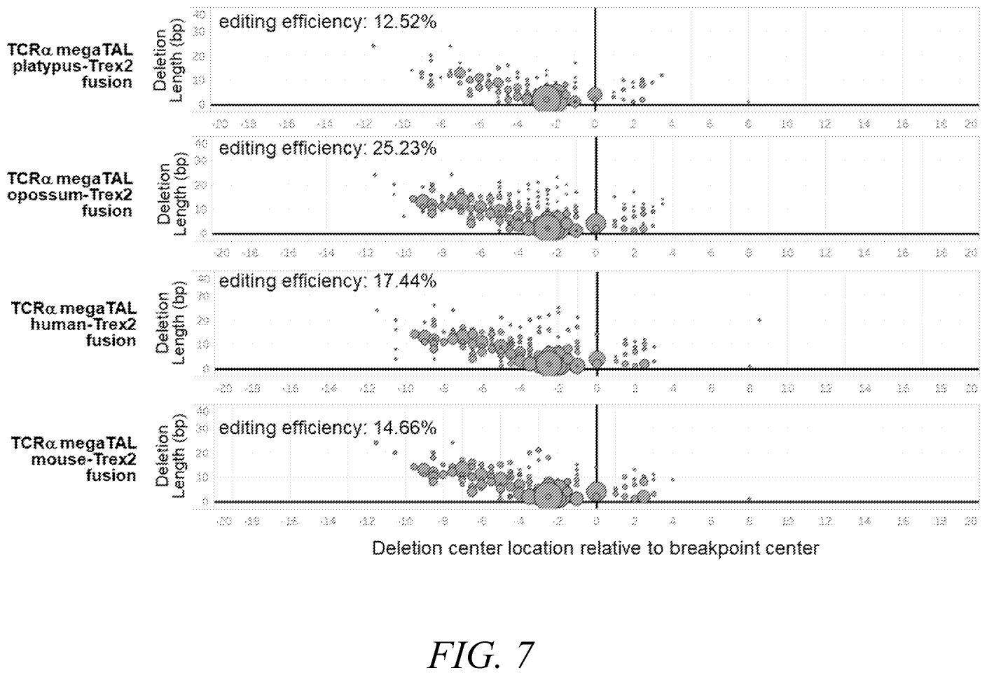

The deletion properties of the TCRα megaTAL-Trex2 homolog fusion proteins, as shown in the fingerprint plots displayed in FIG. 7, mirror those observed for the human Trex2 protein. Without wishing to be bound by any particular theory, this data suggests that Trex2 exonuclease activity is the primary determinant of the observed deletion properties. The extended deletion length outcomes are thus likely to be independent of interaction with other DNA repair machinery. The increased occurrence of insertions observed for the sheep Trex2 homolog indicates that this variant may have a unique enzymatic mechanism, such as potentially a residual template independent polymerase activity. Finally, the highly consistent and sharp demarcations in deletion size and position further suggest that the directional excision of bases may be limited by the association and dissociation kinetics and/or steric properties of the TALE array's interaction with its binding site.

### Example 4

### Identification of Exonucleases that Uniquely Impact MegaTAL Editing

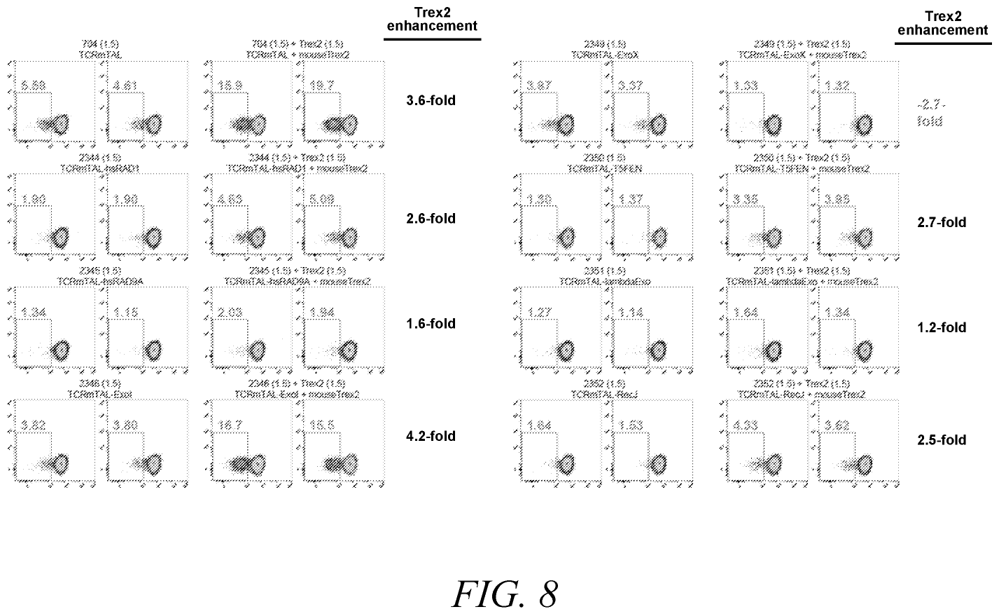

To determine whether other exonucleases having distinct substrate specificities and/or processivity properties could uniquely alter gene editing outcomes, select exonucleases or exonuclease domain ORFs were fused to the TCRα megaTAL (for example SEQ ID NOs: 35-55) and their impact on gene editing outcomes was examined. Editing rates both as stand-alone TCRα megaTAL fusion proteins as well as during co-expression with Trex2 were examined. The rationale for testing in both scenarios was two-fold: (i) to allow Trex2 activity to produce distinct and potentially preferential substrates for the test exonucleases; and (ii) to look for a loss in Trex2 mediated editing rate enhancement as a proxy for ends being meaningfully processed by the test exonuclease. FIG. 8 depicts the results of the exonuclease screen, with each sample run in duplicate and tested in the absence (left) or presence (right) of Trex2 co-expression.

Of the exonucleases tested, most did not distinctly alter editing rates or editing rate enhancement by Trex2 (measured as a ratio of CD3 negative cells in the Trex2 sample over delivery of the megaTAL-exonuclease fusion alone). Several reduced overall editing significantly, indicating that they may have significantly affected protein stability and/or expression. Fusion of the TCRα megaTAL to ExoI resulted in a modest increase in the Trex2 enhancement ratio, suggesting that perhaps these two exonucleases were synergistically modifying DNA break processing events. Notably, fusion of the TCRα megaTAL to ExoX resulted in a complete loss of Trex2 enhancement.

### Example 5

### Assessment of MegaTAL-ExoI and MegaTAL-ExoX Editing Profiles

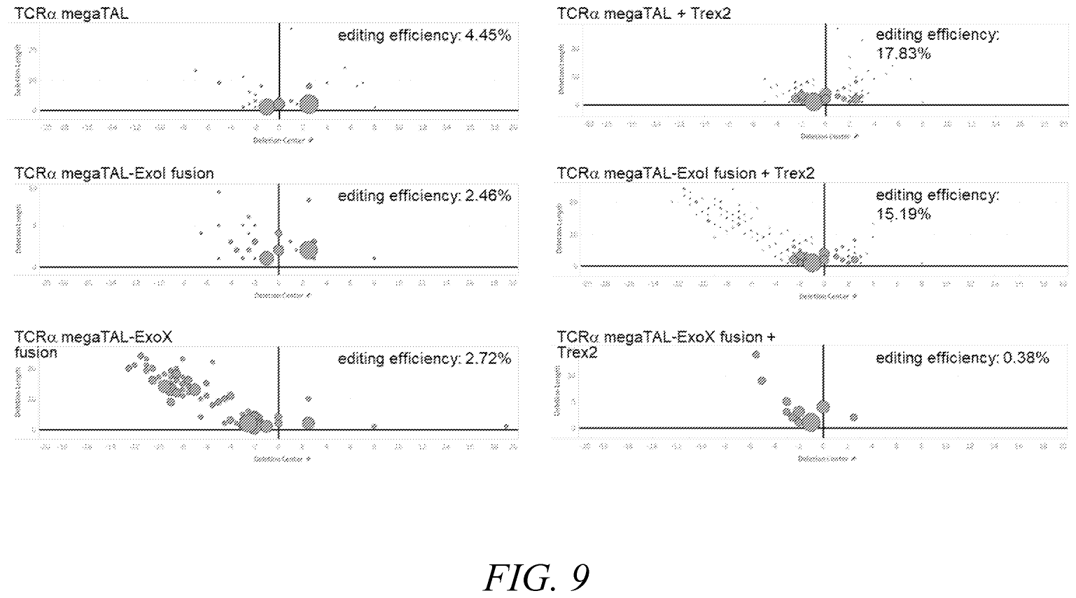

To further understand the qualitative aspects of the gene editing events described in FIG. 8, amplicon sequencing and deletion fingerprint analysis was performed as previously described. Those exonucleases that did not impact on the pattern of TCRα megaTAL editing rates, also failed to show fingerprint changes (data not shown). However, FIG. 9 shows that while fusion of the TCRα megaTAL to ExoI did not alter the deletion profile relative to the TCRα megaTAL alone, when Trex2 was co-expressed, a series of long, directionally-biased deletion events emerged.

The megaTAL-ExoI fusion generates deletion events that are biased in the direction of the TALE array. In contrast with the sharply demarcated megaTAL-Trex2 fusion deletion events, the megaTAL-ExoI deletion events appeared to be of greater length and thus extend beyond the TALE array binding site.

Fusion of the TCRα megaTAL to ExoX resulted in a deletion profile distinct from that observed with the megaTAL-ExoI fusion. In the absence of Trex2, the TCRα megaTAL-ExoX fusion generates long, directionally-biased deletions with high efficiency. Unlike megaTAL-Trex2 fusion proteins, the deletion centers observed were greater such that they did progress beyond the TALE array binding site at an appreciable frequency. It is possible that the megaTAL-ExoX fusion has a unique exonuclease mechanism that leads to longer deletions with deletion centers further away from the cut site relative to megaTAL-Trex2 fusion proteins.

In FIG. 9, when Trex2 is co-expressed with the TCRα megaTAL-ExoX fusion, editing events became almost undetectably rare and depleted of the long, directionally biased deletions that were apparent with the megaTAL-ExoX fusion.

### Example 6

### Further Assessment of MegaTAL-ExoX Fusion Protein Editing Profiles

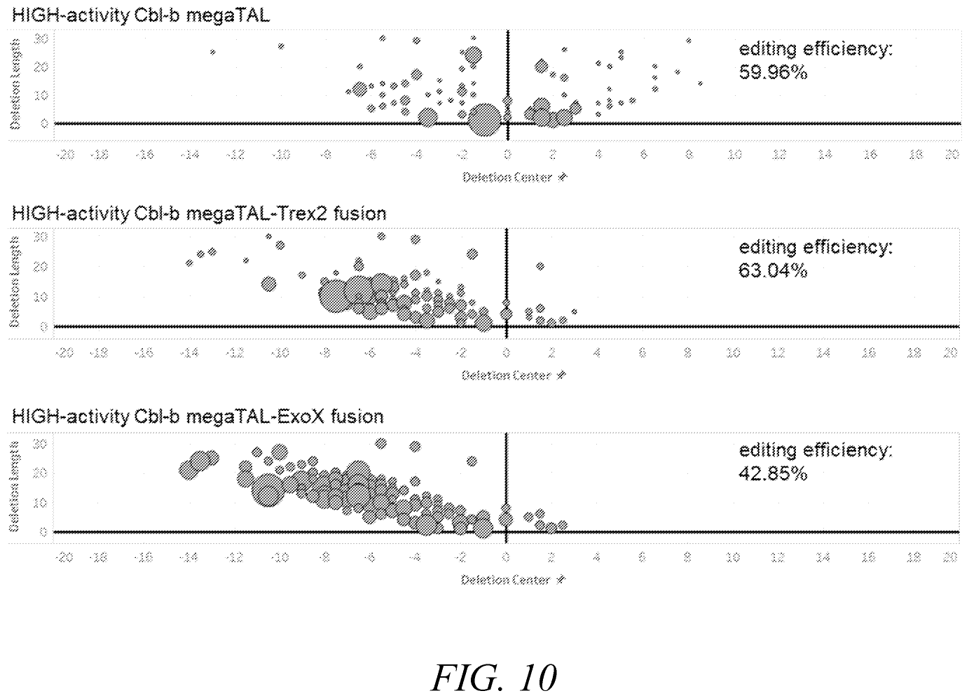

Given the observed pattern of deletions that emerged when the TCRα megaTAL-ExoX fusion evaluated, several additional megaTAL-ExoX fusion proteins targeting different genetic loci were tested. Activated primary human T cell samples were electroporated with in vitro transcribed, capped and poly-adenylated mRNA encoding either a high-activity CBL-B targeting megaTAL, a high-activity CBL-B megaTAL-Trex2 fusion, or a high-activity CBL-B megaTAL-ExoX fusion (for example SEQ ID NOs: 56-64). The resulting cells were cultured for an additional outgrowth period of 7-10 days to allow for dilution and degradation of the delivered mRNA and completion of the editing process. Polymerase chain reaction (PCR) was used to amplify the CBL-B locus and subsequent deep sequencing of the PCR amplicon enabled characterization of the editing rates and indel properties proximal to the CBL-B target site (SEQ ID NO: 3). FIG. 10 shows the deletion fingerprints observed for each of these three high-activity CBL-B megaTAL formats (see also Tables 6-8). The high-activity CBL-B megaTAL generates a distribution of deletion species similar to the TCRα megaTAL, with the most frequent events being 1-5 bp deletions, and no significant directional biases. Likewise, the high-activity CBL-B megaTAL-Trex2 fusion lengthened the deletions species and skewed them heavily in the 5′ direction, confirming for an orthogonal megaTAL target the preferential exonuclease activity occurring at the DNA breakpoint end that remains in cis with, or on the same side of the breakpoint as, the TALE array. Also recurring in the high-activity CBL-B megaTAL-Trex2 fusion was the steep drop-off in deletion length and position that corresponds with the 3′ edge of the TALE array binding site.

TABLE 6

| | % edited species | | % edited species on |
|---|---|---|---|
| | on the TAL side | | the non-TAL side |
| | and more than 2 bp | % edited species | and more than 2 bp |
| | from breakpoint | centered (±2 bp of | from breakpoint |
| Polypeptide | center | breakpoint center) | center |
| HIGH-activity CBL-B | 28.57% | 45.13% | 18.13% |
| megaTAL | | | |
| HIGH-activity CBL-B | 74.98% | 14.96% | 3.16% |
| megaTAL-Trex2 fusion | | | |
| HIGH-activity CBL-B | 78.41% | 11.58% | 2.29% |
| megaTAL-ExoX fusion | | | |

TABLE 7

| | % deletions greater | % deletions greater |
|---|---|---|
| Polypeptide | than 6 bp | than 12 bp |
| HIGH-activity CBL-B | 46.15% | 33.74% |
| megaTAL | | |
| HIGH-activity CBL-B | 71.70% | 32.60% |
| megaTAL-Trex2 fusion | | |
| HIGH-activity CBL-B | 77.83% | 56.90% |
| megaTAL-ExoX fusion | | |

TABLE 8

| | % deletion centers | % deletion centers |
|---|---|---|
| | greater than 4 bp from | greater than 8 bp from |
| Polypeptide | breakpoint center | breakpoint center |
| HIGH-activity CBL-B | 18.16% | 5.47% |
| megaTAL | | |
| HIGH-activity CBL-B | 60.58% | 8.22% |
| megaTAL-Trex2 fusion | | |
| HIGH-activity CBL-B | 67.23% | 28.19% |
| megaTAL-ExoX fusion | | |

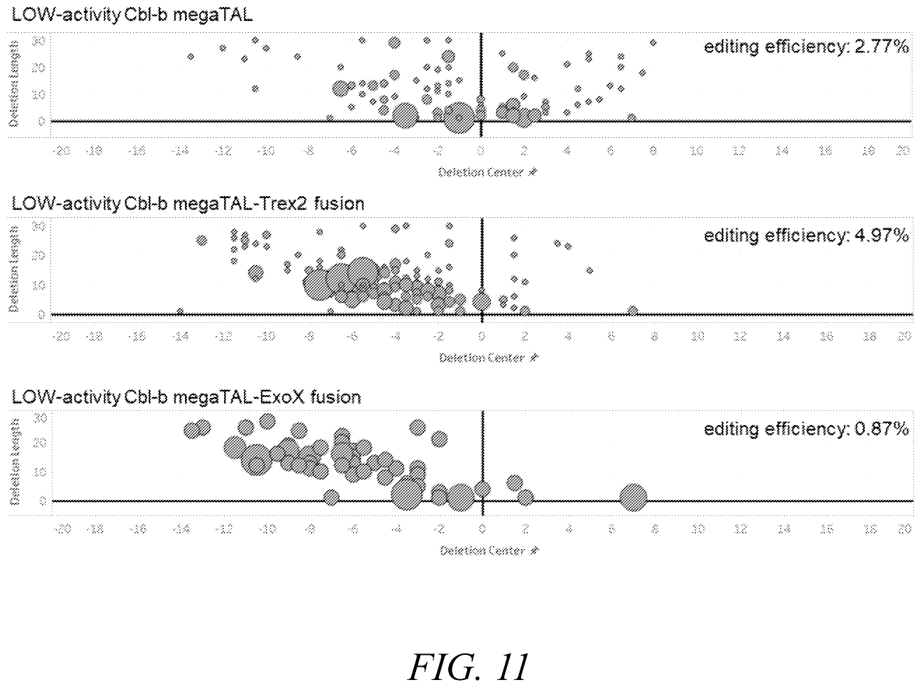

FIG. 11 shows representative deletion fingerprints from T cells treated with a similar series of CBL-B megaTAL, megaTAL-Trex2, and megaTAL-ExoX fusion proteins, however these employ a several-fold lower activity megaTAL (for example SEQ ID NOs: 65-73). See also Tables 9-11. There is a substantial similarity in the spectrum of deletion species observed when comparing between these low-activity CBL-B megaTALs and megaTAL fusion proteins to their high-activity counterparts displayed in FIG. 10. This illustrates that the unique deletion outcomes observed when these two exonucleases are fused to a megaTAL are independent of the enzymatic rate of the endonuclease reaction. These results indicate both a highly efficient exonuclease processing rate per endonuclease generated break, and furthermore illustrates that Trex2 and ExoX are distinctly impacting the gene editing outcomes through qualitatively different mechanisms and not simply identical mechanisms happening with different efficiencies.

TABLE 9

| | % edited species | | % edited species on |
|---|---|---|---|
| | on the TAL side | | the non-TAL side |
| | and more than 2 bp | % edited species | and more than 2 bp |
| | from breakpoint | centered (±2 bp of | from breakpoint |
| Polypeptide | center | breakpoint center) | center |
| LOW-activity CBL-B | 31.05% | 39.71% | 11.91% |
| megaTAL | | | |
| LOW-activity CBL-B | 72.84% | 14.29% | 1.81% |
| megaTAL-Trex2 fusion | | | |
| LOW-activity CBL-B | 58.62% | 10.34% | 5.75% |
| megaTAL-ExoX fusion | | | |

TABLE 10

| | % deletions greater | % deletions greater |
|---|---|---|
| Polypeptide | than 6 bp | than 12 bp |
| LOW-activity CBL-B | 36.82% | 26.71% |
| megaTAL | | |
| LOW-activity CBL-B | 68.01% | 30.58% |
| megaTAL-Trex2 fusion | | |
| LOW-activity CBL-B | 50.57% | 37.93% |
| megaTAL-ExoX fusion | | |

TABLE 11

| | % deletion centers | % deletion centers |
|---|---|---|
| | greater than 4 bp from | greater than 8 bp from |
| Polypeptide | breakpoint center | breakpoint center |
| LOW-activity CBL-B | 17.33% | 5.05% |
| megaTAL | | |
| LOW-activity CBL-B | 57.95% | 6.24% |
| megaTAL-Trex2 fusion | | |
| LOW-activity CBL-B | 47.12% | 22.99% |
| megaTAL-ExoX fusion | | |

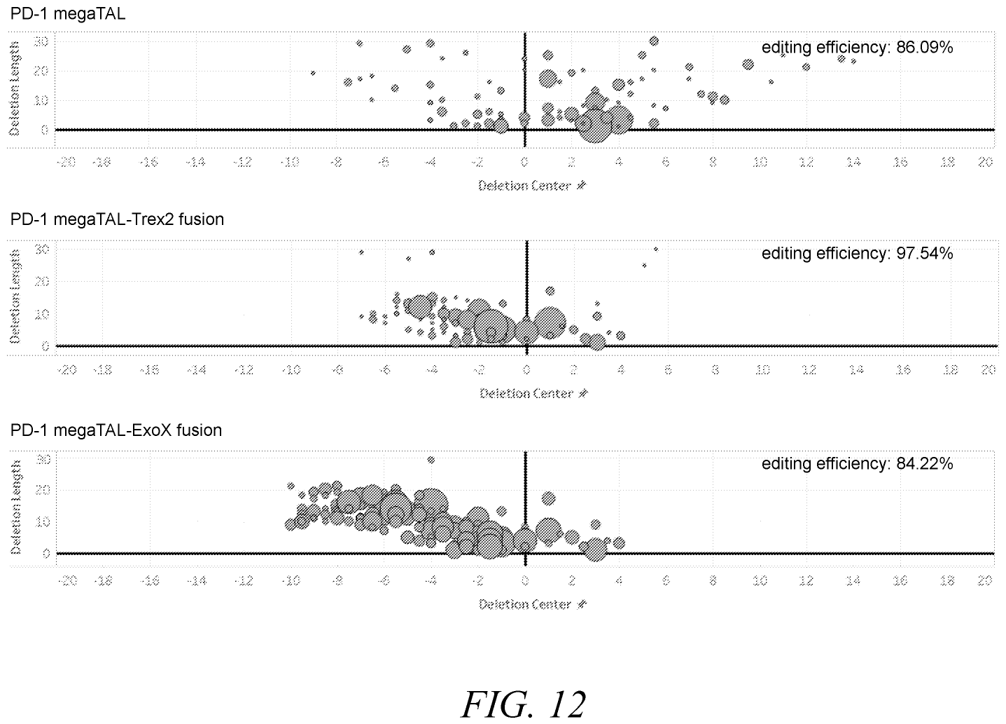

A third series of enzymes was constructed targeting the PD-1 locus. FIG. 12 shows representative deletion fingerprints from T cells electroporated mRNA encoding either a PD-1 megaTAL, PD-1 megaTAL-Trex2 fusion, or a PD-1 megaTAL-ExoX fusion (for example SEQ ID NOs: 74-82). See also Tables 12-14. As described for the TCRα and CBL-B megaTALs and exonuclease fusion proteins, the deletion species observed for each of the three PD-1 megaTAL samples followed the consistent trend of having non-directional, small deletion species in the stand-alone PD-1 megaTAL, 5′ directional and longer species emerged when that target was exposed to the PD-1 megaTAL-Trex2 fusion protein, and longer still were the species arising from treatment with PD-1 megaTAL-ExoX fusion protein. The persistence of these observed patterns across several megaTALs tested illustrates the robustness of the distinct editing outcomes that occur upon megaTAL fusion to different exonuclease domains.

TABLE 12

| | % edited species | | % edited species on |
|---|---|---|---|
| | on the TAL side | | the non-TAL side |
| | and more than 2 bp | % edited species | and more than 2 bp |
| | from breakpoint | centered (±2 bp of | from breakpoint |
| Polypeptide | center | breakpoint center) | center |
| PD-1 megaTAL | 22.13% | 22.91% | 52.62% |
| PD-1 megaTAL | 31.95% | 53.89% | 9.03% |
| megaTAL-Trex2 fusion | | | |
| PD-1 megaTAL | 62.09% | 27.27% | 4.86% |
| megaTAL-ExoX fusion | | | |

TABLE 13

| | % deletions greater | % deletions greater |
|---|---|---|
| Polypeptide | than 6 bp | than 12 bp |
| PD-1 megaTAL | 52.42% | 40.30% |
| PD-1 megaTAL | 49.63% | 13.17% |
| megaTAL-Trex2 fusion | | |
| PD-1 megaTAL | 63.87% | 35.47% |
| megaTAL-ExoX fusion | | |

TABLE 14

| | % deletion centers | % deletion centers |
|---|---|---|
| | greater than 4 bp from | greater than 8 bp from |
| Polypeptide | breakpoint center | breakpoint center |
| PD-1 megaTAL | 15.48% | 9.33% |
| PD-1 megaTAL | 14.75% | 1.58% |
| megaTAL-Trex2 fusion | | |
| PD-1 megaTAL | 43.86% | 9.83% |
| megaTAL-ExoX fusion | | |

### Example 7

### Assessment of PD-1 MegaTAL-ExoX Fusion Protein Editing Profiles

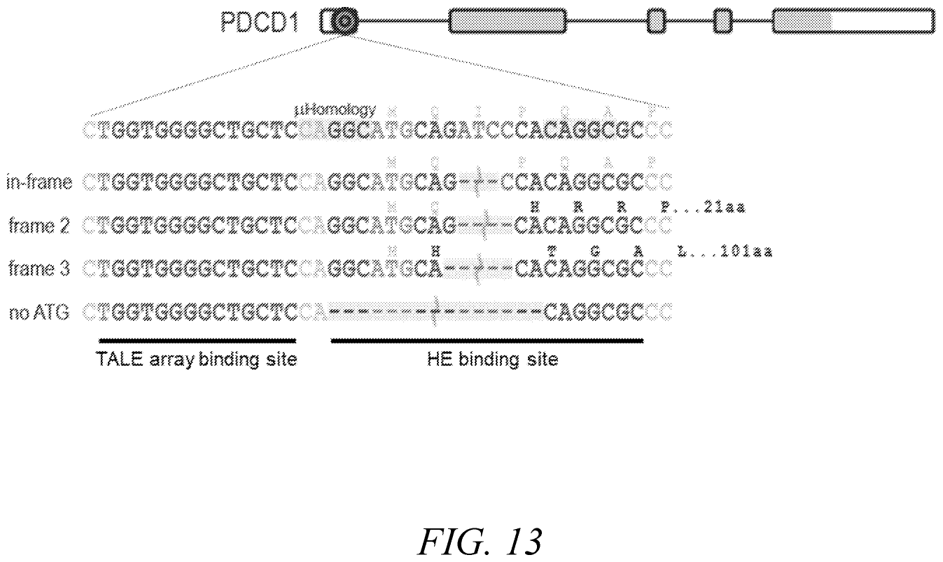

FIG. 13 depicts the PD-1 megaTAL target site, which lies proximal to the ATG start codon of the PDCD1 gene. The 22 bp PD-1 HE target site (SEQ ID NO: 2) is approximately centered on the third codon encoding an isoleucine residue that is part of the signal sequence that targets PD-1 for expression on the plasma membrane. The TALE array binding site (SEQ ID NO: 4) lies 5′ of the ATG start codon. This target site orientation raises the potential for distinct megaTAL or megaTAL-exonuclease fusion compositions to differentially impact expression of PD-1 depending on their respective distributions of deletion species.

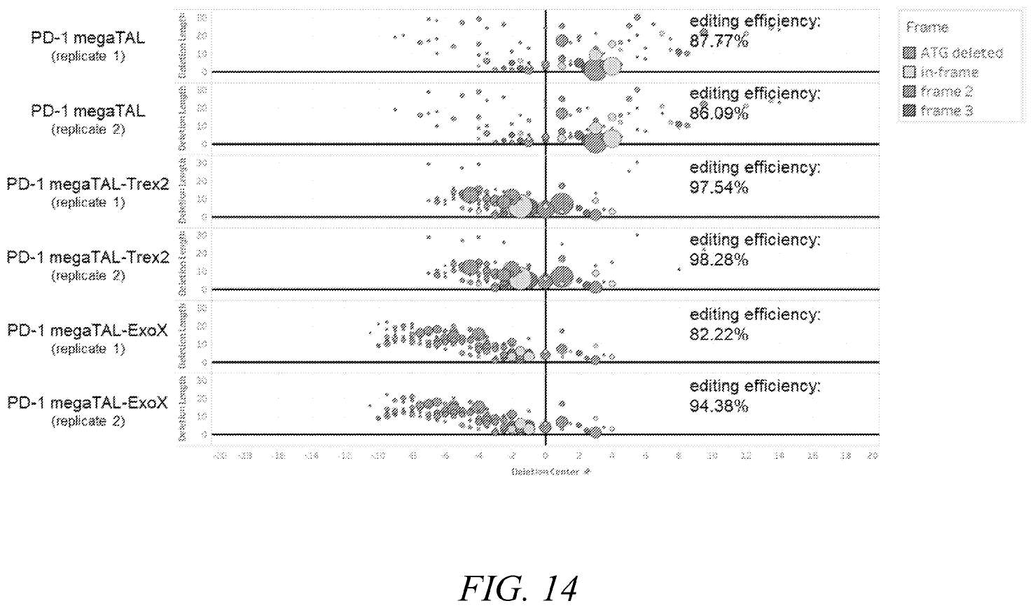

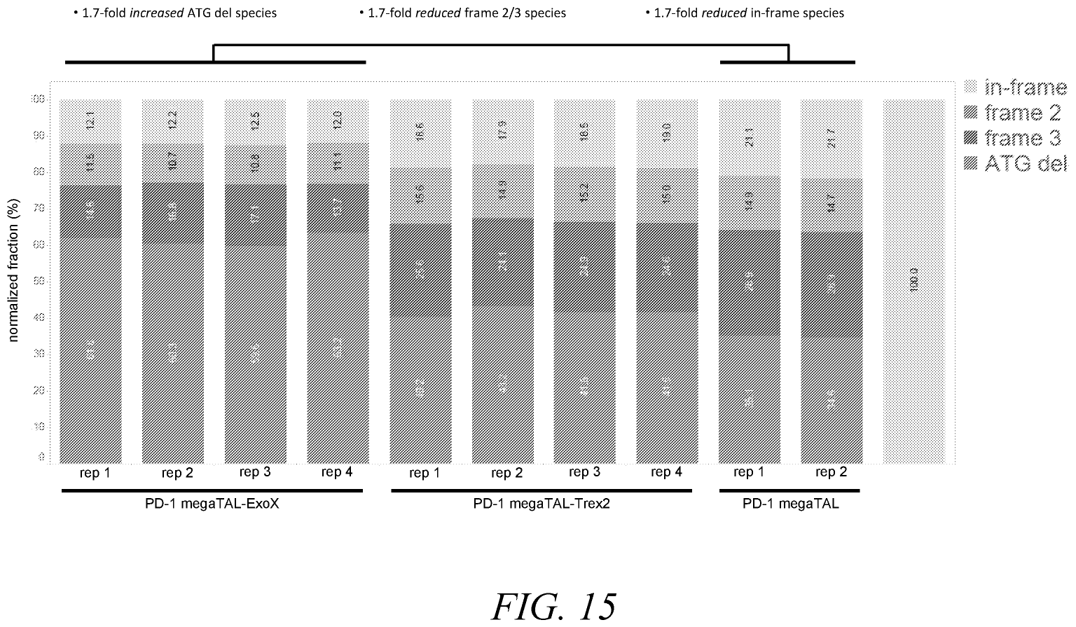

Categories of deletion species were assigned according to their impact on the PD-1 ORF: ATG-deleted; in-frame; frame 2; or frame 3; an example deletion type for each is shown in FIG. 13. For the PD-1 megaTAL and the megaTAL-exonuclease fusion protein samples described in FIG. 12, the frame characteristics of each deletion species were bioinformatically analyzed. FIG. 14 shows the outcome of this analysis by overlaying the frame categories of the gene editing species onto deletion fingerprint plots. Since the PD-1 megaTAL creates a broad spectrum of deletion species, the frame characteristics are somewhat evenly distributed across the deletion fingerprint analysis, with ATG-deleted species occupying the upper levels of the plot where long deletions are represented. Conversely, the PD-1 megaTAL-Trex2 fusion protein causes a shift in the locations of the in-frame, frame 2, and frame 3 species. When the normalized fraction of each category of species are compared, as shown in FIG. 15, the PD-1 megaTAL and the PD-1 megaTAL-Trex2 fusion do not significantly differ. This is because the occurrence of long, directional deletions that reach through the ATG start codon, while increased, have effectively replaced the smattering of long (but non-directional) deletions that occur with the PD-1 megaTAL.

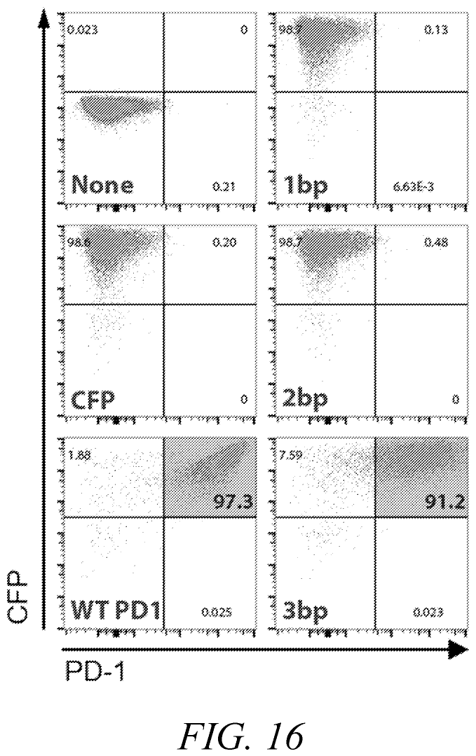

Conversely, the increased deletion lengths caused by the PD-1 megaTAL-ExoX fusion protein substantially increases the fractional proportion of deletion species that eliminate the ATG start codon. This increase in ATG-deleted alleles comes at the expense of deletions that produce species that fall into the other three categories, and further suggests that the PD-1 megaTAL-ExoX fusion may disproportionately erase undesired PD-1 gene edited alleles, such as in-frame single amino acid deletions to the N-terminal end of the signal sequence that may not impact PD-1 surface expression nor function. To assess the possibility that in-frame deletions may not functionally disable the PDCD1 locus, activated primary human T cell samples were electroporated with in vitro transcribed, capped and poly-adenylated mRNA encoding either or both a cyan fluorescent protein (CFP) to track transfection efficiency, and wild-type or mock-edited PD-1 alleles in each of the three possible reading frames. Flow cytometry was then performed to assess transfection efficiency and PD-1 protein expression, depicted in FIG. 16. While wild-type PD-1 mRNA (SEQ ID NO: 83) electroporation led to high level protein expression, artificial mRNAs encoding 1 or 2 base pair deletions (SEQ ID NOs: 84 and 85), mimicking frame 2 or frame 3 deletions species respectively, did not facilitate PD-1 protein expression as would be expected for mRNA encoding out-of-frame protein transcripts. In contrast, an artificial mRNA encoding a 3 base pair deletion at the PD-1 megaTAL target site (SEQ ID NO: 86) produced high levels of PD-1 surface expression, confirming in principle that some PD-1 gene editing allele species are phenotypically non-penetrant.

### Example 8

### Assessment of Editing at Off-Target Loci

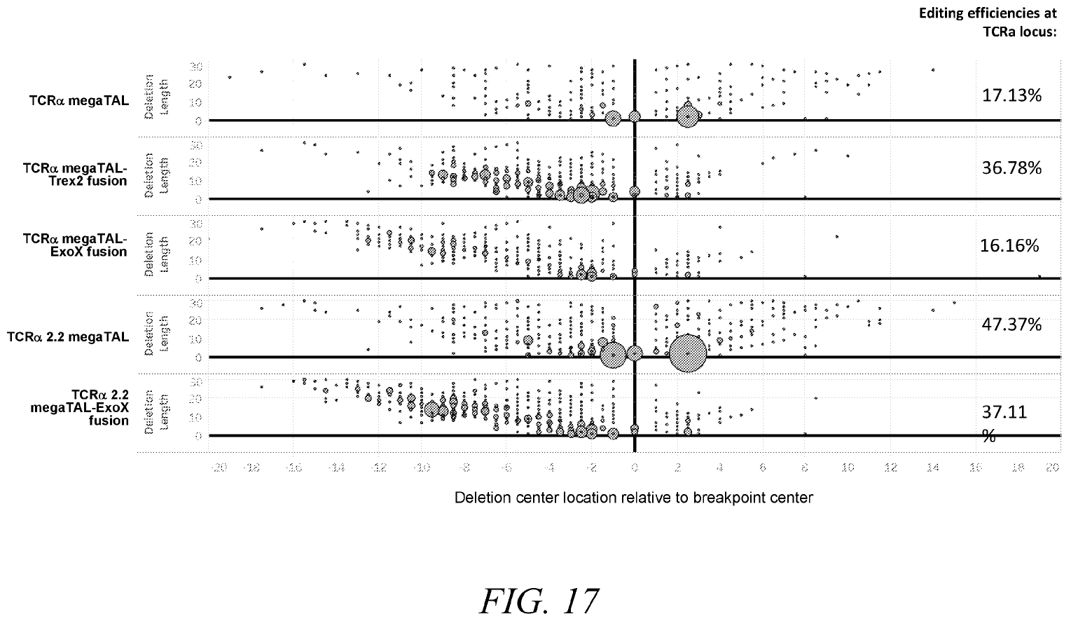

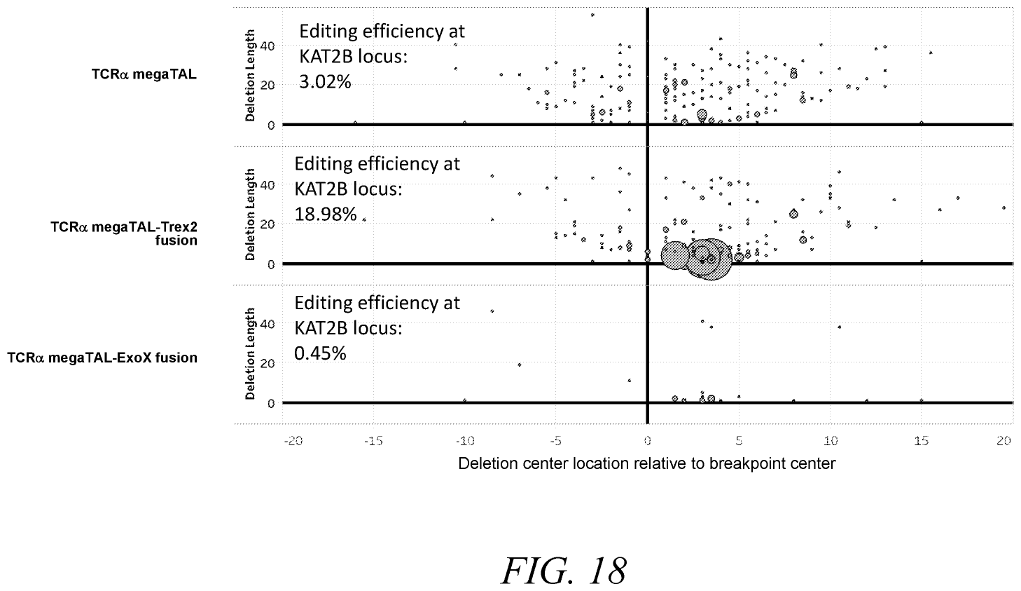

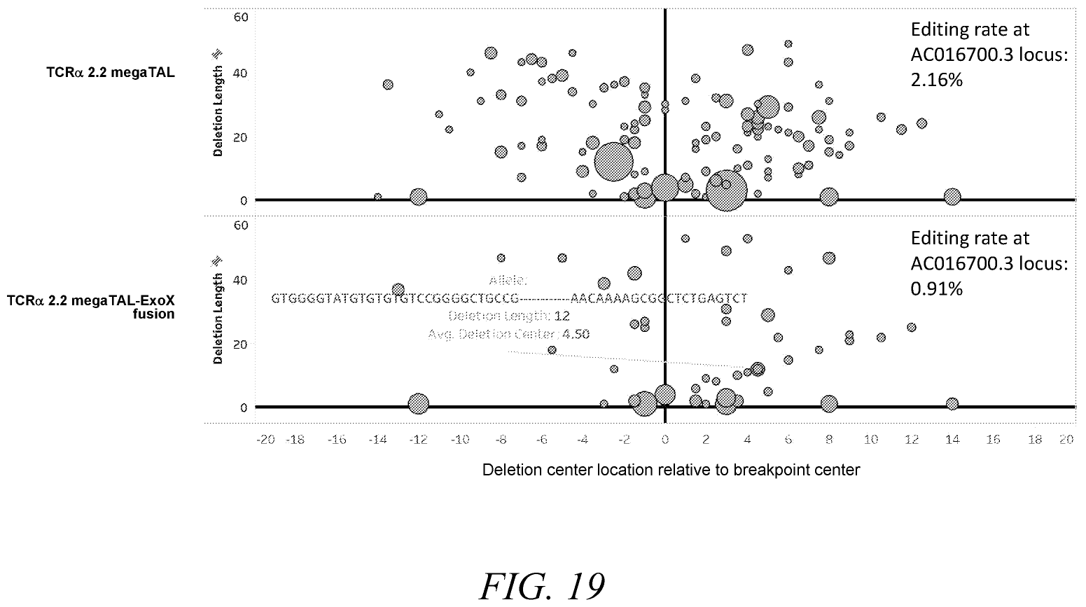

To further understand the qualitative aspects of the gene editing events at off-target sites, amplicon sequencing and deletion fingerprint analysis was performed at off-target sites specific to low and high editing TCRα megaTALs. Primary human T cells, activated and expanded from PBMCs, were electroporated with 1.5 ug mRNA encoding the low editing TCRα megaTAL, high editing TCRα megaTAL (TCRα 2.2), as well as direct fusions of each to Trex2 and direct fusions of each to ExoX. Each construct was evaluated at the on-target locus (FIG. 17; in duplicate; only one replicate shown), or its off-target locus (in duplicate, only one replicate shown). Following an outgrowth period of 7-10 days, cells were harvested for genomic DNA isolation. PCR was performed over an approximately 300 base pair region encompassing the KAT2B off-target site (susceptible to editing by the low editing TCRα megaTAL, FIG. 18) or the 2.2 off-target site AC016700.3 (susceptible to editing by the high editing TCRα megaTAL, FIG. 19). The PCR amplicons were then subjected to next-generation sequencing (NGS) and analyzed by tabulating deletion events according to their frequency (circle size) as well as both their length (y-axis) and their longitudinal location relative to the megaTAL caused breakpoint (x-axis). Insertions were excluded from this analysis. The TCRα 2.2 fusion to Trex2 failed to edit and so fingerprints are not shown. Those exonucleases that impacted the pattern of each TCRα megaTAL edit resulted in very low editing rates relative to the on-target editing efficiency of each TCRα megaTAL and showed changes in fingerprint. Because of the absence of substrate for the TALE array, there does not seem to be any biased affinity distribution with any of the megaTAL fusions tested.

1. A fusion polypeptide comprising, a DNA-binding domain and an I-OnuI homing endonuclease (HE) variant that binds and cleaves a selected double strand DNA (dsDNA) target site in a cell; a linker domain; and an exonuclease or biologically active fragment thereof.

2. The fusion polypeptide of claim 1, wherein the exonuclease is Trex2, ExoI, or ExoX, or biologically active fragment thereof.

3.-6. (canceled)

7. The fusion polypeptide of claim 1, wherein the DNA-binding domain binds a dsDNA target site upstream of the I-OnuI HE variant dsDNA target site.

8. The fusion polypeptide of claim 1, wherein the DNA-binding domain comprises a TALE DNA-binding domain.

9. The fusion polypeptide of claim 8, wherein the TALE DNA domain comprises about 9.5 TALE repeat units to about 15.5 TALE repeat units.

10. (canceled)

11. The fusion polypeptide of claim 1, wherein the DNA-binding domain comprises a zinc finger DNA-binding domain.

12. (canceled)

13. The fusion polypeptide of claim 1, wherein the linker domain is a peptide linker.

14.-30. (canceled)

31. The fusion polypeptide of claim 1, wherein the I-OnuI HE variant dsDNA target site is within a gene selected from the group consisting of: programmed cell death protein 1 (PD-1; PDCD1), lymphocyte activation gene 3 protein (LAG-3), T cell immunoglobulin domain and mucin domain protein 3 (TIM-3), cytotoxic T lymphocyte antigen-4 (CTLA-4), band T lymphocyte attenuator (BTLA), T cell immunoglobulin and immunoreceptor tyrosine-based inhibitory motif domain (TIGIT), V-domain Ig suppressor of T cell activation (VISTA), and killer cell immunoglobulin-like receptor (KIR), CCR5, TRAC (TCRa), TCRp, ILIORa, ILIORp, TGFBRI, TGFBR2, CBL-B, PCSK9, AHR, BTK, a-globin, p-globin, y-globin, BCLI IA, KLFI, SOX6, GATA1, LSDI, alpha folate receptor (FRa), avp6 integrin, B cell maturation antigen (BCMA), B7-H3 (CD276), B7-H6, carbonic anhydrase IX (CAIX), CD16, CD19, CD20, CD22, CD30, CD33, CD37, CD38, CD44, CD44v6, CD44v7/8, CD70, CD79a, CD79b, CD123, CD133, CD138, CDI71, carcinoembryonic antigen (CEA), C-type lectin-like molecule-I (CLL-1), CD2 subset 1 (CS-1), chondroitin sulfate proteoglycan 4 (CSPG4), cutaneous T cell lymphoma-associated antigen 1 (CTAGEI), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fe Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Ra, IL-I3Ra2, Kappa, cancer/testis antigen 2 (LAGE-IA), Lambda, Lewis-Y (LeY), L1 cell adhesion molecule (LI-CAM), melanoma antigen gene (MAGE)-AI, MAGE-A3, MAGE-A4, MAGE-A6, MAGEAIO, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (RORI), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), TEM5, TEM8, trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1) gene.

32.-33. (canceled)

34. The fusion polypeptide of claim 31, wherein the TCRa gene target site comprises the amino acid sequence set forth in SEQ ID NO: 1, the CBL-B gene target site comprises the amino acid sequence set forth in SEQ ID NO: 2, or the PD-1 gene target site comprises the amino acid sequence set forth in SEQ ID NO: 3.

35. (canceled)

36. The fusion polypeptide of claim 2, wherein the ExoX, or biologically active fragment thereof, comprises an amino acid an amino acid sequence having at least 85% identity to an amino acid sequence as set forth in SEQ ID NO: 109.

37. (canceled)

38. The fusion polypeptide of claim 36, comprising an amino acid sequence having at least 85% identity to an amino acid sequence as set forth in any one of SEQ ID NOs: 46, 64, 73, and 82.

39. (canceled)

40. The fusion polypeptide of claim 2, wherein the ExoI, or biologically active fragment thereof, comprises an amino acid an amino acid sequence having at least 85% identity to an amino acid sequence as set forth in SEQ ID NO: 112.

41. (canceled)

42. The fusion polypeptide of claim 40, comprising an amino acid sequence having at least 85% identity to an amino acid sequence as set forth SEQ ID NOs: 43.

43. (canceled)

44. A polynucleotide, a mRNA, or a vector encoding the fusion polypeptide of claim 1.

45.-53. (canceled)

54. A cell comprising the polypeptide of claim 1.

55.-69. (canceled)

70. A method of site-directed mutagenesis comprising:
a) selecting a double-stranded DNA (dsDNA) target site, and
b) introducing into the cell a fusion polypeptide of claim 1;
wherein the fusion peptide generates directionally biased deletions having a deletion center near the selected dsDNA target cut site in the cell.

71. (canceled)

72. The method of claim 70, wherein greater than 50% of the directionally biased deletions have a deletion center location on one side of the I-OnuI HE variant dsDNA target site center location.

73.-78. (canceled)

79. The method of claim 70, wherein the deletion center location is on the same side as the DNA-binding domain target site relative to the I-OnuI HE variant dsDNA target site center location.

80. The method of claim 70, wherein the deletion center location is 5′ to the I-OnuI HE variant dsDNA target site center location.

81. (canceled)

82. The method of claim 70, wherein at least 50% of deletions have a deletion center greater than 4 nucleotides away from the I-OnuI HE variant dsDNA target site center location, at least 10% of deletions have a deletion center greater than 8 nucleotides away from the I-OnuI HE variant dsDNA target site center location, or at least 35% of deletions are 12 bps in length or greater.

83.-110. (canceled)

111. The method of claim 70, wherein the deletion extends into the DNA-binding domain target site or the deletion center location is within the DNA-binding domain target site.

112.-119. (canceled)

120. A method of treating, preventing, or ameliorating at least one symptom of a disease, or condition associated therewith, comprising harvesting a population of cells from a subject; editing the population of cells according to the method of claim 70, and administering the edited population of cells to the subject.

121.-124. (canceled)
